(19) United States
(12) Patent Application Publication
(10) Pub. No.: US 2024/0024421 A1
(43) Pub. Date: Jan. 25, 2024

(54) RECOMBINANT MODIFIED FIBROBLAST GROWTH FACTORS AND THERAPEUTIC USES THEREOF

(71) Applicant: Trefoil Therapeutics, Inc., San Diego, CA (US)

(72) Inventor: David Eveleth, San Diego, CA (US)

(21) Appl. No.: 18/069,585

(22) Filed: Dec. 21, 2022

Related U.S. Application Data
(63) Continuation of application No. PCT/US21/39139, filed on Jun. 25, 2021.
(60) Provisional application No. 63/044,980, filed on Jun. 26, 2020.

(51) Int. Cl. A61K 38/18; A61K 47/12; A61K 47/22; A61K 47/26; A61P 27/02

(52) U.S. Cl. CPC A61K 38/1825; A61K 47/12; A61K 47/22; A61K 47/26; A61P 27/02

(57) ABSTRACT

Described herein are suitable formulations of modified fibroblast growth factor (FGF) and methods of making, for delivery in the eye, polypeptides, pharmaceutical compositions and medicaments that include such modified FGF polypeptides, and methods of using such modified FGF polypeptides to treat or prevent conditions that benefit from administration of FGFs.

### CROSS REFERENCE

This application is a continuation of International Patent Application No. PCT/US2021/039139, filed Jun. 25, 2021, which claims the benefit of U.S. Provisional Application No. 63/044,980, filed on Jun. 26, 2020; which is incorporated herein by reference in its entirety. REFERENCE TO SEQUENCE LISTING SUBMITTED ELECTRONICALLY

This application incorporates by reference a Sequence Listing submitted with this application as a text file entitled “Trefoil 45341-709.301_SL.xml” created on Dec. 1, 2022 and having a size of 312,378 bytes.

### FIELD OF THE INVENTION

Described herein are modified fibroblast growth factor (FGF) polypeptides, pharmaceutical compositions and medicaments that include such modified FGF polypeptides, and methods of preparing and using such modified FGF polypeptides to treat or prevent conditions that benefit from administration of FGFs.

### BACKGROUND OF THE INVENTION

FGFs are large polypeptides widely expressed in developing and adult tissues (Baird et al., Cancer Cells, 3:239-243, 1991) and play roles in multiple physiological functions (McKeehan et al., Prog. Nucleic Acid Res. Mol. Biol. 59:135-176, 1998; Burgess, W. H. et al., Annu Rev. Biochem. 58:575-606 (1989). The FGF family includes at least twenty-two members (Reuss et al., Cell Tissue Res. 313:139-157 (2003)).

### SUMMARY OF THE INVENTION

One embodiment provides a pharmaceutical formulation comprising:
a modified FGF-1 polypeptide,
citrate or histidine at a concentration of from about 1 mM to about 20 mM,
a surfactant at a concentration from about 0.01% to about 10% (w/v), and
a tonicity modifying agent at a concentration of from about 1% to about 10% (w/v) or from about 50 mM to about 200 mM,
   wherein the modified FGF-1 polypeptide comprises an amino acid sequence that is at least 90% identical to SEQ ID NO: 2 and comprises the following amino acid residues: Ser at position 16, Cys at position 66, and Val at position 117.

In some embodiments, the pharmaceutical formulation comprises the histidine at a concentration of about 1 mM or about 10 mM. In some embodiments, the concentration of the surfactant is about 0.1% (w/v). In some embodiments, the surfactant is a polysorbate. In some embodiments, the polysorbate is PS-20 or PS-80. In some embodiments, the polysorbate is PS-80. In some embodiments, the tonicity modifying agent is sorbitol and wherein the pharmaceutical formulation comprises sorbitol at a concentration of about 5% (w/v). In some embodiments, the pharmaceutical formulation has a pH of about 4.5 to about 6.5. In some embodiments, the pharmaceutical formulation has a pH of about 5.8. In some embodiments, the concentration of the modified FGF-1 polypeptide is from about 0.0005 μg/mL to about 200 μg/mL. In some embodiments, the concentration of the modified FGF-1 polypeptide is about 100 μg/mL. In some embodiments, the modified FGF-1 polypeptide is stable for at least 28 days when stored at room temperature, as measured by any one of: (i) lack of visible particulates by visual inspection and (ii) a peak area less than 5% for high molecular weight species in an SE-HPLC (size-exclusion-high performance liquid chromatography) assay. In some embodiments, the modified FGF-1 polypeptide is stable for at least 50 days when stored at room temperature. In some embodiments, the modified FGF-1 polypeptide stable for at least 59 days when the formulation is stored at room temperature. In some embodiments, formulation is suitable for topical application, application as eye drops, intraocular injection, or periocular injection. In some embodiments, the formulation is an injectable formulation for intraocular delivery. In some embodiments, the formulation is an injectable formulation for intravitreal delivery.

One embodiment provides a bulk drug substance formulation comprising a modified FGF-1 polypeptide; sodium chloride at a concentration from at least about 200 mM to about 1000 mM; ammonium sulfate at a concentration of about 50 mM to about 500 mM; di-sodium hydrogen phosphate at a concentration of about 1 mM to about 50 mM, wherein the modified FGF-1 polypeptide comprises an amino acid sequence that is at least 90% identical to SEQ ID NO:2 and comprises the following amino acid residues: Ser at position 16, Cys at position 66, and Val at position 117. In some embodiments, the concentration of the modified FGF-1 polypeptide is from at least about 0.1 g/mL to about 10 g/mL. In some embodiments, the concentration of the modified FGF-1 polypeptide is about 3 g/mL. In some embodiments, the bulk drug substance formulation comprises sodium chloride at a concentration of about 800 mM. In some embodiments, the bulk drug substance formulation comprises ammonium sulfate at a concentration of about 320 mM. In some embodiments, the bulk drug substance formulation comprises di-sodium hydrogen phosphate at a concentration of about 20 mM. In some embodiments, the bulk drug substance formulation has a pH of from about 7 to about 9. In some embodiments, the bulk drug substance formulation comprises has a pH of about 7.4. In some embodiments, the modified FGF-1 polypeptide in the bulk drug substance formulation is stable when stored at a temperature of −60° C.±10° C.

One embodiment provides a method of manufacture comprising purification of a refolded modified FGF-1 polypeptide isolated from inclusion bodies in a culture of bacterial cells transfected with a vector comprising a nucleic acid for encoding the modified FGF-1 polypeptide, wherein the purification comprises capturing of the refolded modified FGF-1 polypeptide highly cross-linked agarose base matrix coupled to dextran sulfate as ligand followed by polishing by hydrophobic interaction chromatography, using a chromatographic column packed with Butyl Sepharose resin. In some embodiments, the recovery of the modified FGF-1 polypeptide from the polishing step is about 10% to about 40% greater than recovery of the modified FGF-1 polypeptide after a polishing step by hydrophobic interaction chromatography, using a chromatographic column packed with Heparin resin, in a method of manufacture that is otherwise identical.

One embodiment provides a scalable method for producing a therapeutically effective modified FGF-1 polypeptide, the method comprising:
a. introducing a recombinant nucleic acid construct comprising a nucleic acid sequence encoding the modified FGF-1 polypeptide in an E. coli cell, wherein the construct is configured to target the translated modified FGF-1 polypeptide into the periplasmic space of the cell,
b. growing the cells in a synthetic growth media comprising a suitable antibiotic for about 20 hours; and
c. recovering from the cell, a therapeutically effective modified FGF-1 polypeptide,
wherein the yield of the modified FGF-1 recovered is at least 3 g/L in a scale of 1 L or more.

In some embodiments of the scalable method, the modified FGF-1 polypeptide comprises an amino acid sequence that is at least 90% identical to SEQ ID NO:2 and comprises the following amino acid residues: Ser at position 16, Cys at position 66, and Val at position 117. In some embodiments of the scalable method, the E. coli cell is selected from strain BL21, K12 HMS174, and W3110. In some embodiments of the scalable method, the recombinant nucleic acid construct is pMKet_TTHX1114 comprising a T7 or tac promoter. In some embodiments of the scalable method, the synthetic growth media comprises glycerol as carbon source, peptone and yeast. In some embodiments of the scalable method, the E. coli cells are BL21 cells and wherein the BL21 cells expressing pMKet_TTHX1114 are grown at 37° C. for about 20 hours in presence of kanamycin. In some embodiments of the scalable method, the recombinant nucleic acid construct comprises one or more modification for increasing yield of the modified FGF-1 polypeptide from the cell. In some embodiments of the scalable method, the one or more modifications comprise nucleic acid sequence codon optimization for increased expression of the modified FGF-1 polypeptide in the cell.

One embodiment provides a nucleic acid sequence for encoding a modified FGF1-1 polypeptide, the nucleic acid comprising the sequence of SEQ ID NO: 207. One embodiment provides a bacterial expression vector comprising the SEQ ID NO: 207, operably linked to a Tac promoter.

Provided herein in one embodiment is a recombinant modified FGF-1 polypeptide comprising the sequence set forth as SEQ ID NO: 1 with one or more mutations, wherein the polypeptide comprises an N-terminal methionine residue positioned upstream to the first residue of SEQ ID NO: 1. In some embodiments, the polypeptide further comprises an extension peptide positioned between the N-terminal methionine residue and the first residue of SEQ ID NO: 1. In some embodiments, the extension peptide comprises one or more amino acid residues of SEQ ID NO: 3. In some embodiments, the extension peptide comprises any one of the sequences set forth in SEQ ID NOS. 4-8. In some embodiments, the modified FGF-1 polypeptide is the mature form of the polypeptide. In some embodiments, the modified FGF-1 polypeptide comprises a sequence selected from SEQ ID NOS: 14-18.

Provided herein is a formulation, comprising: (a) a recombinant FGF-1 polypeptide comprising an amino acid sequence set forth in SEQ ID NO: 1, or having an amino acid sequence that is at least 90% identical to SEQ ID NO: 1, and comprising at least 1, 2, 3, 4 or 5 single amino acid mutations; and (b) L-methionine. In some embodiments, the formulation is an injectable formulation for intraocular delivery.

In some embodiments, the polypeptide further comprises an extension peptide positioned between the N-terminal methionine residue and the first residue of SEQ ID NO: 1. In some embodiments, the extension peptide comprises one or more amino acid residues of SEQ ID NO: 3.

In some embodiments, the extension peptide comprises any one of the sequences set forth in SEQ ID NOS. 4-8. In some embodiments, the modified FGF-1 polypeptide is the mature form of the polypeptide. In some embodiments, the modified FGF-1 polypeptide comprises a sequence selected from SEQ ID NOS: 14-18. In some embodiments, the modified FGF-1 polypeptide comprises a sequence selected from SEQ ID NOS: 24-28. In some embodiments, the modified FGF-1 polypeptide comprises a sequence selected from SEQ ID NOS: 93-117. In some embodiments, the polypeptide further comprises a methionine residue N-terminal to the extension peptide. In some embodiments, the modified FGF-1 polypeptide comprises a sequence selected from SEQ ID NOS: 118-141 and 207.

In some embodiments, the modified FGF-1 polypeptide is expressed in a form that comprises 136 amino acids. In some embodiments, the modified FGF-1 polypeptide comprises at least 141 amino acids in its mature form.

In some embodiments, the recombinant modified FGF-1 polypeptide comprising a mutation at position 67 of SEQ ID NO: 1.

In some embodiments, the modified FGF-1 polypeptide further comprises a truncation of one or more of the first five residues of SEQ ID NO: 1.

In some embodiments, the modified FGF-1 polypeptide comprises a sequence selected from SEQ ID NOS: 146-149.

In some embodiments, the polypeptide further comprises an extension peptide comprising one or more amino acid residues of SEQ ID NO: 3.

In some embodiments, the modified FGF-1 polypeptide comprises a sequence selected from SEQ ID NOS: 174-204.

In some embodiments, the polypeptide recombinant modified FGF-1 polypeptide comprising a sequence as set forth in SEQ ID NO: 2, or SEQ ID NO: 205.

In some embodiments, the modified FGF-1 polypeptide is the mature form of the polypeptide.

In some embodiments, the modified FGF-1 polypeptide comprises one or more mutations selected from the group consisting of: Cys16Ser, Ala66Cys, and Cys117Val.

In some embodiments, the modified FGF-1 polypeptide comprises one or more mutations of SEQ ID NO: 1, said mutation is selected from the group consisting of: Lys12Val, Cys16Ser, Ala66Cys, Cys117Val, and Pro134Val, and wherein the modified FGF-1 polypeptide further comprises at least one residue of the peptide ALTEK.

T In some embodiments, the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1, and at least one residue of the peptide ALTEK located between the N-terminal methionine and position 1 of SEQ ID NO: 1.

In some embodiments, the formulation comprises human serum albumin (HSA) and/or polysorbate 80.

In some embodiments, the formulation comprises L-methionine.

In some embodiments, the formulation comprises L-methionine and polysorbate 80.

In some embodiments, the formulation further comprising at least one of:
at least about 50 mM dibasic sodium phosphate dihydrate;
at least about 100 mM sodium chloride;
at least about 10 mM ammonium sulfate;
at least about 5 mM L-Methionine, and
at least about 0.01% polysorbate 80 (w/v).

In some embodiments, the formulation further comprising at least one of:
at least about 50 mM dibasic sodium phosphate dihydrate;
at least about 100 mM sodium chloride;
at least about 10 mM ammonium sulfate;
at least about 0.1 mM ethylenediaminetetraacetic acid (EDTA);
at least about 5 mM L-Methionine, and
at least about 0.01% polysorbate 80 (w/v).

In some embodiments, the formulation comprises the EDTA at a concentration of the EDTA is from at least about 0.01 mM to about 10 mM.

In some embodiments, the formulation comprises the ammonium sulfate, and wherein the concentration of the ammonium sulfate is from at least about 0.01 mM to about 100 mM.

In some embodiments, the formulation comprises the L-Methionine is from at least about 0.01 mM to about 100 mM.

In some embodiments, the recombinant FGF-1 is present at a concentration suitable for treating one or more diseases, disorders, or conditions selected from a list consisting of: Fuch's dystrophy, bullous keratopathy, herpetic keratopathy, congenital hereditary endothelial dystrophy 1, congenital hereditary endothelial dystrophy 2, posterior polymorphous corneal dystrophy, a dry eye syndrome, keratoconus, lattice corneal dystrophy, granular corneal dystrophy, macular corneal dystrophy, Schnyder crystalline corneal dystrophy, congenital stromal corneal dystrophy, fleck corneal dystrophy, corneal injury, ocular injury, chemical injury, vesicant injury, stromal injury and mustard gas keratopathy.

In some embodiments, provided herein is a pharmaceutical composition or formulation that facilitates administration of the modified FGF-1 polypeptides to an organism. Multiple techniques of administering a modified FGF-1 polypeptide exist in the art including, but not limited to: topical, ophthalmic, intraocular, periocular, intravenous, oral, aerosol, parenteral, and administration.

In some embodiments, the pharmaceutical composition is a liquid ophthalmic formulation. In some embodiments, the pharmaceutical formulation is administered topically, or by microneedle injection into the cornea, or intracamerally. Modes of local administration can include, for example, topical application, eye drops, intraocular injection or periocular injection. Periocular injection typically involves injection of the compound under the conjunctiva or into the Tennon's space (beneath the fibrous tissue overlying the eye). Intraocular injection typically involves injection of the modified FGF or pharmaceutical composition into the vitreous. In certain embodiments, the administration is non-invasive, such as by topical application or eye drops. In some embodiments, the administration is via a combination of topical and intracameral method.

In some embodiments, the formulation is administered intracamerally.

In some embodiments, the formulation is administered intravitreally.

In some embodiments, the formulation is stable for at least about 2 weeks to about 4 weeks, at a temperature of about −20° C.

Provided herein is a scalable method for producing a therapeutically effective modified FGF-1 polypeptide, the method comprising, (a) introducing a recombinant nucleic acid construct comprising a sequence encoding a modified FGF-1 polypeptide in a cell, wherein the recombinant nucleic acid construct comprises; wherein the modified FGF-1 polypeptide is produced by the cell; and (b) recovering from the cell, a therapeutically effective modified FGF-1 polypeptide. In some embodiments, the scalable method comprises. (a) introducing a recombinant nucleic acid construct in a suitable E. coli cell, wherein the recombinant nucleic acid construct comprises a sequence encoding the modified FGF-1 polypeptide for cytoplasmic expression, inserted in vector comprising a pBR322 derived ori-sequence, b. growing the cells in a synthetic growth media comprising a suitable antibiotic for about 20 hours; and c. recovering from the cell, a therapeutically effective modified FGF-1 polypeptide, wherein the yield of the modified FGF-1 recovered at step c is at least 2-fold higher than a method that does not comprise using a vector comprising a pBR322 derived ori-sequence, the synthetic growth media, or a combination thereof.

In some embodiments, the modified FGF-1 polypeptide comprises an amino acid sequence that is at least 90% identical to the amino acid sequence of SEQ ID NO: 1. In some embodiments, the modified FGF-1 polypeptide comprises one or more mutations of at positions 12, 16, 66, 117, and 134 of SEQ ID NO: 1. In some embodiments, the modified FGF-1 polypeptide comprises Ala66Cys mutation. In some embodiments, the modified FGF-1 polypeptide comprises a Cys16Ser mutation. In some embodiments, the modified FGF-1 polypeptide comprises a Cys 117Ser mutation. In some embodiments, the modified FGF-1 polypeptide comprises an N-terminal methionine residue positioned upstream to the first residue of SEQ ID NO: 1.

In some embodiments, the modified FGF-1 polypeptide further comprises an extension peptide positioned between the N-terminal methionine residue and the first residue of SEQ ID NO: 1. In some embodiments, the extension peptide comprises one or more amino acid residues of SEQ ID NO: 3.

In some embodiments, the extension peptide comprises any one of the sequences set forth in SEQ ID NOS. 4-8.

In some embodiments, the modified FGF-1 polypeptide is the mature form of the polypeptide.

In some embodiments, the modified FGF-1 polypeptide comprises a sequence selected from SEQ ID NOS: 14-18.

T In some embodiments, the modified FGF-1 polypeptide comprises a sequence selected from SEQ ID NOS: 24-28.

In some embodiments, the modified FGF-1 polypeptide comprises a sequence selected from SEQ ID NOS: 93-117.

In some embodiments, the modified FGF-1 polypeptide further comprises a methionine residue N-terminal to the extension peptide.

In some embodiments, the modified FGF-1 polypeptide comprises a sequence selected from SEQ ID NOS: 118-141 and 207.

In some embodiments, the modified FGF-1 polypeptide is expressed in a form that comprises 136 amino acids.

In some embodiments, the modified FGF-1 polypeptide comprises at least 141 amino acids in its mature form.

In some embodiments, the modified FGF-1 polypeptide further comprises a truncation of one or more of the first five residues of SEQ ID NO: 1.

In some embodiments, the modified FGF-1 polypeptide comprises a sequence selected from SEQ ID NOS: 146-149.

In some embodiments, the modified FGF-1 polypeptide further comprises an extension peptide comprising one or more amino acid residues of SEQ ID NO: 3.

In some embodiments, the modified FGF-1 polypeptide comprises a sequence selected from SEQ ID NOS: 174-204.

In some embodiments, the modified FGF-1 polypeptide comprises a sequence as set forth in SEQ ID NO: 2 SEQ ID NO: 205.

In some embodiments, the method is scalable to produce 1 g of modified FGF-1 polypeptide per batch.

In some embodiments, the method is scalable to produce 10 g of modified FGF-1 polypeptide per batch.

In some embodiments, the method is scalable to produce 100 g of modified FGF-1 polypeptide per batch.

In some embodiments, the method is scalable to produce 1 Kg of modified FGF-1 polypeptide per batch.

In some embodiments, the method is scalable to produce 10 Kg of modified FGF-1 polypeptide per batch.

In some embodiments, the method is scalable to produce 100 Kg of modified FGF-1 polypeptide per batch.

In some embodiments, the method is scalable to produce a batch preparation of at least 100 L cell stock.

In some embodiments, the cell is a yeast cell or a bacterium.

In some embodiments, the cell is a bacterium, wherein the bacterium is E. coli.

In some embodiments, the cell is an E. coli cell, strain BLA21A1.

In some embodiments, the cell is an E. coli cell, strain K12 HMS174, or W3110.

In some embodiments, the recombinant nucleic acid construct is in the form of a plasmid.

In some embodiments, the recombinant nucleic acid construct comprises one or more modification for increasing yield of the modified FGF-1 polypeptide from the cell.

The method of embodiment 69, wherein the one or more modifications comprise sequence optimization for increased expression of the modified FGF-1 polypeptide in the cell.

In some embodiments, the one or more modifications comprise modifications in the plasmid.

In some embodiments, the one or more modifications comprise selecting a suitable promoter for increasing yield of the modified FGF-1 polypeptide from the cell.

In some embodiments, the method further comprises growing cell in adequate nutrient media for maximizing cell proliferation.

In some embodiments, the adequate nutrient media comprises a carbon source.

In some embodiments, the carbon source is glucose or glycerol.

In some embodiments, the plasmid is pMKet, or a derivation or modification thereof.

In some embodiments, the method further comprises one or more modification processes for maximizing the yield of the modified FGF-1 polypeptide from the cell, wherein the one or more modification processes are selected from:
i. modification within the recombinant nucleic acid encoding the mutant FGF-1 polypeptide;
ii. modification within the recombinant nucleic acid comprising one or more regulatory elements related to the recombinant nucleic acid encoding the mutant FGF-1 polypeptide, selected from a promoter, an enhancer, a 5′-untranslated region, a 3′-untranslated region, a poly A tail, a transcript stabilizing element;
iii. modification of the plasmid comprising the recombinant nucleic acid;
iv. modification of the cell strain or selection of a cell strain for maximizing cell proliferation;
v. modification of the cell growth media; and
vi. modifications in the processes of recovering the modified FGF-1 polypeptide from the cell.

In some embodiments, introducing a recombinant nucleic acid comprises electroporating the recombinant nucleic acid in the cell.

In some embodiments, recovering the modified FGF-1 polypeptide from the cell comprises recovering the protein from periplasmic inclusion bodies of the cell.

In some embodiments, recovering comprises subjecting the inclusion bodies to solubilization in a denaturing buffer, and recovering FGF-1 polypeptide.

In some embodiments, the denaturing buffer comprises urea or guanidine.

The method of embodiment 34 or 77, wherein denaturing buffer comprises 6M guanidine at pH 7.4.

In some embodiments, the denaturing buffer further comprises 2 mM EDTA. In some embodiments, the method further comprises reducing the recovered FGF-1 polypeptide by adding DTT. In some embodiments, the method further comprises removing DTT by diafiltration.

In some embodiments, the recovered FGF-1 polypeptide is subjected to refolding in a refolding buffer.

In some embodiments, the refolding buffer comprises arginine.

In some embodiments, the refolding buffer comprises 1 M arginine.

In some embodiments, the refolding buffer comprises 5-50 mM Tris at pH 9-9.5.

In some embodiments, the refolding buffer comprises 5 mM Cysteine, or 2 mM Cystine or both.

In some embodiments, the FGF-1 is captured by hydrophobic interaction column (HIC) with heparin.

In some embodiments, the recovering a therapeutically effective recombinant mutant hFGF1 protein comprises purifying the protein. In some embodiments, purifying comprises one or more of: liquid chromatography, hydrophobic interaction chromatography, affinity chromatography, ultracentrifugation, transverse flow filtration, and diafiltration. In some embodiments, purifying comprises a step of purification through heparin column filtration. In some embodiments, purifying comprises recovering pure monomeric recombinant mutant hFGF1 protein. In some embodiments, purifying comprises recovering the pure monomeric recombinant mutant hFGF1 protein that is pathogen free, endotoxin free and substantially free of heparin.

Provided herein is a pharmaceutical composition comprising the modified FGF-1 polypeptide, produced by a method of any one of the embodiments 34-97, a lyophilized powder fraction thereof, or a liquid formulation thereof.

Provided herein is a plasmid vector comprising a recombinant nucleic acid sequence encoding a modified human FGF-1, the sequence operably linked to one or more regulatory sequences. In some embodiments, the recombinant nucleic acid sequence is designed for cytoplasmic expression. In some embodiments, the recombinant nucleic acid may encode a monocistronic sequence. In some embodiments, the recombinant nucleic acid may encode a polycistronic sequence. In some embodiments, the recombinant nucleic acid encodes a chaperone peptide sequence in addition to the FGF-1 polypeptide.

Provided herein is a method of treating a subject having a disease, a disorder, or a condition selected from a list consisting of: Fuch's dystrophy, bullous keratopathy, corneal ulcers, herpetic keratopathy, congenital hereditary endothelial dystrophy 1, congenital hereditary endothelial dystrophy 2, posterior polymorphous corneal dystrophy, a dry eye syndrome, keratoconus, lattice corneal dystrophy, granular corneal dystrophy, macular corneal dystrophy, Schnyder crystalline corneal dystrophy, congenital stromal corneal dystrophy, fleck corneal dystrophy, corneal injury, ocular injury, chemical injury, vesicant injury, stromal injury and mustard gas keratopathy, the method comprising administering to the subject in need thereof, a suitable dose of:
(i) the injectable formulation of any one of the embodiments 1-33, or
(ii) the pharmaceutical composition of embodiment 98.

Provided herein is a kit, comprising an injectable formulation of FGF-1. In some embodiments, the kit comprises a dropper bottle, wherein the dropper bottle is enabled to provide at least on dose of modified FGF-1 in the formulation or the pharmaceutical composition described herein. In some embodiments, the dropper bottle further comprises a sterile filter. In some embodiments, the container comprises the syringe. In some embodiments, the syringe comprises a material selected from the group consisting of tuberculin polypropylene and glass. In some embodiments, the kit comprises a unit dose container such as a blow-fill-seal dropper.

In some embodiments, the syringe is prefilled with an injectable formulation or a pharmaceutical composition described herein.

In some embodiments, the kit further comprises an electronic control unit. In some embodiments, the electronic control unit enables control of administration of a volume of an injectable formulation or a pharmaceutical composition described herein, wherein the volume is from at least about 10 μL to about 100 μL.

### INCORPORATION BY REFERENCE

All publications and patent applications mentioned in this specification are herein incorporated by reference in their entireties to the same extent as if each individual publication or patent application was specifically and individually indicated to be incorporated by reference.

### BRIEF DESCRIPTION OF DRAWINGS

FIG. 1 illustrates an exemplary generalized FGF-1 manufacturing process.

FIG. 2A illustrates an exemplary capture by butyl HIC chromatography and elution of the fraction shown by dotted lines SDS-PAGE yielded a single 17 kDa band corresponding to FGF-1 (not shown).

FIG. 2B illustrates an exemplary polishing step after butyl capture step, in heparin column, and representative SDS-PAGE of the eluents.

FIG. 3A and FIG. 3B illustrate data from experiments using different refolding buffers, captured in butyl and heparin HIC. FIG. 3C shows SDS-PAGE results of elution in butyl (left) followed by heparin HIC (right).

FIG. 4A and FIG. 4B show quantitative data indicating FGF1 recovery from representative experiments using urea or guanidine in refolding buffers, use of polysorbate 20 vs polysorbate 80, and comparing the resultant proteins run on SDS-PAGE. For each datapoint in the figures, the first column represents refolding at 4° C. and the second column represents refolding at room temperature.

FIG. 5 illustrates an exemplary generalized FGF-1 manufacturing process.

FIG. 6 illustrates a plasmid map for expression of a modified FGF-1 polypeptide in E. coli.

FIG. 7A, FIG. 7B, and FIG. 7C depict representative SEC-HPLC data showing clear peaks of the drug substance at day 0 (FIG. 7A), day 28 (FIG. 7B) and day 59 (FIG. 7C).

### DETAILED DESCRIPTION

Diseases of and injuries to the eyes can be severely debilitating, and occur in a wide variety of forms. One class of ocular disease is mustard gas keratopathy. Mustard gas is a vesicant poisonous gas that was first released by the German Army on a battlefield at Ypres in April 1915 during World War I. Exposure to mustard gas can lead to long-term complications, which develop over the years. The cornea becomes scarred and irregular, and cholesterol and calcium are deposited in its tissues, resulting in progressive impairment of vision. Slit-lamp examination reveals that the episcleral tissues display a characteristic underglaze. White porcelain appearance and unusual vascular anomalies are common. These appear as enlarged, distorted vessels, sometimes with an ampulliform outline accompanied by varicosities and sausage-like vessels. With the passage of time, dense opacification of the cornea results, being most evident in the central and lower sections, as the upper portion has been protected by the overhanging eyelid. Predominant histopathological features of MGK include, for example, irregular epithelial thickness, degenerative changes, thickened epithelial basement membrane, keratocytes loss, and destroyed Bowman layer. (Kanavi et al., Chronic and delayed mustard gas keratopathy: a histopathologic and immunohistochemical study, Eur. J Ophthalmol. 2010 September-October; 20(5):839-43). Typically, within one day of corneal vesicant exposure, the corneal epithelium (CE) sloughs from the basement membrane (BM), corneal edema develops in the denuded stroma and full-thickness keratocytosis is apparent within the wound margins. By five days, an epithelial cap is regenerated, and corneal edema begins to subside. One week after exposure, the CE is partially stratified, with rudimentary hemidesmosomal attachments. Despite this apparent improvement, corneas develop clinical signatures of chronic injury as soon as three weeks after exposure, including persistently elevated corneal edema, recurring corneal erosions and neovascularization. By eight weeks, the basement membrane zone undergoes severe degeneration. Further, MGK affected corneas appear to exhibit delayed wound healing process.

Provided herein are modified FGF-1 polypeptides, and liquid injectable formulations thereof, that include such modified peptides, and methods of using such modified FGF-1 polypeptides to treat various conditions, such as ocular disease, disorders and conditions (e.g., Fuch's dystrophy), vesicant agent induced corneal epithelial and endothelial injuries (e.g., Mustard Gas Keratopathy (MGK)), wound healing, cardiovascular diseases (e.g., ischemia), and neurological conditions (e.g., amylotrophic lateral sclerosis (ALS)). Also provided herein is a method of treating a chemical or vesicant induced injury by administering a modified fibroblast growth factors (FGF-1) polypeptides, or pharmaceutical composition or medicaments that include such modified peptides. In some embodiments, the method comprises treating mustard gas keratopathy (MGK), induced by a chemical injury, e.g., a chemical burn, by administering modified FGF-1 polypeptides described herein. In some embodiments, the method comprises treating mustard gas keratopathy (MGK), induced by a vesicant, e.g., nitrogen mustard (NM), by administering modified FGF-1 polypeptides described herein. In some embodiments, the method comprises treating a chemical or thermal injury caused by a chemical warfare agent, e.g., phosgene.

Also provided herein are methods of manufacturing modified FGF-1 polypeptides, and liquid injectable formulations thereof, that include such modified peptides, such that the modified FGF-1 polypeptides, and liquid injectable formulations thereof are suitable for using such modified FGF-1 polypeptides to treat various conditions, such as ocular disease, disorders and conditions (e.g., Fuch's dystrophy), vesicant agent induced corneal epithelial and endothelial injuries (e.g., Mustard Gas Keratopathy (MGK)), wound healing, cardiovascular diseases (e.g., ischemia), and neurological conditions (e.g., amylotrophic lateral sclerosis (ALS)). Also provided herein is a method of treating a chemical or vesicant induced injury by administering a modified fibroblast growth factors (FGF-1) polypeptides, or pharmaceutical composition or medicaments that include such modified peptides. In some embodiments, the method comprises treating mustard gas keratopathy (MGK), induced by a chemical injury, e.g., a chemical burn, by administering modified FGF-1 polypeptides described herein. In some embodiments, the method comprises treating mustard gas keratopathy (MGK), induced by a vesicant, e.g., nitrogen mustard (NM), by administering modified FGF-1 polypeptides described herein. In some embodiments, the method comprises treating a chemical or thermal injury caused by a chemical warfare agent, e.g., phosgene.

In some embodiments described herein, where the modified FGF-1 polypeptide is expressed with an N-terminal methionine (N-Met) residue, the polypeptide is subsequently purified without a step requiring proteolytic cleavage for removal of an N-terminal peptide. Accordingly, in some embodiments, the present disclosure provides a modified FGF-1 polypeptide that is prepared by a rapid purification method, without involving a proteolytic cleavage step for removal of an N-terminal peptide. This is particularly advantageous for production of the modified FGF-1 polypeptides per good manufacturing practice (GMP) guidelines. The advantages include the lack of a cleavage step, including eliminating the need for subsequent purification of the cleaved product and removal of the reagents used for cleavage. The further advantage of this is an increase in yield due to decreased handling and the alleviation of the need to test for residual cleavage reagents and contaminants introduced for the cleavage and subsequent separation of cleaved from uncleaved material.

The modified FGF-1 polypeptides described herein, can have increased stability (e.g. thermostability), reduced number of buried free thiols, and/or increased effective heparan sulfate proteoglycan (HSPG) affinity.

Several other advantages are associated with the use of the modified FGF-1 polypeptides in the methods described herein. For example, the modified FGF-1 polypeptides described herein can be administered without heparin in its pharmaceutical composition or formulation (e.g., an ophthalmic formulation), avoiding potential safety issues related to its biologic origin. In addition, avoidance of heparin allows the use of higher doses of the modified FGF-1 polypeptides without complications resulting from local heparin-induced adverse events or preexisting anti-heparin antibodies. Furthermore, in the absence of heparin, immediate binding of the modified FGF to tissue is maximized and systemic distribution is significantly reduced. The modified FGF-1 polypeptides described herein are also advantage of having enhanced local sequestration and reduced redistribution kinetics, thus increasing the elimination half-life and mean residence time (MRT) at the site of delivery, and allowing for a reduced dosing frequency. This can be the result of modified FGF-1 polypeptides described herein that have increased stability (e.g. thermostability), reduced number of buried free thiols, and/or increased effective heparan sulfate proteoglycan (HSPG) affinity.

The FGF-1 polypeptides of the present disclosure comprise, in various embodiments, modifications at the N-terminus of the polypeptide, such as an addition, a truncation, or a combination of additions and truncations. In some embodiments, the modification is the addition of a single N-terminal methionine residue. In some embodiments, the modification is the addition of an extension peptide. In some embodiments, the modification is a truncation of one or more of the first five residues of a FGF-1 polypeptide. In some embodiments, the FGF-1 polypeptides comprise a sequence as set forth in SEQ ID NO: 1, with one or more mutations, in addition to the N-terminal modification.

Several examples of the modified FGF-1 polypeptides disclosed herein comprise an N-terminal methionine (N-Met) residue in a mature form of the polypeptide. The retention of biological activity when amino acids are added to the N-terminus of a protein is unpredictable. Some proteins are tolerant of this and some are not, and the retention of biological activity and the potential for changes in stability are only determined empirically. The present disclosure identifies that the addition of N-terminal Met residues is tolerated with retention of biological activity and stability.

### Certain Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer softwares such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

Definition of standard chemistry terms may be found in reference works, including but not limited to, Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

The terms “treat,” “treating” or “treatment” include alleviating, abating or ameliorating a disease, disorder or condition symptoms, preventing additional symptoms, ameliorating or preventing the underlying metabolic causes of symptoms, inhibiting the disease, disorder, or condition, e.g., arresting the development of the disease, disorder or condition, relieving the disease, disorder or condition, causing regression of the disease, disorder or condition, relieving a condition caused by the disease, disorder or condition, or stopping the symptoms of the disease, disorder or condition. The terms “treat,” “treating” or “treatment”, include, but are not limited to, prophylactic and/or therapeutic treatments.

The term “acceptable” or “pharmaceutically acceptable”, with respect to a formulation, composition or ingredient, refers to having no persistent detrimental effect on the general health of the subject being treated or does not abrogate the biological activity or properties of the modified FGF described herein, and is relatively nontoxic.

The term “amelioration” of the symptoms of a particular disease, disorder or condition by administration of a particular modified FGF or pharmaceutical composition refers to any lessening of severity, delay in onset, slowing of progression, or shortening of duration, whether permanent or temporary, lasting or transient that can be attributed to or associated with administration of the modified FGF or pharmaceutical composition.

The term “combination” or “pharmaceutical combination” as used herein, means a product that results from the mixing or combining of more than one active ingredient and includes both fixed and non-fixed combinations of the active ingredients. The term “fixed combination” means that one active ingredient (e.g., a modified FGF) and a co-agent are both administered to a patient simultaneously in the form of a single entity or dosage. The term “non-fixed combination” means that one active ingredient (e.g., a modified FGF) and a co-agent are administered to a patient as separate entities either simultaneously, concurrently or sequentially with no specific intervening time limits, wherein such administration provides effective levels of the two agents in the body of the patient. The latter also applies to cocktail therapy, e.g., the administration of three or more active ingredients.

The term “pharmaceutical composition” or “pharmaceutical formulation,” as used interchangeably herein refers to a formulation comprising one or more modified FGF-1 polypeptides (e.g., a modified FGF-1 polypeptide of SEQ ID NO: 2) with one or more other chemical components, such as surfactants, buffering agents, tonicity modifying agents, carriers, stabilizers, diluents, dispersing agents, suspending agents, thickening agents, and/or other excipients, or any combination thereof. The pharmaceutical formulation facilitates administration of the modified FGF-1 polypeptides to an organism. Multiple techniques of administering a modified FGF-1 polypeptide exist in the art including, but not limited to: topical, ophthalmic, intraocular, periocular, intravenous, oral, aerosol, parenteral, and administration. In some embodiments, the pharmaceutical formulation comprises a modified FGF-1 polypeptide (e.g., a modified FGF-1 polypeptide comprising a sequence as set forth in SEQ ID NO: 2) as the active drug ingredient in association with inactive ingredients, e.g., a buffering agent, a surfactant, a tonicity modifying agent, or combinations thereof.

The term “carrier,” as used herein, refers to relatively nontoxic chemical compounds or agents that facilitate the incorporation of an agent of interest (e.g., a modified FGF) into cells or tissues.

The term “diluent” refers to chemical compounds that are used to dilute the agent of interest (e.g., a modified FGF) prior to delivery. Diluents can also be used to stabilize agents because they can provide a more stable environment. Salts dissolved in buffered solutions (which also can provide pH control or maintenance) are utilized as diluents in the art, including, but not limited to a phosphate buffered saline solution.

The terms “co-administration” or the like, are meant to encompass administration of the selected agents (e.g., a modified FGF or composition thereof and a co-agent) to a single patient, and are intended to include treatment regimens in which the agents are administered by the same or different route of administration or at the same or different time.

The terms “effective amount” or “therapeutically effective amount,” refer to a sufficient amount of a modified FGF-1 polypeptide, agent, combination or pharmaceutical composition described herein administered which will relieve to some extent one or more of the symptoms of the disease, disorder or condition being treated. The result can be reduction and/or alleviation of the signs, symptoms, or causes of a disease, or any other desired alteration of a biological system. For example, an “effective amount” for therapeutic uses is the amount of the modified FGF, agent, combination or pharmaceutical composition required to provide a desired pharmacologic effect, therapeutic improvement, or clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. It is understood that “an effect amount” can vary from subject to subject due to variation in metabolism of the modified FGF, combination, or pharmaceutical composition, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The term “prophylactically effective amount,” refers that amount of a modified FGF, compound, agent, combination or pharmaceutical composition described herein applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “subject” or “patient” as used herein, refers to an animal, which is the object of treatment, observation or experiment. By way of example only, a subject may be, but is not limited to, a mammal including, but not limited to, a human.

The terms “enhance” or “enhancing” means to increase or prolong either in potency or duration a desired effect. By way of example, “enhancing” the effect of therapeutic agents singly or in combination refers to the ability to increase or prolong, either in potency, duration and/or magnitude, the effect of the agents on the treatment of a disease, disorder or condition. When used in a patient, amounts effective for this use will depend on the severity and course of the disease, disorder or condition, previous therapy, the patient's health status and response to the drugs, and the judgment of the treating physician.

The term “modulate,” means to interact with a target (e.g., a FGF receptor) either directly or indirectly so as to alter the activity of the target, including, by way of example only, to enhance the activity of the target, to inhibit or antagonize the activity of the target, to limit the activity of the target, or to extend the activity of the target. In some embodiments, modified FGF-1 polypeptides and pharmaceutical compositions described herein can modulate the activity of one or more respective targets (e.g., one or more FGF receptors). In some embodiments, the modified FGF-1 polypeptides described herein modulate (e.g., increase) the activity of one or more FGF receptors on a cell (e.g., a corneal endothelial cell), resulting, e.g., in cell migration and/or cell proliferation.

As used herein, the term “target” or refers to a biological molecule (e.g., a target protein or protein complex), such as an FGF receptor, or a portion of a biological molecule capable of being bound by a selective binding agent (e.g., a modified FGF) or pharmaceutical composition described herein. As used herein, the term “non-target” refers to a biological molecule or a portion of a biological molecule that is not selectively bound by a selective binding agent or pharmaceutical composition described herein.

The term “target activity” or “cell response” refers to a biological activity capable of being modulated by a modified FGF or any cellular response that results from the binding of a modified FGF to a FGF receptor. Certain exemplary target activities and cell responses include, but are not limited to, binding affinity, signal transduction, gene expression, cell migration, cell proliferation, cell differentiation, and amelioration of one or more symptoms associated with an ocular disease, disorder or condition.

The terms “herpetic keratitis”, “herpes simplex keratitis”, “HSK”, “herpetic keratopathy”, “herpes cornea”, and “herpetic keratoconjunctivitis” refer to an ocular disease, disorder, or condition that is typically caused by herpes simplex virus (HSV).

### Expressed and Mature Forms of the Modified FGF-1 Polypeptides

FGFs stimulate a family seven FGF receptor isoforms, and each FGF stimulates a different pattern of receptors to achieve its specific effect. See, e.g., Ornitz et al. (1996) The Journal of biological chemistry, 1996, 271(25):15292-7; Zhang et al. (2006) The Journal of biological chemistry, 2006, 281(23):15694-700). In some embodiments, modified FGF-1 polypeptide is preferable because it binds to and stimulates all seven FGF receptor isoforms. See Ornitz et al. (1996) The Journal of biological chemistry, 1996, 271(25):15292-7.

Embodiments disclosed herein relate to a modified FGF-1 polypeptide or a pharmaceutical composition (e.g., an ophthalmic formulation) comprising a modified FGF-1 polypeptide. Embodiments disclosed herein also relate to a method of treating a chemical or a vesicant injury by administering a modified FGF-1 polypeptide or a pharmaceutical composition (e.g., an ophthalmic formulation) comprising a modified FGF-1 polypeptide. A modified FGF-polypeptide, as used herein, refers to a recombinant FGF that includes a substitution or mutation of one or more different amino acid residues and/or one or more deletions of one or more amino acid residues and/or one or more additions of one or more amino acid residues of SEQ ID NO: 1.

Provided herein, in a first embodiment, is a modified FGF-1 polypeptide comprising the sequence set forth as SEQ ID NO: 1, with one or more mutations, wherein the modified polypeptide further comprises a methionine residue upstream to the first residue of SEQ ID NO: 1. In some embodiments, the modified FGF-1 polypeptide comprising the N-terminal methionine (N-Met) residue is a mature form of the polypeptide. In some instances, the modified FGF-1 polypeptide, according to the first embodiment, comprises one or more mutations at positions 12, 16, 66, 117, and 134 of SEQ ID NO: 1. In some embodiments, the modified FGF-1 polypeptide is expressed in a host cell with a methionine residue upstream to the first residue of SEQ ID NO: 1. In some embodiments, the modified FGF-1 polypeptide is not subject to N-terminal processing for removal of the N-Met residue during maturation. Thus, in some embodiments, the mature form of a modified FGF-1 comprises an N-Met residue and one or more mutations at positions 12, 16, 66, 117, and 134 of SEQ ID NO: 1. An exemplary modified FGF-1 sequence, comprising an N-Met residue, is disclosed as SEQ ID NO: 2.

The present disclosure identifies that a modified FGF-1 as described herein, comprising an N-Met residue in its mature form, has similar biological activity as a version without the N-Met residue. N-terminal methionine removal, or excision, is a co-translational process that occurs as soon as a polypeptide emerges from the ribosome. The removal of the N-terminal methionine involves the substrate specificities of a cleavage enzyme, methionine aminopeptidase (metAP), which recognizes a methionine residue which is followed by an amino acid residue with a small side chain, such as alanine, glycine, proline, serine, threonine, or valine. Due to this substrate sequence specificity, the modified FGF-1 of the first embodiment, which comprises an N-Met residue followed by phenylalanine, see position 1 of SEQ ID NO: 1, is not processed by metAP. Thus, by expressing the modified FGF-1 with a methionine residue directly upstream of SEQ ID NO: 1, a mature modified FGF-1, comprising methionine as its N-terminal residue, can be obtained. In some embodiments, the modified FGF-1 according to the first embodiment is not expressed with an N-terminal peptide and therefore is not subject to proteolytic cleavage for removal of the same, during subsequent purification.

Provided herein, in a second embodiment, is a modified FGF-1 polypeptide comprising the sequence set forth as SEQ ID NO: 1, with one or more mutations, wherein the modified polypeptide further comprises a methionine residue upstream to the first residue of SEQ ID NO: 1, and one or more amino acids of the peptide set forth as SEQ ID NO: 3. A peptide comprising one or more residues of SEQ ID NO: 3 is herein referred to as an “extension peptide.” Thus, the modified FGF-1 according to the second embodiment comprises the sequence set forth as SEQ ID NO: 1, with one or more mutations, a methionine residue upstream to the first residue of SEQ ID NO: 1, and an extension peptide positioned between the methionine residue and the first residue of SEQ ID NO:1. In some embodiments, the modified FGF-1 polypeptide comprising the N-terminal methionine and an extension peptide, positioned between the methionine residue and the first residue of SEQ ID NO: 1, is a mature form of the polypeptide. In some embodiments, the modified FGF-1 polypeptide comprises one or more mutations at positions 12, 16, 66, 117, and 134 of SEQ ID NO: 1, which polypeptide is expressed in a host cell with a methionine residue upstream to the first residue of SEQ ID NO: 1, and further an extension peptide positioned between the methionine residue and the first residue of SEQ ID NO: 1. In some embodiments, the modified FGF-1 polypeptide according to the second embodiment is expressed with an extension peptide comprising five residues of SEQ ID NO: 3, positioned between the methionine residue and the first residue of SEQ ID NO: 1. In some embodiments, the modified FGF-1 polypeptide according to the second embodiment is expressed with four residues of SEQ ID NO: 3, positioned between the methionine residue and the first residue of SEQ ID NO: 1. In some embodiments, the modified FGF-1 polypeptide according to the second embodiment is expressed with three residues of SEQ ID NO: 3, positioned between the methionine residue and the first residue of SEQ ID NO: 1. In some embodiments, the modified FGF-1 polypeptide according to the second embodiment is expressed with two residues of SEQ ID NO: 3, positioned between the methionine residue and the first residue of SEQ ID NO: 1. In some embodiments, the modified FGF-1 polypeptide according to the second embodiment is expressed with one residue of SEQ ID NO: 3, positioned between the methionine residue and the first residue of SEQ ID NO: 1. Exemplary sequences of the extension peptide include SEQ ID NOS: 4-8.

In some instances, the modified FGF-1 polypeptide of the second embodiment, comprising an extension peptide and an N-terminal methionine residue, is not subject to N-terminal processing for removal of the methionine residue, whereas in some instances the methionine is excised by a cleavage enzyme. Typically, the cleavage enzyme is methionine aminopeptidase (metAP). Thus, in some examples, the mature form of the modified FGF-1 polypeptide according to the second embodiment comprises an N-Met residue followed by an extension peptide as described herein. Exemplary sequences of mature forms of modified FGF-1 polypeptides according to the second embodiment, comprising an N-terminal methionine, and one or more residues of the extension peptide, positioned between the methionine residue and the first residue of SEQ ID NO:1, are set forth as SEQ ID NOS: 9-13, wherein the sequences further comprise one or more mutations at amino acids corresponding to positions 12, 16, 66, 117, and 134 of SEQ ID NO: 1. Additional exemplar sequences of mature modified FGF-1 polypeptides comprising an N-terminal methionine, and an extension peptide are set forth as SEQ ID NOS: 14-18. In some other examples, the mature form of the modified FGF-1 polypeptide according to the second embodiment does not comprise an N-Met residue but includes only an extension peptide. Exemplary sequences of mature forms of modified FGF-1 polypeptides according to the second embodiment, comprising an extension peptide, positioned upstream to the first residue of SEQ ID NO:1 are set forth as SEQ ID NOS: 19-23, wherein the sequences further comprise one or more mutations at amino acids corresponding to positions 12, 16, 66, 117, and 134 of SEQ ID NO: 1. Additional exemplar sequences of mature modified FGF-1 polypeptides comprising one or more residues of the extension peptide are set forth as SEQ ID NOS: 24-28. In some embodiments, the methionine residue is cleaved by metAP when the extension peptide starts with an alanine (as in SEQ ID NO: 4) or with a threonine (as in SEQ ID NO: 5). In those instances, the mature FGF-1 polypeptide does not comprise an N-terminal methionine residue, e.g., SEQ ID NOS: 19, 21, 24, and 26.

Provided herein, in a third embodiment, is a modified FGF-1 polypeptide comprising the sequence set forth as SEQ ID NO: 1, with one or more mutations, wherein the modified polypeptide further comprises an extension peptide positioned upstream to the first residue of SEQ ID NO: 1. In some embodiments, the modified FGF-1 polypeptide comprising an extension peptide is a mature form of the polypeptide. In some embodiments, the modified FGF-1 polypeptide comprising one or more mutations at positions 12, 16, 66, 117, and 134 of SEQ ID NO: 1, which polypeptide is expressed in a host cell with one or more amino acid residues of the extension peptide positioned upstream to the first residue of SEQ ID NO: 1. Exemplary sequences of the modified FGF-1 polypeptides comprising an extension peptide, expressed without an N-terminal methionine residue, are set forth as SEQ ID NOS: 19-23, wherein the sequences further comprise one or more mutations at amino acids corresponding to positions 12, 16, 66, 117, and 134 of SEQ ID NO: 1. Additional exemplar sequences of mature modified FGF-1 polypeptides comprising one or more residues of the extension peptide, and expressed without an N-terminal methionine residue, are set forth as SEQ ID NOS: 24-28.

Provided herein, in a fourth embodiment, is a modified FGF-1 polypeptide comprising the sequence set forth as SEQ ID NO: 1, with one or more mutations, wherein the modified polypeptide further comprises a truncation of one or more of the first five residues of SEQ ID NO: 1. In some embodiments, the modified FGF-1 polypeptide comprising the truncation of one or more of the first five residues of SEQ ID NO: 1 is the mature form of the polypeptide. In some embodiments, the modified FGF-1 polypeptide comprises one or more mutations at positions 12, 16, 66, 117, and 134 of SEQ ID NO: 1, wherein one or more of the first five residues of SEQ ID NO: 1 is deleted. In some cases, the modified FGF-1 polypeptide comprising truncations is expressed with an N-terminal methionine residue. For instance, the modified FGF-1 polypeptide, according to the fourth embodiment, can have a sequence wherein the N-Met residue is followed by the second residue, asparagine, of SEQ ID NO: 1. In some cases, the modified FGF-1 polypeptide comprises an N-Met residue followed by the third residue, leucine, of SEQ ID NO: 1. In some cases, the modified FGF-1 polypeptide comprises an N-Met residue followed by the fourth residue, proline, of SEQ ID NO: 1. In some cases, the modified FGF-1 polypeptide comprises an N-Met residue followed by the fifth residue, proline, of SEQ ID NO:1. An extension peptide can be positioned in between the N-Met residue and the first, second, third, fourth, or fifth residue of SEQ ID NO: 1. Examples of a mature form of the modified FGF-1 polypeptide according to the fourth embodiment wherein an N-Met residue is followed by the second, third, fourth, or fifth residue of SEQ ID NO: 1 are shown in SEQ ID NOS: 37-40, wherein the sequences further comprise one or more mutations at amino acids corresponding to positions 12, 16, 66, 117, and 134 of SEQ ID NO: 1. Additional examples of modified FGF-1 polypeptides comprising truncations and an N-Met residue, are provided in SEQ ID NOS: 41-44.

The present disclosure also relates to modified FGF-1 polypeptides comprising one or more mutations of SEQ ID NO: 1, wherein the polypeptides are expressed with an N-Met residue followed by an extension peptide, and the extension peptide is followed by truncation of one or more of the first five residues of SEQ ID NO: 1. In some embodiments, the modified FGF-1 polypeptide comprises one or more mutations at positions 12, 16, 66, 117, and 134 of SEQ ID NO: 1, wherein the polypeptide is expressed with an N-Met residue followed by an extension peptide, and the extension peptide is followed by truncation of one or more of the first five residues of SEQ ID NO: 1. Examples of such sequences expressed with an N-Met residue followed by an extension peptide, which extension peptide is followed by truncation of one or more of the first five residues of SEQ ID NO: 1 are disclosed as SEQ ID NOS: 45-68, wherein the sequences further comprise one or more mutations at amino acids corresponding to positions 12, 16, 66, 117, and 134 of SEQ ID NO: 1. In some examples, the N-terminal methionine is cleaved off by N-terminal processing and accordingly the mature form of the modified FGF-1 polypeptide comprises only one or more residues of the leader fragment followed by truncation of one or more of the first five residues of SEQ ID NO: 1, as exemplified in SEQ ID NOS: 69-92, wherein the exemplary sequences further comprise one or more mutations at amino acids corresponding to positions 12, 16, 66, 117, and 134 of SEQ ID NO: 1. Additional examples of sequences without N-Met residue but including an extension peptide and truncations of N-terminal residues, are provided in SEQ ID NO: 93-117.

In some examples, the N-Met residue is retained in the mature modified FGF-1 polypeptide sequence, and accordingly the mature forms comprise sequences as exemplified in SEQ ID NO: 45-68, further comprising one or more mutations at amino acids corresponding to positions 12, 16, 66, 117, and 134 of SEQ ID NO: 1. Additional examples of sequences comprising an N-Met residue, an extension peptide and truncations of N-terminal residues, are provided in SEQ ID NO: 118-141 and 207.

The truncated versions of the modified FGF-1 polypeptides comprising one or more mutations at positions 12, 16, 66, 117, and 134 of SEQ ID NO: 1, are, in a fifth embodiment, expressed without an N-terminal methionine residue, and further without an extension peptide. In some examples, mature modified FGF-1 polypeptides according to the fifth embodiment comprise a sequence as set forth in SEQ ID NOS: 29-32, wherein the sequences further comprise one or more mutations at amino acids corresponding to positions 12, 16, 66, 117, and 134 of SEQ ID NO: 1. In some examples, the modified FGF-1 polypeptides according to the fifth embodiment comprise a sequence selected from the group consisting of SEQ ID NOS: 33-36.

In instances where the modified FGF-1 polypeptide, or its truncated version, comprising one or more mutations at positions 12, 16, 66, 117, and 134 of SEQ ID NO: 1, is expressed with an N-terminal methionine followed by an extension peptide, the methionine residue is either retained or cleaved off of the N-terminus during maturation of the polypeptide after expression. In some examples, where the modified FGF-1 polypeptide is expressed with an alanine next to the N-Met residue, e.g., SEQ ID NO: 14, the methionine is cleaved, to yield a mature FGF-1 polypeptide that does not comprise an N-Met residue, e.g., SEQ ID NO: 19. In some examples, where the modified FGF-1 polypeptide is expressed with a threonine next to the N-Met residue, e.g., SEQ ID NO: 16, the methionine is cleaved, to yield a mature FGF-1 polypeptide that does not comprise an N-Met residue, e.g., SEQ ID NO: 20. In some examples, where the modified FGF-1 polypeptide is expressed with a glutamic acid next to the N-Met residue, e.g., SEQ ID NO: 17, the methionine is not cleaved, to yield a mature FGF-1 that comprise an N-terminal methionine and has the same sequence as the expressed form.

Provided herein, in a sixth embodiment, is a modified FGF-1 polypeptide comprising the sequence set forth as SEQ ID NO: 1, comprising a mutation at position 67. In some embodiments, the modified FGF-1 polypeptide comprises a mutation at position 67 of SEQ ID NO: 1, one or more further mutations at positions 12, 16, 66, 117, and 134, and is expressed with an N-Met residue. The internal methionine at position 67 can be replaced, for example, with an alanine residue. In absence of the internal methionine at position 67, the N-terminal methionine of the modified FGF-1 polypeptide can be cleaved, post-expression; using cyanogen bromide (CNBr), an agent that specifically cleaves the amide bond after methionine residues. In some cases, the modified FGF-1 polypeptides according to the sixth embodiment are expressed with an extension peptide. In some other cases, modified FGF-1 polypeptides according to the sixth embodiment are expressed in a form comprising truncations of one or more of the first five residues of SEQ ID NO: 1, as exemplified in SEQ ID NOS: 142-149, wherein the sequences further comprise one or more mutations at amino acids corresponding to positions 12, 16, 66, 117, and 134 of SEQ ID NO: 1. In yet other examples, the modified FGF-1 polypeptides according to the sixth embodiment are expressed in a form comprising an extension peptide and truncations of one or more of the first five residues of SEQ ID NO: 1, as exemplified in SEQ ID NOS: 151-175. Additional examples of the modified FGF-1 polypeptides according to the sixth embodiment, in their mature forms, are set forth in SEQ ID NOS: 174-204. Among the modified FGF-1 polypeptides expressed in a form that comprises an internal methionine mutation, in cases where the polypeptide is expressed with an N-terminal methionine followed by an alanine or a threonine residue from the extension peptide, e.g., SEQ ID NO: 175 and SEQ ID NO: 177, respectively, the N-terminal methionine can be cleaved off during maturation of the polypeptide either by metAP or using CNBr.

Provided herein, in a seventh embodiment, is a modified FGF-1 polypeptide comprising the sequence set forth as SEQ ID NO: 205, for use in a method as described herein. Provided herein, in an eighth embodiment, is a modified FGF-1 polypeptide comprising the sequence set forth as SEQ ID NO: 206, for use in a method as described herein

The present disclosure further relates to modified FGF-1 polypeptides comprising any combination of deletion, insertion, and substitution of SEQ ID NO: 1, provided that said modified polypeptide comprises one or more mutations of SEQ ID NO: 1. Amino acid substitutions may be introduced into a modified FGF-1 polypeptide and the products screened for a desired activity, e.g., retained/improved effectivity in treating ocular disorders, increased potency in amelioration of Fuch's dystrophy, improved treatment of mustard gas keratopathy. Amino acid substitutions may also be introduced into a modified FGF-1 polypeptide and the products screened for a desired physicochemical property, e.g., less prone to aggregation, improved solubility, prolonged half-life, ease of formulating as an ophthalmic pharmaceutical, enhanced stability, improved shelf-life. Both conservative and non-conservative amino acid substitutions are contemplated.

The modified FGF-1 polypeptide, as in any of the above embodiments, is expressed in a form that comprises at least 136 amino acids. In some embodiments, the modified FGF-1 polypeptide is expressed in a form that comprises 137 amino acids. In some embodiments, the modified FGF-1 polypeptide is expressed in a form that comprises 138 amino acids. In some embodiments, the modified FGF-1 polypeptide is expressed in a form that comprises 139 amino acids. In some embodiments, the modified FGF-1 polypeptide is expressed in a form that comprises 140 amino acids. In some embodiments, the modified FGF-1 polypeptide is expressed in a form that comprises 141 amino acids. In some embodiments, the modified FGF-1 polypeptide is expressed in a form that comprises 142 amino acids. In some embodiments, the modified FGF-1 polypeptide is expressed in a form that comprises 143 amino acids. In some embodiments, the modified FGF-1 polypeptide is expressed in a form that comprises 144 amino acids. In some embodiments, the modified FGF-1 polypeptide is expressed in a form that comprises 145 amino acids. In some embodiments, the modified FGF-1 polypeptide is expressed in a form that comprises 146 amino acids.

The modified FGF-1 polypeptide, as in any of the above embodiments, comprises at least 136 amino acids in the mature form. In some examples, the modified FGF-1 polypeptide comprises 137 amino acids in the mature form. In some examples, the modified FGF-1 polypeptide comprises 138 amino acids in the mature form. In some examples, the modified FGF-1 polypeptide comprises 139 amino acids in the mature form. In some examples, the modified FGF-1 polypeptide comprises 140 amino acids in the mature form. In some examples, the modified FGF-1 polypeptide comprises 141 amino acids in the mature form. In some examples, the modified FGF-1 polypeptide comprises 142 amino acids in the mature form. In some examples, the modified FGF-1 polypeptide comprises 143 amino acids in the mature form. In some examples, the modified FGF-1 polypeptide comprises 144 amino acids in the mature form. In some examples, the modified FGF-1 polypeptide comprises 145 amino acids in the mature form. In some examples, the modified FGF-1 polypeptide comprises 146 amino acids in the mature form.

In some embodiments, the sequence of the modified FGF-1 polypeptide comprises 50%, 55%0, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, provided that said polypeptide comprises an N-Met residue in the mature form of the polypeptide. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 9-13, provided that said polypeptide comprises the N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 14-18, provided that said polypeptide comprises the N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 19-23, provided that said polypeptide does not comprise the N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 24-28, provided that said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 19-23, provided that said polypeptide does not comprise an N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 37-40, provided that said polypeptide comprises an N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%7, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 41-44, provided that said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 45-68, provided that said polypeptide comprises one or more mutations at amino acid positions corresponding to positions 12, 16, 66, 117, and 134 of SEQ ID NO: 1, and said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 69-92, comprises one or more mutations at amino acid positions corresponding to positions 12, 16, 66, 117, and 134 of SEQ ID NO: 1, and said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 93-117, provided that said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 118-141 and 207, provided that said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 29-32, provided that said polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 33-36.

In some embodiments, the sequence of the modified FGF-1 polypeptide comprises 50%, 55%0, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 142-204.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 12 with, for example, the mutation Lys12Val, and wherein said modified FGF-1 polypeptide comprises an N-terminal methionine in its mature form. In some embodiments, the modified FGF-1 polypeptide comprises a sequence with a mutation at position 12 of SEQ ID NO: 1, for example the mutation Lys12Val, and with truncation of one or more of the first five residue of SEQ ID NO: 1, wherein said modified FGF-1 polypeptide comprises an N-Met residue in its mature form. In some embodiments, the modified FGF-1 polypeptide comprises a sequence with a mutation at position 12 of SEQ ID NO: 1, for example the mutation Lys12Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 12 of SEQ ID NO: 1, for example the mutation Lys12Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 16 with, for example, the mutation Cys16Ser, and wherein said modified FGF-1 polypeptide comprises an N-met residue in its mature form. In some embodiments, the modified FGF-1 polypeptide comprises a sequence with a mutation at position 16 of SEQ ID NO: 1, for example the mutation Cys16Ser, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 16 of SEQ ID NO: 16, for example the mutation Cys16Ser, with an extension peptide, and with truncation of one or more of the first five residue of SEQ ID NO: 1, wherein said modified FGF-1 polypeptide comprises an N-met residue. In some embodiments, the modified FGF-1 polypeptide comprises a sequence with mutations at position 16 of SEQ ID NO: 1, for example the mutation Cys16Ser, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 66 with, for example, the mutation Ala66Cys, and wherein said modified FGF-1 polypeptide comprises an N-terminal methionine in its mature form. In some embodiments, the modified FGF-1 polypeptide comprises a sequence with a mutation at position 66 of SEQ ID NO: 1, for example the mutation Ala66Cys, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 66 of SEQ ID NO: 1, for example the mutation Ala66Cys, with an extension peptide, and with truncation of one or more of the first five residue of SEQ ID NO: 1, wherein said modified FGF-1 polypeptide is expressed with an N-Met residue. In some embodiments, the modified FGF-1 polypeptide comprises a sequence with mutations at position 66 of SEQ ID NO: 1, for example the mutation Ala66Cys, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 117 with, for example, the mutation Cys117Val, and wherein said modified FGF-1 polypeptide comprises an N-met residue in its mature form. In some embodiments, the modified FGF-1 polypeptide comprises a sequence with a mutation at position 117 of SEQ ID NO: 1, for example the mutation Cys117Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 117 of SEQ ID NO: 1, for example the mutation Cys 117Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 117 of SEQ ID NO: 1, for example the mutation Cys117Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 134 with, for example, the mutation Pro134Val, and wherein said modified FGF-1 polypeptide comprises an N-terminal methionine in its mature form. In some embodiments, the modified FGF-1 polypeptide comprises a sequence with a mutation at position 134 of SEQ ID NO: 1, for example the mutation Pro134Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 134 of SEQ ID NO: 1, for example the mutation Pro134Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 134 of SEQ ID NO: 1, for example the mutation Pro134Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at positions 16,66, and 117 of SEQ ID NO: 1, with, for example, the mutation Cys16Ser, Ala66Cys, and Cys117Val, and wherein said modified FGF-1 polypeptide comprises an N-met residue in its mature form. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, with an extension peptide, and with truncation of one or more of the first five residue of SEQ ID NO: 1, wherein said modified FGF-1 polypeptide comprises an N-met residue. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to a sequence selected from SEQ ID NOs: 2 and 9-204. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 205 or 206.

In some embodiments, the modified FGF-1 polypeptide is thermostable. As used herein, a thermostable FGF (e.g., a thermostable FGF-1) refers to an FGF having a modified amino acid sequence relative to SEQ ID NO: 1 that is also more stable than the polypeptide of SEQ ID NO: 1 under the same conditions. Examples of mutations capable of conferring thermostability to FGF (e.g., FGF-1) and methods for assessing thermostability are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. (2012) 7(11):e48210. In some embodiments, positions 12 and/or 134 are mutated in FGF-1 to generate a modified FGF-1 that is thermostable. An FGF-1 formulation may be considered “stable” for a duration of time at a certain temperature, which is understood as the formulation in which the FGF-1 is present in its original purity and form for the designated period of time at the designated temperature. In some embodiments, the FGF-1 may be considered as remaining in its original purity and form, if there is less than 5%, less than 2% or preferably less than 1% degradation or change in its monomeric form. Such a change may be detectable by any of the analytic procedures discussed herein, for example, chromatographic procedures, ELISA, SDS-PAGE and western blot.

In some embodiments, the modified FGF-1 polypeptide includes one or more modifications that reduce the number of reactive thiols (e.g., free cysteines). Examples such modifications in FGF-1 are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. (2012) 7(11):e48210. In some embodiments, positions 83 and/or 117 are mutated in SEQ ID NO: 1 to generate a modified FGF-1 that reduces the number of reactive thiols.

In some embodiments, the modified FGF includes one or more modifications that enable formation of an internal disulfide linkage. In some embodiments, position 66 is mutated in SEQ ID NO: 1 to generate a modified FGF-1 that comprises an internal disulfide linkage.

In some embodiments, the modified FGF-1 polypeptides described herein can be administered without exogenous heparin in the formulation for stability, they can be formulated and applied without heparin and thus are more able to bind to the tissue heparans. Such modified FGF-1 polypeptides have a high affinity for tissue heparans that are exposed in a surgical, traumatic or dystrophic conditions and disease-states and so bind to diseased tissue on application. In addition, the modified FGF-1 polypeptides being more thermally stable are suitable for formulation and storage at room temperature. The stability of the modified FGF-1 polypeptides also makes them suitable for administration in both solution (e.g., immediate release) and sustained-release formulations.

In some embodiments, the modified FGF-1 polypeptide is SEQ ID NO: 1 that has been modified at one or more of positions 12, 16, 66, 117, and 134. In some embodiments, the modified FGF is SEQ ID NO: 1 that has been modified at positions 16, 66, and 117. The amino acid positions can be substituted with, e.g., Ser, Cys, Val, or other amino acids to create disulfide linkages between modified amino acids and wild-type amino acids. In some embodiments, the modified FGF comprises the amino acid sequence of SEQ ID NO: 2, also referred to as N-Met THX1114. In some embodiments, the modified FGF-1 polypeptide comprises one or more mutations selected from the group consisting of: Lys12Val, Pro134Val, Ala66Cys, Cys117Val, and Pro134Val. In some embodiments, the modified FGF-1 polypeptide comprises the sequence of SEQ ID NO: 2.

In some embodiments, the modified FGF-1 polypeptides or compositions described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug.

The modified FGF-1 polypeptides described herein may be labeled isotopically (e.g., with a radioisotope) or by other means, including, but not limited to, the use of chromophores or fluorescent moieties, bioluminescent labels, photoactivatable or chemiluminescent labels.

The present discloser further relates to modified FGF polypeptides comprising N-terminal modification(s), wherein the modified FGF polypeptide can be any member of the FGF family, including FGF-1 (SEQ ID NO: 1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23, and FGF-24.

In some embodiments, the synthesis of modified FGF-1 polypeptides as described herein is accomplished using means described in the art, using the methods described herein, or by a combination thereof.

In some embodiments, the sequence of the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at one or more positions 16, 66, and 117 with, for example, the mutations Cys16Ser, Ala66Cys, and Cys 117Val. In some embodiments, the modified FGF comprises the wild-type human FGF-1 sequence with a mutation at positions 16, 66 and 117, for example the mutations Cys16Ser, Ala66Cys, and Cys 117Val.

### Recombinant Techniques for Preparation of Modified FGF-1 Polypeptides

A variety of host-expression vector systems may be utilized to produce the modified FGF-1 polypeptides provided herein. Such host-expression systems represent vehicles by which the modified FGF-1 polypeptides may be produced and subsequently purified, but also represent cells that may, when transformed or transfected with the appropriate nucleotide coding sequences, exhibit the modified gene product in situ. Examples of host-expression systems include but are not limited to, bacteria, insect, plant, mammalian, including human host systems, such as, but not limited to, insect cell systems infected with recombinant virus expression vectors (e.g., baculovirus) containing nucleotide sequences coding for the modified FGF-1 polypeptides; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences for the modified FGF-1 polypeptides; or mammalian cell systems, including human cell systems, e.g., HT1080, COS, CHO, BHK, 293, 3T3, harboring recombinant expression constructs containing promoters derived from the genome of mammalian cells, e.g., metallothionein promoter, or from mammalian viruses, e.g., the adenovirus late promoter; the vaccinia virus 7.5K promoter, or from yeast-derived plasmids e.g., pSH19 and pSH15, or from bacteriophages such as lambda phase and derivatives thereof. Examples of bacterial expression systems include but are not limited to Escherichia coli-derived plasmids (e.g., pMKet, pBR322, pBR325, pUC12, pUC13, and pET-3); Bacillus subtilis-derived plasmids (e.g., PUB110, pTP5, and pC194). In some embodiments the bacterial expression system comprises a pMKet vector. In some embodiments, a method comprising use of the pMKet bacterial expression vector to express modified FGF-1 improves yield of the modified FGF-1 by about 5 fold to about 60-fold, compared to a method comprising subcloning a sequence encoding the modified FGF-1 into a pET vector. In some embodiments, the method comprising the subcloning of modified FGF1 in a pET vector results in a yield of about 0.5 g-about 0.7 g/100 L following a fermentation run. In some embodiments, the method comprising the subcloning of modified FGF1 in a pMKet vector results in a yield of about 20 g-about 40 g/100 L following a fermentation run, for example 37 g/100 L. In some embodiments, the method comprising the subcloning of modified FGF1 in a pMKet and purifying the protein from the vector using the techniques described herein results in a yield of about 82 g/50 L.

In some embodiments, a host cell strain is chosen such that it modulates the expression of the inserted sequences, or modifies and processes the gene product in the specific fashion desired. Such modifications and processing of protein products may be important for the function of the protein. Different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and W138.

In some embodiments, bacterial cells are used for expressing the recombinant FGF protein. In some embodiments the bacterial cell in an E. coli cell. In some embodiments, the E. coli strain is selected from BL21, BLA21A1, K12 HMS174, and W3110. For long-term, high-yield production of recombinant peptides, stable expression is desired. For example, cell lines that stably express the recombinant modified FGF-1 polypeptides may be engineered. In some embodiments, rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. In some embodiments, the recombinant nucleic acid comprising a sequence encoding the FGF-1 polypeptide optimized for maximizing codon usage by the strain or cell in which it is expressed. In some embodiments, the recombinant nucleic acid comprising a sequence encoding the FGF-1 polypeptide is operably linked to a promoter, 3′ UTR regulatory sequence, for example a poly A sequence or a sequence that stabilizes the transcript, and helps in translation. In some embodiments the plasmid vector comprises a selection marker, such as an antibiotic resistance gene, such as kanamycin.

In some embodiments the promoter for bacterial expression is a T7 promoter.

In some embodiments the promoter for bacterial expression is a Tac promoter.

In some embodiments, the plasmid comprises a pBR322 on sequence.

In some embodiments, the bacterial expression vector is modified from a commercially available vector backbone, such as pBR322, pBR325, pUC12, pUC13, and pET-T3, or pET-T7 vectors.

In some embodiments, a periplasmic expression of the protein is intended. The recombinant protein may accumulate in inclusion bodies. In some embodiments, cytoplasmic expression of the protein is intended. In some embodiments, the cytoplasmic expression of the protein is intended, wherein the protein is an insoluble protein. In some embodiments, the recombinant polypeptide may be desired for extracellular release. In some embodiments, the recombinant nucleic acid encoding the polypeptide may comprise a suitable leader sequence, such as an ompA leader sequence. In some embodiments, the recombinant nucleic acid encoding the modified FGF-1 polypeptide does not comprise a leader sequence, such as an ompA leader sequence. In some embodiments, during manufacture, the modified FGF-1 polypeptide, in certain step, is directed to the periplasmic space. The periplasmic space comprises inclusion bodies, where the polypeptide is likely to accumulate. Inclusion bodies may then be harvested after cell fractionation to recover the polypeptide. In some embodiments, the recombinant nucleic acid does not contain a leader sequence. In some embodiments the modified FGF-1 is directed for cytoplasmic expression in the cell.

In some embodiments, the recombinant nucleic acid construct comprises one or more modification for increasing yield of the modified FGF-1 polypeptide from the cell. In some embodiments, the one or more modifications comprise sequence optimization for increased expression of the modified FGF-1 polypeptide in the cell. In one embodiment, the one or more modifications comprise modifications in the plasmid. In one embodiment, the one or more modifications comprise selecting a suitable promoter for increasing yield of the modified FGF-1 polypeptide from the cell.

In further embodiments, one or more modifications are considered towards developing the host cell for expressing the polypeptide. In some embodiments, one or more modifications may be considered resulting in adjusting the adequate nutrient media for maximizing cell proliferation. In one embodiment, the adequate nutrient media comprises a carbon source. In one embodiment, the carbon source is glucose or glycerol.

In one embodiment, one or more modifications are made in the plasmid to increase the copy number and expression efficiency of the plasmid in the host cell. In one embodiment, one or more modifications are considered for maximizing the yield of the modified FGF-1 polypeptide from the cell, wherein the one or more modifications may be selected from:
i. modification within the recombinant nucleic acid encoding the mutant FGF-1 polypeptide;
ii. modification within the recombinant nucleic acid comprising one or more regulatory elements related to the recombinant nucleic acid encoding the mutant FGF-1 polypeptide, selected from a promoter, an enhancer, a 5′-untranslated region, a 3′-untranslated region, a poly A tail, a transcript stabilizing element;
iii. modification of the plasmid comprising the recombinant nucleic acid;
iv. modification of the cell strain or selection of a cell strain for maximizing cell proliferation;
v. modification of the cell growth media; and
vi. modifications in the processes of recovering the modified FGF-1 polypeptide from the cell.

In some embodiments, the bacterial cells are electroporated or chemically transformed with a plasmid comprising a recombinant nucleic acid comprising a sequence encoding the FGF-1 polypeptide. Following the introduction of the foreign DNA, engineered cells may be allowed to grow for 1-2 days in an enriched media, and then are switched to a selective media. In some embodiments, one or more carbon sources are used for maximizing the bacterial cell growth within a period of time for expansion of the expressed FGF polypeptide for increased production. In some embodiments the carbon source for the bacterial cell may be glucose. In some embodiments the carbon source for the bacterial cell may be glycerol.

The selectable marker in the recombinant plasmid confers resistance to the selection and allows cells to stably integrate the plasmid into their chromosomes and grow to form foci that in turn can be cloned and expanded into cell lines. In some examples, this method may advantageously be used to engineer cell lines that express the modified FGF-1 polypeptide product. Such engineered cell lines may be particularly useful in screening and evaluation of compounds that affect the biological of the gene product.

The method of production described herein can be easily scaled up to form large scale productions of bacterial cultures expressing the modified FGF-1. In some embodiments, the method can be scaled to produce FGF-1 in 1 L bacterial cultures, or in 10 L bacterial cultures, or in 100 L bacterial cultures, or in 500 L bacterial cultures. In some embodiments, the method is scalable to produce 1 g of modified FGF-1 polypeptide per batch. In some embodiments, the method is scalable to produce 10 g of modified FGF-1 polypeptide per batch. In some embodiments, the method is scalable to produce 100 g of modified FGF-1 polypeptide per batch. In some embodiments, the method is scalable to produce 1 Kg of modified FGF-1 polypeptide per batch. In some embodiments, the method is scalable to produce 10 Kg of modified FGF-1 polypeptide per batch. In some embodiments, the method is scalable to produce 100 Kg of modified FGF-1 polypeptide per batch.

In general, a seed culture is formed from transformed bacterial cells. The inoculated seed flasks may be incubated at about 235 RPM and 37° C. Following a 10-14 h incubation, a sample of the seed culture from each of the flasks may be tested for purity (microscopic observation of a wet mount with no contaminating organisms observed), pH, optical density at 600 nm (OD₆₀₀), and sterility hold. The seed cultures are desired to exhibit optimal growth by demonstrating an OD₆₀₀≥1.0 and no contaminating organisms. Six of the seed flasks from each production run may be selected for scale-up. Selection criteria may include a growth time of 12±2 h, an OD₆₀₀≥1.0, and having six flasks. To create the fermentor inoculum, the contents of the six flasks may be pooled into a 10 L bag (sterile single-use bioprocess container) in a BSC for a total seed culture volume of approximately six liters.

One or more 150 L fermentors may be prepared for the fermentation of cultured E. coli expressing mFGF-1 with production medium (comprising nutrients, for example, soytone 12 g/L, yeast extract 24 g/L, glycerol 15.1 g/L, potassium phosphate dibasic 12.5 g/L, potassium phosphate monobasic 3.8 g/L, and P2000 antifoam 0.1 mL/L). The production medium is sterilized in situ. The sterile medium is then supplemented with a 5±0.1 L sterile solution that contained magnesium sulfate heptahydrate 0.4 g/L, kanamycin 0.050 g/L. The one or more than one fermentors may be inoculated with six liters of pooled seed culture at the appropriate time. Fermentation cultures are then monitored every 60±30 min and the samples were processed for pH, purity (microscopic observation of a wet mount), and OD₆₀₀. Dissolved oxygen may be maintained by controlling agitation and air flow rates. The pH may be maintained within the desired range by making appropriate aseptic additions of phosphoric acid and/or ammonium hydroxide.

In some embodiments, the expression of mFGF-1 induced with addition of 0.2-0.4 g/L isopropyl-3-D-1-thio-galactopyranoside (IPTG) and 5.0 g/L L-Arabinose when the culture reaches an OD₆₀₀ of ˜4.5 at 3 hours post-inoculation. In some embodiments, a concentration of about 1 mM or 0.25 mM IPTG is used for induction, with or without kanamycin. Induction in the presence of kanamycin, in some embodiments, is not required to increase plasmid retention and cell viability, but, affects the production of the modified FGF-1 polypeptide. The length of induction, in some instances, is from about 8-12 hours, about 10-20 hours, about 20 hours, or about 24 hours. Yield of the modified FGF-1 polypeptide (as measured by final OD/cell paste (g/L)) in the presence of kanamycin, in some instances at a 1 L scale is about 1.1 to about 5 times, e.g., 1.2 times, greater than in the absence of kanamycin. For induction, in some embodiments, the carbon source is glycerol, e.g., at a concentration of at least about 30 g/L, temperature is about 37° C., and pH about 6.8. After induction, the fermentations may be continued for an additional three hours. Prior to centrifugation samples may be taken from each fermentor for analysis by SDS-PAGE and culture purity. The fermentations may be evaluated intermittently and may be grown for another 1-20 hours. In some embodiments, the final culture reaches an optical density of 50-100, 50-200, 50-250, 70-260 or about 100, 200 or 250.

In some embodiments, the harvesting of the lots may be performed by transferring the fermentation broth to the centrifuge via a peristaltic pump and tubing (at 0.5-0.8 liters per minute) and centrifuged at 20,000×g while cooling using a water-circulation jacket. The mass of the harvested cell paste may be measured, collected, divided into four containers, and placed in a ≤−70° C. freezer.

Cell Lysis: Frozen cells comprising the modified FGF-1 may be thawed at a suitable time, and resuspended in a suitable buffer, for example, the buffer may comprise Tris and EDTA. In an exemplary embodiment, the cells may be thawed in TES buffer (50 mM Tris, 20 mM EDTA, 100 mM NaCl, pH7.4) containing 1 mM DTE at a ratio of 1:5 (w/v), i.e. 1 gram of cell paste in 5 mL of buffer. The suspension may be chilled to below 16° C. before running through a high-pressure homogenizer. OD₆₀₀ is monitored after each pass until no significant decrease. An equal volume of TES+5% Triton X-100 may then be mixed into the ruptured-cell suspension. The ruptured cell suspension may be used for recovery of the expressed FGF-1 proteins. The expressed protein may be collected from the ruptured cell by passing the lysate through a specific capture method, such as affinity column packed with an agarose based resin (e.g., a highly cross-linked agarose based resin, such as, Capto™ DeVirs (Cytiva, BPG 300×500, Part #17-5466)), that specifically binds the FGF-1 protein, and is later eluted. However, for maximizing the FGF-1 recovery and yield, the protein may be directed for expression in the IBs, and the protein may be collected from the inclusion bodies. In an exemplary method, the mixture may be centrifuged at 15,900×g for 60 min at 4° C. to collect the mFGF-1-containing inclusion bodies. Following lysis, the overexpressed proteins may be recovered from inclusion bodies (IB) from E. coli paste by centrifugation.

In some embodiments, the capture method using an affinity column packed with an agarose based resin (e.g., Capto™ DeVirs) increases percent yield of the collected FGF-1 polypeptide by about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%, in comparison to other capture methods.

Recovery from inclusion bodies: In an exemplary embodiment, for the recovery of FGF-1, the cell paste may be thawed at 2-8° C., resuspended in a suitable buffer. The buffer may comprise Tris and EDTA. For example, the cell paste may be thawed in 4.5 L of TES buffer, pH 7.4 (50 mM Tris, 100 mM NaCl, 20 mM EDTA) and the cells may then be lysed by pressure homogenization. Five homogenization passes (approximately 8,000 psi) may be performed to achieve maximum cell lysis. An equal volume of TES buffer and 5% Triton X-100, pH 7.4 may then be added to the lysate to obtain a 2.5% Triton concentration.

The mixture may be divided into 6-20 centrifuge bottles, as per convenience, which are then incubated for at least 30 min at approximately 15-20° C. with shaking at 225 RPM. The bottles may be centrifuged for 60 min at 15,900×g and 4° C. The supernatant was discarded as waste. Using a tissue homogenizer (Model Omni GLH850), the recovered inclusion bodies were individually resuspended in TE Buffer (˜1 L, 50 mM Tris, 20 mM EDTA, pH 7.4) with 2.5% (w/v) Triton X-100, and the bottles were incubated for at least 30 min at 15-20° C. with shaking at 225 RPM. After incubation, the bottles were centrifuged for 45 min at 15,900×g and 4° C. The inclusion body washing process suspension, incubation, and centrifugation was performed a total of three times. The recovered inclusion bodies may be stored overnight at 2° C.-8° C. The inclusion bodies were washed with TE buffer without Triton (˜1 L, 50 mM Tris, 20 mM EDTA, pH 7.4) and the bottles were incubated for at least 15 min at 15-20° C. with shaking at 225 RPM. In some embodiments, the IBs may be washed in a buffer comprising polysorbate 20 or polysorbate 80. After incubation, the bottles were centrifuged for 30 min at 15,900×g and 4° C. Washing without Triton, incubation, and centrifugation may be performed a total of five times. Samples may be removed and submitted for total protein and SDS-PAGE Coomassie Stain/densitometry analysis at this stage. In some embodiments, a total of 100, 200, 300, 400, 500, 600, 700, 800, 900 or 1,000 g of inclusion bodies may be recovered. The centrifuge bottles containing the washed inclusion bodies for each lot may be stored at ≤−70° C.

Solubilization of Washed Inclusion Bodies: Solubilization of the washed inclusion bodies for each lot may be performed using a solubilization buffer. A solubilization buffer may comprise chaotropic components, such as urea, or guanidine salts. The washed inclusion bodies may be removed from storage and thawed at 2-8° C. (15 h-19 h). Inclusion bodies may be centrifuged at 15,900×G, 4° C. for 60 min, and after removing the liquid phase the net weight of the pellets was determined. The inclusion bodies may then be solubilized in buffer. An exemplary solubilization buffer may comprise 4-8M Guanidine. An exemplary solubilization buffer may comprise 6M Guanidine. An exemplary solubilization buffer may comprise 4-6M Urea. Additionally, such a buffer may comprise 100 mM Tris, 2 mM EDTA, (pH 8.0). The buffer may be mixed at 10 mL per g ratio at 2-8° C. using a tissue homogenizer (e.g., Model Omni GLH850) at 10,000 RPM until the solution was visually homogenous. Guanidine is a chaotropic agent that results on protein denaturation. Dithioerythritol (DTE) may be used to a final concentration of 10 mg/mL to the solubilized inclusion bodies at 2-8° C. to reduce disulfide bonds to thiols. This reaction may continue for 2-6 hours. The solubilized inclusion bodies may then be centrifuged for 40 min at 15,900×g and 4° C. The complete process including centrifugation may be less than five hours. The supernatant may be collected into 2 L PETG bottles and stored at 2-8° C. (25-50 min) while the protein concentration was tested. Based on the protein results, the solubilized inclusion bodies may be diluted to a target concentration of 2.0±0.5 mg/mL with dilution buffer (6M Guanidine, 100 mM Tris, 2 mM EDTA, pH 8.0). DTE may be added to a target concentration of 10 mg/mL and mixed. The solubilized inclusion bodies may be stored at ≤−70° C. The IBs may be solubilized in 6 M guanidine hydrochloride in 100 mM Tris, 2 mM EDTA, pH 8.0 at a ratio of 10 mL buffer per gram IB. DTE may be added (10 mg/ml) and after 3-5 h mixing (initially with tissue homogenizer, Polytron PT 3100, followed by magnetic stir bar), the mixture may be centrifuged (15,900×g≥40 min). The supernatant may be filtered through a 0.45 μm filter.

Refolding of denatured protein: Guanidine-solubilized IBs (2±0.5 mg/mL) may be added into cold refolding buffer. The refolding buffer may comprise L-arginine. (e.g., 0.5 M L-Arginine, 100 mM Tris, 2 mM EDTA, pH 9.5). In some embodiments the refolding buffer may contain oxidized glutathione. In some embodiments, the refolding buffer may contain reduced glutathione. The solubilized mFGF-1 may be added slowly, e.g., dropwise into the vortex of the refold solution and mixing continued at 2-8° C. for 2 h. An equal volume of 3M ammonium sulfate may be added to the refolding solution and stirred at 2-8° C. for 1 h.

The recovered protein may be detected by SDS-PAGE. Total protein content may be measured by one or more methods known in the art. For example, total protein content may be measured by Coomassie stain of proteins resolved in SDS-PAGE. The recovered protein may be further purified using HPLC, for example size exclusion chromatography (SEC)-HPLC.

The biological activity of the protein may be assessed by cell proliferation assay in vitro. For this purpose, endothelial cell lines may be used. In some embodiments, fibroblasts may be used for in vitro proliferation assay.

In some embodiments one or more modifications are made to improve the recovery yield of the modified FGF-1 from the cell. The one or more improvements comprise improvements of the plasmid vector; improvement in choice of a suitable bacterial strain; improvement in the growth media; improvement in induction time with IPTG; improvement in incubation time for maximal growth of the bacteria; and optimizing the temperature for growth of the bacteria. In some embodiments, the one or more modifications lead to at least 2 fold, at least 3 fold, at least 4 fold, to at least 6 fold, at least 7 fold, at least 8 fold, to at least 9 fold, at least 10 fold, at least 12 fold, at least 15 fold, to at least 20 fold, at least 25 fold, at least 30 fold or at least 50 fold increase in the yield of modified FGF-1.

### Disulfide Bond Formation in Modified FGF-1 Polypeptides

In some embodiments, the modified FGF-1 polypeptide of the present disclosure comprises the following mutations in SEQ ID NO: 1-Cys16Ser, Ala66Cys, and Cys 117Val, wherein the polypeptide includes an internal disulfide bond between the cysteine residues at positions 66 and 83. For many recombinant proteins, the formation of correct disulfide bonds is vital for attaining their biologically active three-dimensional conformation. The formation of erroneous disulfide bonds can lead to protein misfolding and aggregation into inclusion bodies. In E. coli, cysteine oxidation typically takes places in the periplasm, where disulfide bonds are formed in disulfide exchange reactions catalyzed by a myriad of enzymes, mainly from the Dsb family (Rosano, G. L., & Ceccarelli, E. A. (2014). Recombinant protein expression in Escherichia coli: advances and challenges. Frontiers in Microbiology, 5, 172). By contrast, disulfide bond formation in the cytoplasm is rare. This situation affects the production of recombinant proteins with disulfide bonds that are produced in the cytoplasm, such as a modified FGF-1 polypeptide comprising an internal disulfide linkage between Cys66 and Cys83. Accordingly, in some examples, an engineered E. coli strain that possess an oxidative cytoplasmic environment that favors disulfide bond formation is selected as a host cell for expression of the modified FGF-1 polypeptides (Rosano, G. L., & Ceccarelli, E. A. (2014). Recombinant protein expression in Escherichia coli: advances and challenges. Frontiers in Microbiology, 5, 172). Examples of such strains include but are not limited to Origami (Novagen), which has a trxB-gor-genotype in the K-12 background, and SHuffle® T7 Express strain (NEB), which has a trxB-gor-genotype in a BL21(DE3) background and constitutively expresses a chromosomal copy of the disulfide bond isomerase DsbC. It has been shown that DsbC promotes the correction of mis-oxidized proteins into their correct form and is also a chaperone that can assist in the folding of proteins that do not require disulfide bonds. Without being bound by a particular theory, it is contemplated that due to the action of DsbC, less target protein, such as the modified FGF-1 polypeptide comprising an internal disulfide linkage between Cys66 and Cys83, aggregates into inclusion bodies. Thus, in certain embodiments, the present disclosure identifies an improved method for cytoplasmic production of a modified FGF-1 polypeptide comprising internal disulfide linkage between Cys16 and Cys83.

In some embodiments where the modified FGF-1 polypeptide is expressed with an N-Met residue, the polypeptide is subsequently purified without a step requiring proteolytic cleavage for removal of an N-terminal peptide. Accordingly, in some embodiments, the present disclosure provides a method of rapid purification of the modified FGF-1 polypeptides described herein, without involving a proteolytic cleavage step for removal of an N-terminal peptide. This is particularly advantageous for production of the modified FGF-1 polypeptides per good manufacturing practice (GMP) guidelines. The advantages include the lack of a cleavage step, including eliminating the need for subsequent purification of the cleaved product and removal of the reagents used for cleavage. The further advantage of this is an increase in yield due to decreased handling and the alleviation of the need to test for residual cleavage reagents and contaminants introduced for the cleavage and subsequent separation of cleaved from uncleaved material.

### Methods of Use

Provided herein, in one embodiment, is a method of treating an ocular disease, disorder or condition in a mammal comprising administering to the mammal a modified FGF-1 polypeptide as described in the above embodiments or a pharmaceutical formulation comprising the same, as described herein. In some instances, the modified FGF-1 polypeptide for use in the methods described herein comprises a sequence selected from SEQ ID NOs: 2, and 9-204. Provided herein, in one embodiment, is a method of treating an ocular disease, disorder or condition in a mammal comprising administering to the mammal a modified FGF-1 polypeptide comprising a sequence as set forth in SEQ ID NO: 205 or 206, or a pharmaceutical formulation comprising the same as described herein.

In some embodiments, the ocular disease, disorder or condition to be treated is a disease, disorder, or condition of the corneal endothelial layer. Diseases, disorders, or conditions of the corneal endothelial layer include, but are not limited to, Fuch's dystrophy, bullous keratopathy, congenital hereditary endothelial dystrophy 1, congenital hereditary endothelial dystrophy 2, and posterior polymorphous corneal dystrophy.

Without being bound by theory, it is believed a solution (e.g., a pharmaceutical formulation as described herein) of a modified FGF-1 polypeptide injected intracamerally into the aqueous humor of the eye binds to the endothelial surface and especially any areas of the cornea that are not covered by a healthy endothelial layer. The modified FGF stimulates the growth and migration of the endothelial cells. This reduces the corneal edema associated with the endothelial dystrophy and reduces the likelihood for a need for a corneal or endothelial transplant. The action of the modified FGF can occur at a site other than the site of greatest dystrophy (typically at the corneal center) and also results in stimulation of endothelial cells in the corneal periphery and endothelial progenitor pools in the trabecular Meshwork™.

In some embodiments, the ocular disease, disorder or condition to be treated is a disease, disorder, or condition of the corneal epithelium. Diseases, disorders or conditions of the corneal epithelium include, but are not limited to, recurrent corneal erosions, persistent epithelial defects, dry eye syndromes, inflammatory conditions such as Stevens-Johnson syndrome, and corneal epithelial defects.

In some embodiments, the ocular disease, disorder or condition to be treated is herpetic keratopathy. Herpetic keratopathy typically is an infection of the cornea caused by Herpes Simplex virus (HSV). Primary infection can be the result of direct exposure of the host's mucous membranes to infectious HSV. Following primary infection and the establishment of latency in the sensory ganglia, the virus can be stimulated to enter an infectious cycle, from which it returns to the cornea. Once there, this recurrent infection can cause various complications, in particular an inflammatory response, which if strong enough can compromise the integrity of the cornea, leading to corneal ulcer, opacity, haze, scarring and in severe cases blindness. Secondary to herpes infection, there can be development of chronic herpetic keratopathy, neurotrophic keratopathy, or both. For example, stromal infections, which are immune-mediated and are the leading cause of corneal blindness in developed countries occur as a result of chronic viral reactivation, and lead to neurotrophic keratopathy, a degenerative condition. A normal cornea is densely innervated, but lacks blood vessels. Subsequent episodes following primary viral infection can not only damage nerves, leading to decreased corneal sensation (corneal hypoesthesia), but also lead to angiogenesis, and neovascularization. In further embodiments, the modified FGF-1 polypeptides described herein can be used to treat infectious keratitis caused by bacterial or fungal infections.

In further embodiments, the modified FGF-1 polypeptides described herein can be used to treat epithelial basement membrane dystrophy, Meesmann juvenile epithelial corneal dystrophy, gelatinous drop-like corneal dystrophy, Lisch epithelial corneal dystrophy, subepithelial mucinous corneal dystrophy, Reis-Bucklers corneal dystrophy, or Thiel-Behnke dystrophy, and recurrent corneal erosions.

In some embodiments, the ocular condition includes damage to the cornea (e.g., the corneal surface or endothelial layer at the interface of the cornea and aqueous humor) or surgical disruption caused by corneal surgeries, including PRK, LASIK, and any penetrating corneal surgery or keratoplasty.

Also provided herein is a method of treating a chemical or vesicant agent induced injury by administering a modified fibroblast growth factors (FGF-1) polypeptides, or pharmaceutical composition or medicaments that include such modified peptides.

Also provided herein in one embodiment is a method of treating a chemical or vesicant injury by administering a modified FGF-1 polypeptide as described herein. In some embodiments, the method comprises treating a skin injury or an ocular injury caused by a chemical or a vesicant agent. In some embodiments, the method comprises treating mustard gas keratopathy, induced by a vesicant, e.g., nitrogen mustard (NM), by administering modified FGF-1 as polypeptides described herein. Treating MGK with a modified FGF-1 polypeptide, as described herein, in some embodiments, results in amelioration of histopathological conditions associated with MGK, such as hyperplasia of corneal epithelial layer, epithelial-stromal cell separation edema, corneal erosions. The administration of modified FGF-1 of the present disclosure, in certain embodiments, results in reduction in edema and elimination of corneal erosions. Corneal erosion is typically characterized by de-epithelialization of the cornea and in some examples; administration of the modified FGF-1 results in faster re-epithelialization of the cornea or reduces the severity of corneal de-epithelialization. In one embodiment is described a method of regenerating ocular surface epithelium in a patient exposed to a chemical or a vesicant, by administering a modified FGF-1 as described herein. In some embodiments, the method promotes regeneration of cornea, prevents degeneration of the cornea, and prevents long term sequelae to the chemical injury. In some examples, the method comprises treating a corneal endothelial injury, a corneal epithelial injury, or a corneal stromal injury. In instances where the method treats corneal endothelial injuries, administering a modified FGF-1, as described herein, enhances the function of corneal endothelial cells and prevents long term degeneration of the cornea. In some instances, where the method treats corneal endothelial injuries, administering a modified FGF-1, as described herein, prevents corneal edema and secondary anterior keratopathies. In some instances, where the method treats corneal endothelial injuries, administering a modified FGF-1, as described herein, prevents loss of corneal endothelial cells. In some embodiments, the method results in reduction of the severity of corneal epithelial detachment. In some embodiments, the method comprises treating a stromal injury such as stromal scarring and corneal opacity.

In some embodiments, the ocular condition includes accidental trauma or chemical or thermal injury to the cornea. In some examples, the chemical or thermal injury is a chemical burn. In some examples, the chemical or thermal injury is caused by a vesicant agent. In some examples, the chemical or thermal injury is caused by a chemical warfare agent.

A multitude of household and occupational compounds have the potential to induce chemical burns to the eye and skin. Without prompt intervention, irreversible visual loss and disfigurement may prevail. Agents that rapidly neutralize both acid and alkali agents without heat release and limit diffusion, are contemplated to be effective in treating chemical injuries. Exemplary chemical injuries include, but are not limited to, alkali injuries, acid injuries. Common sources of chemical burns include sulfuric acid (H₂SO₄), hydrochloric acid (HCl), sodium hydroxide (NaOH), lime (CaO), silver nitrate (AgNO₃), hydrogen peroxide (H₂O₂), chlorine gas and any strong oxidant.

Exemplary chemical warfare agent that can cause the chemical or thermal injury described herein, is phosgene, an urticant, or a nettle agent. Phosgene is a highly toxic, colorless gas at room temperature and standard pressure that condenses at 0° C. to a fuming liquid. Its molecular formula is COCl₂. Phosgene is extremely toxic by acute (short-term) inhalation exposure. Severe respiratory effects, including pulmonary edema, pulmonary emphysema, and death have been reported in humans. Severe ocular irritation and dermal burns may result following eye or skin exposure. Chronic (long-term) inhalation exposure to phosgene may also cause irreversible pulmonary changes, such as emphysema and fibrosis. Its exposure can result in widespread and devastating effects including high mortality due to its fast penetration and ability to cause immediate severe cutaneous injury. Results from a recent study show that topical cutaneous exposure to phosgene vapor causes blanching of exposed skin with an erythematous ring, necrosis, edema, mild urticaria and erythema within minutes after exposure out to 8 h post-exposure, in a mouse model. These clinical skin manifestations are accompanied with increases in skin thickness, apoptotic cell death, mast cell degranulation, myeloperoxidase activity indicating neutrophil infiltration, p53 phosphorylation and accumulation, and an increase in COX-2 and TNFα levels. Topical phosgene-exposure also resulted in the dilatation of the peripheral vessels with a robust increase in RBCs in vessels of the liver, spleen, kidney, lungs and heart tissues. It is contemplated that these events could cause a drop in blood pressure leading to shock, hypoxia and death. See, Tewari-Singh N, Goswami D G, Kant R, Croutch C R, Casillas R P, Orlicky D J, Agarwal R, Cutaneous exposure to vesicant phosgene oxime: Acute effects on the skin and systemic toxicity, Toxicol Appl Pharmacol. 2017 Feb. 15; 317:25-32.

In some embodiments, the modified FGF-1 polypeptide may be used in a method of treating, preventing, or ameliorating the various skin injuries caused by vesicant exposure.

Vesicants, or vesicant agents, or blistering agents are toxic compounds that produce skin injuries resembling those caused by burns. These agents on inhalation affect the upper respiratory tract as well as the lungs, producing pulmonary edema. See, e.g., Ganesan, K., S. K. Raza, and R. Vijayaraghavan (2010) Chemical Warfare Agents, Journal of Pharmacy and Bioallied Sciences 2.3: 166-178. These agents can also cause severe eye injuries. There are two forms of vesicants: mustards and arsenicals. The most important substance in this class of chemical warfare agents is sulfur mustard. Other members include nitrogen mustards (HN1, HN2 and HN3), and arsenic vesicants such as lewisites (L1, L2 and L3), ethyldichloroarsine, methyldichloroarsine, phenyldichloriarsine. Specific examples of vesicant agents include but are not limited to sulfur mustard (SM), bis-(2-chloroethyl) sulfide, chloroethylethyl sulfide (CEES), lewisite, and 2-chloro-N-(2-chloroethyl)-N-methylethanamine hydrochloride, a member of the family of nitrogen mustard (NM). As used throughout this disclosure, the terms vesicant, vesication-causing agent or chemical, vesicating agent, and the like, are taken to mean vesicants as specifically enumerated herein, and other compounds, such as toxins and/or chemical warfare agents. Sulfur mustard is the vesicant with the highest military significance since its use in WWI. The nitrogen mustards were synthesized in the 1930s but were not produced in large amounts for warfare. Mechlorethamine (HN2, Mustargen) has found more peaceful applications as a cancer chemotherapeutic agent and has remained the standard compound for this purpose for many years. Lewisite (L) was synthesized in 1918 for military purpose due to its non-flammable property and toxicity similar to mustard but has probably not been used on a battlefield. The mustards are radiomimetic and are extremely toxic to dividing cells. Mustards are lipophilic and readily penetrate the skin, most textiles and rubber. After passing through the cellular membrane, sulfur mustard is converted to highly reactive sulphonium ion. It irreversibly alkylates DNA, RNA and protein, causing cell death; the most important target is DNA. Mustard alkylates the purine bases of DNA and damages them. Lewisite is absorbed by the skin much faster, and it causes immediate pain and irritation in the affected organ and produces more systematic symptoms. It directly binds to the sulfhydryl groups and inactivates them.

The use of sulfur mustard (SM), and other vesicating agents in chemical warfare has been long known. More recently, in August 2015, SM was used by ISIS in an attack on Kurdish forces in Iraq, as well as an attack in Syria. Mustard agents injure the eyes, the skin, and the lungs, with the eyes being the most sensitive. Because symptoms do not manifest until 2 to 4 hours after exposure, exposed persons do not immediately know they are exposed to mustard. This delay has contributed to confusion and panic when symptoms of exposure finally develop. For the eyes, these consist of blepharospasm, lacrimation, irritation, pain, and photophobia. Corneal injuries resulting from ocular exposure to sulfur mustard (SM) vapor are the most prevalent chemical warfare injury. Ocular exposures exhibit three distinct, dose-dependent clinical trajectories: complete injury resolution, immediate transition to a chronic injury, or apparent recovery followed by the subsequent development of persistent ocular manifestations. These latter two trajectories include a constellation of corneal symptoms that are collectively known as mustard gas keratopathy (MGK). Tissue-specific damage during the acute injury can decrement the regenerative capacities of corneal endothelium and limbal stem cells, thereby predisposing the cornea to the chronic or delayed forms of MGK.

For some patients MGK occurs a few weeks after exposure; in others it took years to manifest. This keratopathy is characterized by corneal conjunctivalization and limbal stem cell deficiency. It has been shown that in the human corneal endothelium, gaps due to CEC loss are typically filled by spreading of proximal CECs. These morphological changes compensate for endothelial loss until the barrier between the cornea and aqueous humor can no longer be maintained, resulting in persistent corneal edema and secondary anterior keratopathies. Because adult human CECs do not proliferate in vivo, any loss of CECs therefore potentially represents a permanent reduction in endothelial capacity. Thus, while endothelial function can be restored after a mild injury by CEC spreading, more severe injuries may exceed the repair capacity of the human endothelium. Rabbits are distinct from humans in that they can undergo limited CEC proliferation, giving them an improved capacity to recover from CEC loss. However, as in humans, sufficiently severe injury to the rabbit endothelium also results in irreversible corneal decompensation and secondary keratopathies.

Based on the above studies, it has been hypothesized that vesicant-induced endothelial failure may be the causal mechanism underlying MGK pathogenesis. This hypothesis is consistent with the dose dependence between SM and the development of MGK that has been observed in humans and rabbits, as well as the different clinical trajectories (resolved chronic MGK and delayed-onset MGK) that have been reported in human casualties. According to this hypothesis, cornea exposure to low doses of vesicant may result in an acute epithelial lesion, with minimal endothelial toxicity, and corneas recover without long-term complications. Alternatively, exposure to doses of a vesicant that cause irreparable injury to the corneal endothelium could result in endothelial barrier failure, producing a persistent edema with secondary anterior keratopathies. Following a severe injury, there may be no apparent delay between the acute injury and MGK onset. Hence, a composition and method for minimizing or preventing injury due to sulfur mustard and similarly acting chemical toxicants, particularly chemical warfare agents, is an important pursuit for scientists working for the U.S. Department of Defense. Recent studies have shown that as vesicating agents, mustard compounds lead to a loss of epithelial-stromal attachment. In the cornea, microbullae are formed, and once enough have accrued, the corneal epithelium is unable to hold fast to the basement membrane, causing the epithelial tissue to slough. Thus, an effective post-exposure therapy for SM is desired to enhance the ability of the corneal epithelium to remain attached to the stroma. Without being bound by a theory, it is contemplated that the ability of the corneal epithelium to remain attached to the stroma might allow some basal epithelia the opportunity to recover in situ, maintaining their connections with their basement membrane and stroma. It has also been hypothesized that one of the key players in the epithelial-stromal integrity is collagen XVII (i.e., BP180), a transmembranous component of the hemidesmosome. Cleavage of collagen XVII by ADAM (“A Disintegrin And Metalloproteinase”) family of proteins, including ADAM9, ADAM10, and/or ADAM17 after injury releases epithelial cells from their basement membrane, and this cleavage allows them to migrate.

ADAM17, also known as TNF-α converting enzyme or TACE, is a general response to injury as well as a “sheddase” for releasing collagen XVII. It was postulated that corneal microblistering, induced by vesicant agent exposure, is in part due to activation of ADAM17, which is capable of cleaving collagen XVII. Experimental data confirmed the induction of ADAM17 expression at the basement membrane zone of corneas exposed to vesicant agent NM. Thus, agents that are able to inhibit the post-exposure upregulation of ADAM17 expression are contemplated to be useful for attenuation of corneal injuries caused by vesicant agents.

The present disclosure provides modified FGF-1 polypeptides that treat, reduce the adverse effects or, and otherwise aid in the healing of exposure to vesicant agents, such as SM and NM. The modified FGF-1 polypeptides disclosed herein are capable of preventing the overexpression of ADAM17 following exposure to a vesicant agent, such as SM and/or NM.

The present disclosure also provides a method of treating, preventing, reducing the adverse effects of, and otherwise aiding the healing of exposure to chemical or vesicant induced injury, by administering a modified FGF-1 polypeptide. In some embodiments, the methods disclosed herein further prevent the overexpression of ADAM17 following exposure to a vesicant agent, such as SM and/or NM.

Wild type FGF-1 proteins, e.g., SEQ ID NO: 1, which have unpaired cysteine residues that are susceptible to oxidation and alkylation. In some embodiments of the present disclosure where the modified FGF-1 polypeptides do not comprise unpaired cysteine residues, such modified FGF-1 polypeptides are less susceptible to oxidation and/or alkylation by vesicant agents. Experimental data has also indicated reduction in levels of FGF-1 and its mRNA are known to result from exposure to mustard agents and it is hypothesized that this loss may play a role in the slow healing of mustard-induced lesions in the cornea. In some embodiments of the present disclosure, the modified FGF-1 polypeptides, which do not comprise free cysteine residues and accordingly are less or not susceptible to cysteine modification, are effective in accelerating the healing of corneal mustard lesions.

In some embodiments of the present disclosure, the method comprises administering a modified FGF-1 polypeptide that does not comprise unpaired cysteine residues, which modified FGF-1 polypeptide is less susceptible to oxidation and/or alkylation by vesicant agents. In some embodiments of the present disclosure, the method comprises administering a modified FGF-1 polypeptide, which does not comprise free cysteine residues and accordingly is less or not susceptible to cysteine modification. In some embodiments, the method disclosed herein is effective in accelerating the healing of corneal lesions associated with MGK.

Exposure to vesicant agents, such as sulfur mustard (SM) and nitrogen mustard (NM) can cause severe skin injury with delayed blistering. Depending upon the dose and time of their exposure, edema and erythema can potentially develop into blisters, ulceration, necrosis, desquamation, and pigmentation changes, which persist weeks and even years after exposure. See, e.g., Tewari-Singh N, Agarwal R, Mustard vesicating agent-induced toxicity in the skin tissue and silibinin as a potential countermeasure, Ann N Y Acad Sci. 2016 June; 1374(1):184-92. Another exemplary vesicant agent Phosgene Oxime (CX), an urticant or nettle agent, is also a potential chemical warfare and terrorist weapon.

In some embodiments, the ocular disease, disorder or condition to be treated is a disease, disorder, or condition of the corneal stroma. Diseases, disorders or conditions of the corneal stroma include, but are not limited to, keratoconus, lattice corneal dystrophy, granular corneal dystrophy, macular corneal dystrophy, Schnyder crystalline corneal dystrophy, congenital stromal corneal dystrophy, fleck corneal dystrophy, trauma or chemical or thermal injury, or injury secondary to infections such as trachoma.

In further embodiments, the modified FGF-1 polypeptides described herein can be applied before, during, or after corneal transplantations procedures (e.g., corneal transplantation or procedures involving Descemet's membrane) that involve disruption of the cornea (e.g., corneal endothelial structure) where acceleration of healing of corneal or ocular surface cells and/or improving the cellular response (e.g., by increasing the viability and/or longevity of the transplanted cells) to insult would result in a therapeutic benefit.

In additional embodiments, the modified FGF-1 polypeptides described herein can be used to increase the viability and health of corneal cells or corneal progenitors being prepared for transplantation. Modified FGF-1 polypeptides added to the organ culture medium for donated corneas or other donated corneal tissue stimulates the corneal cells and increases the length of time the corneas can be stored before transplantation, as well as increasing the probability that a cornea will have sufficient healthy cells to be useful for transplantation. Also, the modified FGF-1 polypeptides can be used in culture media when culturing corneal progenitor cells to stimulate growth of those cells.

Further embodiments relate to methods of modulating the activity of one or more fibroblast growth factor receptors (FGFRs) in a corneal endothelial cell comprising contacting said corneal endothelial cell with a modified FGF (e.g., a modified FGF-1, such as one comprising the sequence of SEQ ID NO: 2). Such methods can be used to increase or stimulate the activity of one or more FGFRs, which can result in increased cell migration and/or cell proliferation.

In additional embodiments are described methods of treating a metabolic disease by administering a modified FGF-1 polypeptide according to the present disclosure. Exemplary metabolic diseases that can be treated with the disclosed modified FGF-1 polypeptides include but are not limited to: (1) glucose utilization disorders and the sequelae associated therewith, including diabetes mellitus (Type I and Type-2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic 3-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thromboses, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinflammatory processes and/or other peripheral neuropathies, including Alzheimer's disease, multiple sclerosis, Parkinson's disease, progressive multifocal leukoencephalopathy and Guillian-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Also described are methods of reducing fed and fasting blood glucose, improving insulin sensitivity and glucose tolerance, reducing systemic chronic inflammation, ameliorating hepatic steatosis in a mammal, reducing food intake, or combinations thereof, by administering a therapeutically effective amount of a disclosed modified FGF-1 polypeptide (or nucleic acid molecules encoding such).

In some embodiments, the modified FGF-1 polypeptides are administered for wound healing. Examples of wounds include, but are not limited to, abrasions, avulsions, blowing wounds (e.g., open pneumothorax), burn wounds, contusions, gunshot wounds, incised wounds, open wounds, penetrating wounds, perforating wounds, puncture wounds, seton wounds, stab wounds, surgical wounds, subcutaneous wounds, diabetic lesions, or tangential wounds. Additional examples of wounds that can be treated by the compounds and compositions described herein include acute conditions or wounds, such as thermal burns, chemical burns, radiation burns, burns caused by excess exposure to ultraviolet radiation (e.g., sunburn); damage to bodily tissues, such as the perineum as a result of labor and childbirth; injuries sustained during medical procedures, such as episiotomies; trauma-induced injuries including cuts, incisions, excoriations; injuries sustained from accidents; post-surgical injuries, as well as chronic conditions, such as pressure sores, bedsores, conditions related to diabetes and poor circulation, and all types of acne. In addition, the wound can include dermatitis, such as impetigo, intertrigo, folliculitis and eczema, wounds following dental surgery; periodontal disease; wounds following trauma; and tumor-associated wounds. Yet other examples of wounds include animal bites, arterial disease, insect stings and bites, bone infections, compromised skin/muscle grafts, gangrene, skin tears or lacerations, skin aging, surgical incisions, including slow or non-healing surgical wounds, intracerebral hemorrhage, aneurysm, dermal asthenia, and post-operation infections.

A therapeutic peptide of the present invention may also be used to treat external wounds caused by, but not limited to scrapes, cuts, lacerated wounds, bite wounds, bullet wounds, stab wounds, burn wounds, sun burns, chemical burns, surgical wounds, bed sores, radiation injuries, all kinds of acute and chronic wounds, wounds or lesions created by cosmetic skin procedures. The peptide may also be used to ameliorate the effects of skin aging. The peptide may accelerate wound healing in an external wound and/or improve the cosmetic appearance of wounded areas, or skin subject to aging and disease. The peptide may be used to treat internal injury caused by, but not limited to, disease, surgery, gunshots, stabbing, accidents, infarcts, ischemic injuries, to organs and tissues including but not limited to heart, bone, brain, spinal cord, retina, peripheral nerves and other tissues and organs commonly subject to acute and chronic injury, disease, congenital and developmental malformation and aging processes.

In some embodiments, the modified FGF-1 polypeptides are administered for treating burn injury. Exemplary burn wounds include, but are not limited to, “burn ulcers” including, for example, ulceration that occur as a result of a burn injury, including a first degree burn (i.e., superficial, reddened area of skin); a second degree burn (a blistered injury site which may heal spontaneously after the blister fluid has been removed); a third degree burn (burn through the entire skin and usually require surgical intervention for wound healing); scalding (may occur from scalding hot water, grease or radiator fluid); a thermal burn (may occur from flames, usually deep burns); a chemical burn (may come from acid and alkali, usually deep burns); an electrical burn (either low voltage around a house or high voltage at work); an explosion flash (usually superficial injuries); and contact burns (usually deep and may occur from muffler tail pipes, hot irons, and stoves). As used herein, a delayed or difficult to heal wound may include, for example, a wound that is characterized at least in part by 1) a prolonged inflammatory phase, 2) a slow forming extracellular matrix (ECM), and 3) a decreased rate of epithelialization.

It has been shown that growth factors, e.g. FGF-1, play an important role in nerve regeneration and nerve healing. FGF-1 has been suggested for use in regenerating nervous system tissue following spinal cord injury or trauma, such as brachial plexus injury, neuroimmunologic disorders, such as acute or idiopathic transverse Myelitis™, or any other disease or condition where regeneration and/or protection of neurons or neural tissue is desired, since FGF-1 is believed to stimulate neural proliferation and growth and may be neuroprotective. See, e.g., Cheng, H. et al., “Spinal Cord Repair with Acidic Fibroblast Growth Factor as a Treatment for a Patient with Chronic Paraplegia,” SPINE 29(14):E284-E288 (2004); and Lin, P-H., “Functional recovery of chronic complete idiopathic transverse myelitis after administration of neurotrophic factors,” Spinal Cord 44:254-257 (2006). FGF-1 is known to have a neurotrophic activity, promote axonal growth, and exert beneficial effects in models of spinal cord injury and axon regeneration. Accordingly, in some embodiments the modified FGF-1 polypeptide of the present disclosure promotes neural regeneration and can be used in methods of treating conditions that benefit from neural regeneration. In some example methods, the neurological condition is amyotrophic lateral sclerosis (ALS). In some example methods, the neurological condition is acute or idiopathic transverse Myelitis™. In certain instances, the modified FGF-1 polypeptide can be administered in combination with other growth factors, as well as other pharmaceutically active components, for treating conditions that benefit from neural; regeneration.

### Pharmaceutical Compositions, Methods of Administration, and Dosing

Pharmaceutical compositions comprising a modified FGF-polypeptide as described herein may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

A pharmaceutical composition, as used herein, refers to a mixture of a modified FGF with other chemical components, such as carriers, stabilizers, diluents, dispersing agents, suspending agents, thickening agents, and/or excipients, and, optionally, other therapeutic and/or prophylactic ingredients. The pharmaceutical composition facilitates administration of the modified FGF to an organism. In practicing the methods of treatment or use provided herein, therapeutically effective amounts of modified FGF-1 polypeptides described herein are administered in a pharmaceutical composition to a mammal having an ocular disease, disorder, or condition to be treated. In some embodiments, the mammal is a human. A therapeutically effective amount can vary widely depending on the severity of the disease, the age and relative health of the subject, the potency of the compound used and other factors. A pharmaceutically acceptable or suitable composition includes an ophthalmologically suitable or acceptable composition.

A pharmaceutical composition (e.g., for delivery by injection or for application as an eye drop) may be in the form of a liquid or solid. A liquid pharmaceutical composition may include, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. Physiological saline is commonly used as an excipient, and an injectable pharmaceutical composition or a composition that is delivered ocularly (for example, as an eye drop) is preferably sterile.

A modified FGF-polypeptide or pharmaceutical composition described herein can be delivered to a subject by any suitable means, including, for example, topically, intraocularly, intracamerally, orally, parenterally, intravenously, intraperitoneally, intranasally (or other delivery methods to the mucous membranes, for example, of the nose, throat, and bronchial tubes), or by local administration to the eye, or by an intraocular or periocular device. Modes of local administration can include, for example, topical application, eye drops, intraocular injection or periocular injection. Periocular injection typically involves injection of the compound under the conjunctiva or into the Tennon's space (beneath the fibrous tissue overlying the eye). Intraocular injection typically involves injection of the modified FGF or pharmaceutical composition into the vitreous. In certain embodiments, the administration is non-invasive, such as by topical application or eye drops. In some embodiments, the administration is via a combination of topical and intracameral method.

A modified FGF or pharmaceutical composition thereof described herein can be formulated for administration using pharmaceutically acceptable (suitable) carriers or vehicles as well as techniques routinely used in the art. A pharmaceutically acceptable or suitable carrier includes an ophthalmologically suitable or acceptable carrier. A carrier is selected according to the solubility of the particular modified FGF. Suitable ophthalmological compositions and formulations include those that are administrable locally to the eye, such as by eye drops, injection or the like. In the case of eye drops, the formulation can also optionally include, for example, ophthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyethylene sorbitan mono-laurate (also referred to as Polysorbate 20), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

For injection, the modified FGF or pharmaceutical composition can be provided in an injection grade saline solution, in the form of an injectable liposome solution, slow-release polymer system or the like. Intraocular and periocular injections are known to those skilled in the art and are described in numerous publications including, for example, Spaeth, Ed., Ophthalmic Surgery: Principles of Practice, W. B. Sanders Co., Philadelphia, Pa., 85-87, 1990.

In some embodiments, the modified FGF or pharmaceutical composition (e.g., an ophthalmic formulation) is administered via microneedles into the cornea (Jiang et al. (2007). Invest Ophthalmol Vis Sci 48(9): 4038-4043). A microneedle array is coated with the modified FGF or pharmaceutical composition and pressed against the cornea such that the microneedles penetrate into the corneal stroma but do not penetrate the entire cornea. It is then removed, and the modified FGF or pharmaceutical composition is left behind in the corneal stroma. This modified FGF or pharmaceutical composition can stimulate the corneal cells to proliferate and migrate, and suppresses the scarring response that the stromal cells normally have.

In some embodiments, the composition may be formulated for intraocular delivery. Intraocular delivery comprises intravitreal delivery, corneal injections, intracameral delivery. In some embodiment the composition is formulated for intracameral delivery. In some embodiments the composition is formulated for intravitreal delivery. The formulation is an injectable liquid, may comprise a very small volume, and the density of the injectable liquid formulation may be adjusted such its release in the targeted space does not incur injury to the tissue. In some embodiments, the volume for intracameral delivery is about 100 microliters, less than about 100 microliters, less than about 20 microliters, less than about 10 microliters, less than about 5 microliters, less than about 2.5 microliters, or about 1 microliter. Provided herein is an injectable formulation for intraocular delivery, comprising: a modified FGF-1 polypeptide comprising an amino acid sequence set forth in SEQ ID NO: 1, or having an amino acid sequence that is at least 90% identical to SEQ ID NO: 1, and comprising at least 1, 2, 3, 4 or 5 single amino acid mutations; and L-methionine. The modified FGF-1 polypeptide in the formulation may be present at greater than about 95% pure and the polypeptide is in monomeric form in the formulation. The formulation of claim 1, wherein the polypeptide further comprises an extension peptide positioned between the N-terminal methionine residue and the first residue of SEQ ID NO: 1. In some embodiments, the injectable formulation may comprise a modified FGF-1 comprising an amino acid sequence set forth in any one of the following sequences, SEQ ID NOs: 2, 205, 206, 3-8, 14-18, 24-28, 93-117, 118-141, 146-149, 174-204, 207, or a sequence that is at least 90% identical to the sequences, or is a fragment thereof.

In some embodiments, an injectable formulation is hereby provided, the formulation comprises a required dose and concentration of the modified FGF-1, and an excipient, comprising one or more of sodium chloride; ammonium sulfate; monobasic potassium phosphate; dibasic sodium phosphate dihydrate; ethylenediaminetetraacetic; and L-Methionine. In some embodiments, the injectable formulation may comprise: modified FGF1; at least about 50 mM dibasic sodium phosphate dihydrate; at least about 100 mM sodium chloride; at least about 10 mM ammonium sulfate; at least about 0.1 mM ethylenediaminetetraacetic acid (EDTA); at least about 5 mM L-Methionine; and at least about 0.01% polysorbate 80 (w/v). The injectable formulation comprising the modified FGF-1 polypeptide may comprise one or more mutations selected from the group consisting of: Cys16Ser, Ala66Cys, and Cys117Val, Lys12Val, Cys16Ser, Ala66Cys, Cys117Val, and Pro134Val, and wherein the modified FGF-1 polypeptide further comprises at least one residue of the peptide ALTEK. In some embodiments, the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1, and at least one residue of the peptide ALTEK located between the N-terminal methionine and position 1 of SEQ ID NO: 1.

In some embodiments, the formulation or the pharmaceutically suitable excipient therein comprises human serum albumin (HSA) and/or polysorbate 80. In some embodiments, the formulation comprises L-Methionine. In some embodiments, the L-Methionine is present at a concentration between 1 mM to 20 mM in the formulation. In some embodiments, the L-Methionine is present at a concentration between 2 mM to 10 mM in the formulation. In some embodiments, the L-Methionine is present at a concentration between 1 mM to 10 mM in the formulation. In some embodiments, the L-Methionine is present at a concentration between 2.5 mM to 15 mM in the formulation. In some embodiments, the L-Methionine is present at a concentration of about 5 mM in the formulation. For delivery of a composition comprising at least one of the modified FGF-1 polypeptides described herein via a mucosal route, which includes delivery to the nasal passages, throat, and airways, the composition may be delivered in the form of an aerosol. The compound may be in a liquid or powder form for intramucosal delivery. For example, the composition may be delivered via a pressurized aerosol container with a suitable propellant, such as a hydrocarbon propellant (e.g., propane, butane, isobutene). The composition may be delivered via a non-pressurized delivery system such as a nebulizer or atomizer.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, PA (2005)).

The modified FGF-1 polypeptides or pharmaceutical compositions described herein may be formulated for sustained or slow-release. Such compositions may generally be prepared using well known technology and administered by, for example, periocular, intraocular, rectal, oral or subcutaneous implantation, or by implantation at the desired target site, or by topical application. Sustained-release formulations may contain an agent dispersed in a carrier matrix and/or contained within a reservoir surrounded by a rate controlling membrane. Excipients for use within such formulations are biocompatible and may also be biodegradable; preferably the formulation provides a relatively constant level of active component release. The amount of active compound contained within a sustained-release formulation depends upon the site of implantation, the rate and expected duration of release, and the nature of the condition to be treated or prevented.

Systemic drug absorption of a drug or composition administered via an ocular route is known to those skilled in the art (see, e.g., Lee et al., Int. J. Pharm. 233:1-18 (2002)). In one embodiment, a compound described herein is delivered by a topical ocular delivery method (see, e.g., Curr. Drug Metab. 4:213-22 (2003)). The composition may be in the form of an eye drop, salve, or ointment or the like, such as, aqueous eye drops, aqueous ophthalmic suspensions, non-aqueous eye drops, and non-aqueous ophthalmic suspensions, gels, ophthalmic ointments, etc. For preparing a gel, for example, carboxyvinyl polymer, methyl cellulose, sodium alginate, hydroxypropyl cellulose, ethylene maleic anhydride polymer and the like can be used.

In another embodiment, the modified FGF solution or pharmaceutical composition (e.g., an ophthalmic formulation) contains hyaluronic acid, carboxymethyl cellulose, or other polysaccharides that provide increased ocular tolerability, viscosity and osmolality to produce a comfortable ocular solution.

The dose of the modified FGF or pharmaceutical composition comprising at least one of the modified FGF-1 polypeptides described herein may differ, depending upon the patient's (e.g., human) condition, that is, stage of the ocular disease, disorder, or condition, general health status, age, and other factors that a person skilled in the medical art will use to determine dose. When the composition is used as eye drops, for example, one to several drops per unit dose, preferably 1 or 2 drops (about 50 μl per 1 drop), may be applied about 1 to about 6 times daily.

Pharmaceutical compositions may be administered in a manner appropriate to the disease, disorder, or condition to be treated (or prevented) as determined by persons skilled in the medical arts. An appropriate dose and a suitable duration and frequency of administration will be determined by such factors as the condition of the patient, the type and severity of the patient's disease, disorder, or condition, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free, or a lessening of symptom severity). For prophylactic use, a dose should be sufficient to prevent, delay the onset of, or diminish the severity of an ocular disease, disorder, or condition. Optimal doses may generally be determined using experimental models and/or clinical trials. The optimal dose may depend upon the body mass, weight, or blood volume of the patient.

In various embodiments, a modified FGF-1 polypeptide of the present disclosure may be administered as a daily dose over a period of time to a subject. In some embodiments, a modified FGF-1 polypeptide of the present disclosure may be administered chronically or long-term. In some embodiments, a modified FGF-1 polypeptide of the present disclosure may be administered for a period of days, weeks, months, years or continued therapy over the lifetime of a subject. In some embodiments, a modified FGF-1 polypeptide of the present disclosure may be administered for a period of about 7 days, 15 days, about 21 days, about 30 days, about 3 months, about 6 months, about 12 months, about 18 months, about 2 years, about 5 years, about 7 years, about 10 years, about 15 years, about 20 years, about 25 years, about 30 years, about 35 years, or about 40 years. In some embodiments, a treatment regime may be determined for an individual subject dependent on various factors. In some examples, the treatment regimen is dependent on the level of exposure to a compound causing a chemical or thermal injury, such as a vesicant compound. In some embodiments, the treatment regimen is about 2 weeks for an acute exposure and several months to a year for a long-term exposure. In some embodiments, the treatment regimen is chronic. In some examples, a factor may include, but not be limited to, a determination of the change in the extent of degeneration of corneal tissue in response to administration of a modified FGF-1 polypeptide of the present disclosure. In some examples, a factor may include, but not be limited to, amelioration of MGK sequelae in response to administration of a modified FGF-1 polypeptide of the present disclosure. In some examples, a factor may include, but not be limited to, healing of corneal endothelial lesions in response to administration of a modified FGF-1 polypeptide of the present disclosure. In some examples, a factor may include, but not be limited to, corneal epithelial cell proliferation in response to administration of a modified FGF-1 polypeptide of the present disclosure. In some examples, a factor may include, but not be limited to, reduction of symptoms associated with Fuch's dystrophy in response to administration of a modified FGF-1 polypeptide of the present disclosure. In embodiments, a subject exhibiting an immediate response to the composition, for example, an immediate reduction in symptoms associated with Fuch's dystrophy, may require less frequent doses than a subject exhibiting a response to the composition at a later time or after several doses.

The doses of the modified FGF-1 polypeptides or pharmaceutical compositions can be suitably selected depending on the clinical status, condition and age of the subject, dosage form and the like. In the case of eye drops, a modified FGF described herein can be administered, for example, from about 10 ug/ml to about 100 mg/ml of the modified FGF one to seven times per week.

Also provided are methods of manufacturing the modified FGF-1 polypeptides and pharmaceutical compositions described herein. A composition comprising a pharmaceutically acceptable excipient or carrier and at least one of the modified FGF-1 polypeptides described herein may be prepared by synthesizing the modified FGF according to any one of the methods described herein or practiced in the art and then formulating the compound with a pharmaceutically acceptable carrier. Formulation of the composition will be appropriate and dependent on several factors, including but not limited to, the delivery route, dose, and stability of the compound.

At least one modified FGF described herein can be administered to human or other nonhuman vertebrates. In certain embodiments, the modified FGF is substantially pure, in that it contains less than about 5% or less than about 1%, or less than about 0.1%, of other organic molecules, such as contaminating intermediates or by-products that are created, for example, in one or more of the steps of a synthesis method. In other embodiments, a combination of one or more modified FGF-1 polypeptides described herein can be administered.

The compositions described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like.

In the case wherein the patient's condition does not improve, upon the doctor's discretion the administration of the compositions may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compositions may be given continuously; alternatively, the dose of drug being administered may be temporarily reduced or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more modified FGF-1 polypeptides. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers or resealable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Bulk Drug Substance Formulation and Pharmaceutical Formulation

An embodiment of the present disclosure provides a modified FGF-1 polypeptide (e.g., FGF-1 polypeptide comprising the sequence of SEQ ID NO: 2) as a bulk drug substance (BDS) in a formulation comprising at least one of sodium chloride, ammonium sulfate and di-sodium hydrogen phosphate dihydrate. In some embodiments, the BDS is stored as a solution in an eluate buffer (which is the formulation for storing the bulk drug substance), following purification from a host cell expressing the modified FGF-1 polypeptide using, for example, a Butyl Sepharose fast flow column. During the purification, the modified FGF-1 polypeptide typically elutes with a gradient and therefore, the eluate buffer, in some instances, has no precise composition. For instance, in some cases, the eluate buffer comprises sodium chloride, ammonium sulfate, and di-sodium hydrogen phosphate dihydrate and has a pH of about 7.4. In some embodiments, the bulk drug substance formulation comprises about 100 mM to about 1000 mM sodium chloride, such as about 200 mM, about 300 mM, about 400 mM, about 500 mM, about 600 mM, about 700 mM, about 800 mM, about 900 mM, about 1000 mM sodium chloride; about 100 mM to about 500 mM ammonium sulfate, such as about 200 mM, about 300 mM, about 310 mM, about 320 mM, about 330 mM, about 340 mM, about 360 mM, about 370 mM, about 380 mM, about 390 mM, about 400 mM, about 500 mM ammonium sulfate; and about 1 mM to about 50 mM di-sodium hydrogen phosphate dihydrate, such as about 2 mM, about 5 mM, about 10 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM di-sodium hydrogen phosphate dihydrate, or a combination thereof. In some embodiments, the BDS is stored in a formulation that has a pH from about 6 to about 8.

An embodiment of the present disclosure provides a pharmaceutical formulation composed of a histidine/polysorbate/sorbitol formulation at pH 5.8. Various advantages are associated with the pharmaceutical formulation include, but are not limited to, lack of visible particulates, the nearly constant main peak area by SE-HPLC, the low percentage of soluble aggregate over time by SE-HPLC, and the control of pH over time. In some embodiments, histidine is present at a concentration from about 0.1 mM to about 10 mM, such as about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 40 mM, about 50 mM, about 100 mM. In some embodiments, the pharmaceutical formulation comprises a surfactant. In some embodiments, the surfactant can include nonionic surfactants, anionic surfactants, cationic, amphoteric surfactants, and any combination thereof. In some embodiments, the surfactant is polysorbate. In some embodiments, the pharmaceutical formulation comprises a polysorbate, e.g., PS20, PS80. In some embodiments, the formulation comprises PS80 which is present at a concentration from about 0.01% to about 10%. In some embodiments, the formulation comprises polysorbate 80, NF (PS80) (% w/v) at a concentration of at least about 0.005%, at least about 0.01%, at least about 0.015%, at least about 0.02%, at least about 0.03%, at least about 0.04%, at least about 0.05%, at least about 0.06%, at least about 0.07%, at least about 0.08%, at least about 0.09%, or at least about 0.1%. In other embodiments, the formulation comprises from about 0.005% to about 0.1% PS80, from about 0.005% to about 0.02% PS80, from about 0.005% to about 0.05% PS80, from about 0.01% to about 0.02% PS80, from about 0.02% to about 0.1% PS80 or from about 0.01% to about 0.03% PS80. In still other embodiments, the composition comprises PS80 at a concentration of about 0.01%. In yet other embodiments, the composition comprises PS80 at a concentration of about 0.02%. In some embodiments, the composition comprises PS80 at a concentration of about 0.03%. In a particular embodiment, the composition comprises PS80 at a concentration of about 0.04%. In some embodiments, the composition comprises PS80 at a concentration of about 0.05%. In other embodiments, the composition comprises PS80 at a concentration of about 0.06%. In other embodiments, the composition comprises PS80 at a concentration of about 0.07%. In other embodiments, the composition comprises PS80 at a concentration of about 0.08%. In other embodiments, the composition comprises PS80 at a concentration of about 0.09%. In other embodiments, the composition comprises PS80 at a concentration of about 0.1%. In some embodiments, the formulation comprises polysorbate 20, NF (PS20) (% w/v) at a concentration of at least about 0.005%, at least about 0.01%, at least about 0.015%, at least about 0.02%, at least about 0.03%, at least about 0.04%, at least about 0.05%, at least about 0.06%, at least about 0.07%, at least about 0.08%, at least about 0.09%, or at least about 0.1%. In other embodiments, the formulation comprises from about 0.005% to about 0.1% PS20, from about 0.005% to about 0.02% PS20, from about 0.005% to about 0.05% PS20, from about 0.01% to about 0.02% PS20, from about 0.02% to about 0.1% PS20 or from about 0.01% to about 0.03% PS20. In still other embodiments, the composition comprises PS20 at a concentration of about 0.01%. In yet other embodiments, the composition comprises PS20 at a concentration of about 0.02%. In some embodiments, the composition comprises PS20 at a concentration of about 0.03%. In a particular embodiment, the composition comprises PS20 at a concentration of about 0.04%. In some embodiments, the composition comprises PS20 at a concentration of about 0.05%. In other embodiments, the composition comprises PS20 at a concentration of about 0.06%. In other embodiments, the composition comprises PS20 at a concentration of about 0.07%. In other embodiments, the composition comprises PS20 at a concentration of about 0.08%. In other embodiments, the composition comprises PS20 at a concentration of about 0.09%. In other embodiments, the composition comprises PS20 at a concentration of about 0.1%. In other embodiments, the pharmaceutical formulation is delivered to a subject by a suitable means, including, for example, intraocular injection, intracameral injection, periocular injection, intravenous injection, intraperitoneal injection, or intranasal injection.

In some embodiments, the pharmaceutical formulation comprises an alcohol compound. In some embodiments the alcohol compound is sorbitol, erythritol, xylitol, glycerol, mannitol, or any combination thereof. In some embodiments the alcohol compound is a tonicity modifying agent, e.g., a sorbitol. In some embodiments, the alcohol compound is sorbitol, which is present from about 1% to about 10%. In some embodiments, the formulation comprises a tonicity modifying agent which is sodium chloride. In some embodiments, the pharmaceutical formulation comprises glycerin.

In certain embodiments, the formulation comprises sorbitol (% w/v) USP at a concentration of at least about 0.25%, at least about 0.5%, at least about 0.75%, at least about 1%, at least about 1.5%, at least about 2%, at least about 2.5%, at least about 3%, at least about 3.5%, at least about 4%, at least about 4.5%, at least about 5%, at least about 7.5% or at least about 10%. In other embodiments, the composition comprises between about 0.5% and about 5% sorbitol, between about 0.5% and about 4% sorbitol, between about 0.5% and about 1.5% sorbitol, between about 1% and about 2% sorbitol, or between about 2.5% and about 3.5% sorbitol.

In some embodiments, the pharmaceutical formulation has a pH of about 5.8, from about 4.5 to about 6.5, from about 4.0 to about 6.0, from about 4.0 to less than about 6.5, from about 5.0 to about 6.0, from about 5.1 to about 5.9, from about 5.2 to about 5.8, from about 5.3 to about 5.4, from about 5.4 to about 5.5, from about 4.1 to about 6.1, from about 4.2 to about 6.2, from about 4.3 to about 6.3, from about 4.4 to about 6.4, from about 4.5 to about 6.5, from about 4.6 to about 6.6, from about 4.7 to about 6.7, from about 4.8 to about 6.8, from about 4.9 to about 6.9, from about 5.0 to about 7.0, from about 4.1 to about 5.1, from about 4.2 to about 5.2, from about 4.3 to about 5.3, from about 4.4 to about 5.4, from about 4.5 to about 5.5, from about 4.6 to about 5.6, from about 4.7 to about 5.7, from about 4.8 to about 5.8, from about 4.9 to about 5.9, from about 5.0 to about 6.0, less than about 6.1, less than about 6.2, less than about 6.3, less about 6.4, less than about 6.5, less than about 6.6, less than about 6.7, less than about 6.8, less than about 6.9, less than about 7.0.

In some embodiments, the modified FGF-1 polypeptide (e.g., modified FGF-1 polypeptide of SEQ ID NO: 2) comprises the active ingredient in a pharmaceutical formulation as described herein and is present at a concentration of from about 100 μg/mL to about 1000 μg/mL, from about 500 μg/mL to about 200 μg/mL, from about 0.0001 μg/mL to about 0.0005 μg/mL, from about 0.0005 μg/mL to about 1.0 μg/mL, from about 1.0 μg/mL to about 10 μg/mL, from about 10 μg/mL to about 20 μg/mL, from about 20 μg/mL to about 30 μg/mL, from about 30 μg/mL to about 40, from about 40 μg/mL to about 50 μg/mL, from about 50 μg/mL to about 60 μg/mL, from about 60 μg/mL to about 70 μg/mL, from about 70 μg/mL to about 80 μg/mL, from about 80 μg/mL to about 90 μg/mL, from about 90 μg/mL to about 100 μg/mL, from about 100 μg/mL to about 110 μg/mL, from about 110 μg/mL to about 120 μg/mL, from about 120 μg/mL to about 130 μg/mL, from about 130 μg/mL to about 140 μg/mL, from about 140 μg/mL to about 150 μg/mL, from about 150 μg/mL to about 160 μg/mL, from about 160 μg/mL to about 170 μg/mL, from about 170 μg/mL to about 180 μg/mL, from about 180 μg/mL to about 190 μg/mL, from about 190 μg/mL to about 200 μg/mL.

An embodiment of the present disclosure provides a bulk drug substance formulation comprises a modified FGF-1 polypeptide; sodium chloride at a concentration from at least about 200 mM to about 1000 mM; ammonium sulfate at a concentration of about 50 mM to about 500 mM; di-sodium hydrogen phosphate at a concentration of about 1 mM to about 50 mM. The modified FGF-1 polypeptide comprises an amino acid sequence that is at least 90% identical to SEQ ID NO:2 and comprises the following amino acid residues: Ser at position 16, Cys at position 66, and Val at position 117. In some embodiments, the concentration of the modified FGF-1 polypeptide is from at least about 0.1 g/mL to about 10 g/mL, such as from about 0.2 g/mL to about 15 g/mL, from about 0.3 g/mL to about 20 g/mL, from about 1 g/mL to about 5 g/mL, from about 1 g/mL to about 4 g/mL, from about 2 g/mL to about 3 g/mL. In some embodiments, the concentration of the modified FGF-1 polypeptide is about 3 g/mL. In some embodiments, the bulk drug substance formulation comprises sodium chloride at a concentration of about 800 mM. In some embodiments, the bulk drug substance formulation comprises ammonium sulfate at a concentration of about 320 mM. in some embodiments, the bulk drug substance formulation comprises di-sodium hydrogen phosphate at a concentration of about 20 mM. In some embodiments, the bulk drug substance formulation has a pH of from about 7 to about 9. In some embodiments, the bulk drug substance formulation has a pH of about 7.4. In some embodiments, the modified FGF-1 polypeptide is stable when stored at a temperature of −60° C.±10° C.

An embodiment of the present disclosure provides a method of manufacture that comprises purification of a refolded modified FGF-1 polypeptide isolated from inclusion bodies in a culture of bacterial cells transfected with a vector comprising a nucleic acid for encoding the modified FGF-1 polypeptide, wherein the purification comprises capturing of the refolded modified FGF-1 polypeptide using a chromatographic column packed with an agarose based resin (e.g., Capto™ DeVirS resin) followed by polishing by hydrophobic interaction chromatography, using a chromatographic column packed with Butyl Sepharose resin. In some embodiments, the recovery of the modified FGF-1 polypeptide from the polishing step is about 10% to about 40% greater, or about 10% to about 50%, 60%, 70%, 80%, 90%, or 100% greater, such as about 11% greater, about 12% greater, about 13% greater, about 14% greater, about 15% greater, about 16% greater, about 17% greater, about 18% greater, about 19% greater, about 20% greater, about 25% greater, about 30% greater, about 35% greater, about 40% greater, than recovery of the modified FGF-1 polypeptide after a polishing step by hydrophobic interaction chromatography, using a chromatographic column packed with Heparin resin, in a method of manufacture that is otherwise identical.

### Combination Treatments

The modified FGF-1 polypeptides and pharmaceutical compositions may also be used in combination with other therapeutic agents that are selected for their therapeutic value for the condition to be treated. The modified FGF-1 polypeptides and pharmaceutical compositions may also be used in combination with other therapeutic agents that are selected for their therapeutic value for treating the vesicant injury. Such agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the clinician. The initial administration can be made according to established protocols recognized in the field, and then, based upon the observed effects, the dosage, modes of administration and times of administration can be modified by the clinician.

The particular choice of these optional additional agents used will depend upon the diagnosis of the attending physicians and their judgment of the condition of the patient and the appropriate treatment protocol. The agents may be administered concurrently (e.g., simultaneously, essentially simultaneously or within the same treatment protocol) or sequentially, depending upon the nature of the disease, disorder, or condition, the condition of the patient, and the actual choice of agents used. The determination of the order of administration, and the number of repetitions of administration of each therapeutic agent during a treatment protocol, is well within the knowledge of the physician after evaluation of the disease being treated and the condition of the patient.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For example, the modified may be incorporated into formulations that contain other active ingredients such as steroids, antibiotics, anti-inflammatories, cytokines such as IL-1 or analogs of IL-1, or antagonists of cytokines such as inhibitors of IL-17.

Other exemplary cytokines include, but are not limited to, interleukins (e.g., IL-2, IL-3, IL-4, IL-5, IL-6, IL-7, IL-8, IL-9, IL-10, IL-11, IL-12, IL-13, IL-15, IL-16, IL-17, IL-18, IL-1α, IL-10, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and migration inhibitory factor MIF.

In some embodiments, combinations or pharmaceutical compositions described herein are administered in immunosuppressive therapy to reduce, inhibit, or prevent activity of the immune system. Immunosuppressive therapy is clinically used to: prevent the rejection of transplanted organs and tissues; treatment of autoimmune diseases or diseases that are most likely of autoimmune origin; and treatment of some other non-autoimmune inflammatory diseases.

In some embodiments, the modified FGF-1 polypeptides and pharmaceutical compositions described herein are administered with one or more anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, and COX-2 specific inhibitors (such as, but not limited to, celecoxib, rofecoxib, valdecoxib, parecoxib, etoricoxib, lumiracoxib, CS-502, JTE-522, L-745,337 and NS398).

Corticosteroids, include, but are not limited to: betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Other agents used as anti-inflammatories include those disclosed in U.S. patent publication 2005/0227929, herein incorporated by reference.

Some commercially available anti-inflammatories include, but are not limited to: Arthrotec® (diclofenac and misoprostol), Asacol® (5-aminosalicyclic acid), Salofalk® (5-aminosalicyclic acid), Auralgan® (antipyrine and benzocaine), Azulfidine® (sulfasalazine), Daypro® (oxaprozin), Lodine® (etodolac), Ponstan® (mefenamic acid), Solumedrol® (methylprednisolone), Bayer® (aspirin), Bufferin® (aspirin), Indocin® (indomethacin), Vioxx® (rofecoxib), Celebrex® (celecoxib), Bextra® (valdecoxib), Arcoxia® (etoricoxib), Prexige® (lumiracoxib), Advil®, Motrin® (ibuprofen), Voltaren® (diclofenac), Orudis® (ketoprofen), Mobic® (meloxicam), Relafen® (nabumetone), Aleve®, Naprosyn® (naproxen), Feldene® (piroxicam).

In one embodiment, compositions described herein are administered with leukotriene receptor antagonists including, but are not limited to, BAY u9773 (see EP 00791576; published 27 Aug. 1997), DUO-LT (Tsuji et al, Org. Biomol. Chem., 1, 3139-3141, 2003), zafirlukast (Accolate®), montelukast (Singulair®), prankulast (Onon®), and derivatives or analogs thereof.

In some embodiments, the modified FGF-1 polypeptides and pharmaceutical compositions described herein are administered with one or more Rho kinase inhibitors. In some embodiments, the modified FGF-1 polypeptides and pharmaceutical compositions described herein are administered with one or more additional growth factors, including, but not limited to epidermal growth factor (EGF) and nerve growth factor (NGF). See, e.g., see Joyce et al. (2009) Invest Ophthalmol. Vis Sci. 50:2116-2122, vascular endothelial growth factor (VEGF), transforming growth factor alpha and beta (TGF-alpha and TFG-beta), platelet-derived endothelial growth factor (PD-ECGF), platelet-derived growth factor (PDGF), tumor necrosis factor alpha (TNF-alpha), hepatocyte growth factor (HGF), insulin like growth factor (IGF), erythropoietin, colony stimulating factor (CSF), macrophage-CSF (M-CSF), granulocyte/macrophage CSF (GM-CSF) and nitric oxide synthase (NOS).

### Kits/Articles of Manufacture

For use in the therapeutic applications described herein, kits and articles of manufacture are also provided herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of ophthalmic formulations of the modified FGF-1 polypeptides and pharmaceutical compositions provided herein are contemplated as are a variety of treatments for any ocular disease, disorder, or condition that would benefit by administration of a modified FGF ore pharmaceutical composition described herein.

For example, the container(s) can include a modified FGF such as a modified FGF-1 having a sequence of SEQ ID NO: 2. The container(s) optionally have a sterile access port. Such kits optionally comprising compounds with an identifying descriptions or labels or instructions relating to their use in the methods described herein.

In some embodiments, a kit may be suitable for or designed to be suitable for an injectable liquid formulation for intraocular delivery. The kit may be designed as a low-volume vial and may comprise a conical insert. In some embodiments, the kit is the dropper bottle. In some embodiments, the dropper bottle may be enabled to provide at least on dose of modified FGF-1 in the injectable formulation. In some embodiments, the dropper bottle further comprises a sterile filter. In some embodiments, the container comprises the syringe. In some embodiments, the syringe comprises a material selected from the group consisting of tuberculin polypropylene and glass. In some embodiments, the syringe is prefilled with an injectable formulation. In some embodiments, the kit may further comprise an electronic control unit. In some embodiments, the electronic control unit enables control of administration of a volume of an injectable formulation according to that described in the preceding sections, wherein the volume is from at least about 10 microliters to about 100 microliters. In some embodiments, the kit of claim 107, wherein the dropper bottle is enabled to provide at least on dose of modified FGF-1 in the injectable formulation of any one of embodiments described above, or the pharmaceutical composition described anywhere in the disclosure. In some embodiments, the dropper bottle may further comprise a sterile filter. In some embodiments, the container comprises the syringe. In some embodiments, the syringe comprises a material selected from the group consisting of tuberculin polypropylene and glass. In some embodiments, the syringe is prefilled with an injectable formulation according to any one of embodiments described above, or the pharmaceutical composition described anywhere in the disclosure. The kit may further comprise an electronic control unit. In some embodiments, the electronic control unit enables control of administration of a volume of an injectable formulation according to any one of claims 1-45 or, a pharmaceutical composition according to any one of claims 90-95, wherein the volume is from at least about 10 μL to about 100 μL.

In some embodiments, a kit may typically include one or more additional containers, each with one or more of various materials (such as reagents, optionally in concentrated form, and/or devices) desirable from a commercial and user standpoint for use of a modified FGF described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a modified FGF pharmaceutical composition can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a modified FGF provided herein formulated in a compatible pharmaceutical carrier can also be prepared, placed in an appropriate container, and labeled for treatment of an indicated condition.

### Examples

These examples are provided for illustrative purposes only and not to limit the scope of the claims provided herein. The starting materials and reagents used in the examples described herein may be synthesized or can be obtained from commercial sources.

Example 1: Exemplary methods for evaluating therapeutic effects of modified FGF-1 polypeptides: Modified FGF-1 polypeptides comprising the sequence of SEQ ID NO: 2 (N-Met-TTHX1114) or SEQ ID NO: 205 (TTHX1001), generated using methods as described in the Recombinant Techniques section were evaluated for biological activity using a NIH-3T3 fibroblast cell proliferation assay. Modified FGF-1 polypeptides of SEQ ID NO: 2 and SEQ ID NO: 205 both presented comparable capabilities in terms of effectivity in inducing proliferation of the fibroblast cells. Modified FGF-1 polypeptides comprising the sequence of SEQ ID NO: 2 (N-Met-TTHX1114) or SEQ ID NO: 205 (TTHX1001) are further evaluated in experimental models of ophthalmic diseases, as described below.

Primary cultures (passage 1) of human corneal endothelial cells from a healthy donor are seeded onto 24 well plates in the presence of fetal bovine serum (FBS, 8%) and 24 hours later treated with the varying concentrations of N-Met-TTHX1114 (SEQ ID NO: 2), TTHX1001 (SEQ ID NO: 205), or wt-FGF-1 (SEQ ID NO: 1) in media with low (0.8%) FBS. The 8% FBS group serves as positive control. Results indicate that N-Met-TTHX1114 is more potent than TTHX1001 or wt-FGF-1 in stimulating human corneal epithelial cell proliferation and is dose responsive therein. The EC₅₀ of N-Met-TTHX1114 is about 100-fold lower than the wt-FGF-1 or the other tested modified FGF-1 polypeptide (TTHX1001; SEQ ID NO: 205).

An exemplary corneal injury model using nitrogen mustard (NM) can be used for evaluating the therapeutic effect of the pharmaceutical compositions described herein. A rabbit corneal organ culture model system was used to evaluate healing after exposure to NM. Rabbit eyes (8-12 weeks old) are obtained and corneas with 2-mm scleral rims are dissected from the eyes, placed epithelial-side down into a spot plate, and the concavities were filled with 558C molten agar (0.75%) in Dulbecco's modified Eagle's medium (DMEM). Once the solution is gelled, the corneas are inverted so that the epithelial layer is accessible. Cultures are placed in 60-mm diameter pyrex tissue culture dishes. High glucose DMEM is prepared containing 13MEM-NEAA (minimal essential medium non-essential amino acids), 13 RMPI 1640 Vitamin Solution, 13 antibiotic/antimycotic, ascorbic acid (0.45 mM), and ciprofloxacin (10 lg/ml). High glucose DMEM is added up to the scleral rims, leaving the corneas exposed to air. The dishes are placed in a 37° C. humidified incubator with 5% CO₂. The epithelium of each culture is moistened with 500 μL medium, added dropwise onto the central cornea every 7 to 9 hours. The vesicating agent, NM, is added dropwise onto the central cornea. Cornea samples (peeled off their agar support) are either put epithelial side down in cryomolds containing Optimal Cutting Temperature (OCT, Tissue-Tek; Sakura, Torrance, CA, USA) compound and flash frozen for histology and immunofluorescence, or directly snap frozen for further protein analyses including Western blot and ADAM17 activity assays (InnoZyme TACE activity assay kit; Calbiochem, Billerica, MA, USA).

After applying NM onto the central corneas, the cultures are returned to the 37° C. incubator for 2 hours without removing the vesicant. After this incubation, contaminated medium is removed, and fresh medium is added to the central cornea until the level in the dish reached the top of the scleral rim. Control unexposed and exposed corneas are then returned to 37° C. for a 22-hour incubation, being removed for only three short periods to add 20 μL medium to the exposed samples not receiving N-Met-TTHX1114 therapy, or to add 20 μL of N-Met-TTHX1114 as therapy to the central corneas. The first -met-TTHX1114 application is left on for 8 hours, the second for 9 hours, and the third for 5 hours. Thus, the length of the 2-hour exposure and the subsequent treatment is 24 hours in total.

Injury inflicted by NM includes the following: (a) hyperplasia of the epithelial layer, which is apparent by the increase in the number and depth of epithelial cells pushing down into the stroma. This is referred to as downward hyperplasia. Unexposed (naïve) cornea shows some downward hyperplasia but it isn't as extensive as cornea exposed to NM; (b) basal cell nuclei rising up toward the top of the basal epithelial cells; and (c) epithelial-stromal separation. The histopathological effects are visible as early as four days post-exposure. Histopathological grading is improved by treatment with N-Met-TTHX1114. The N-Met-TTHX1114 treated corneal sections exhibit lower score (indicative of lesser injury) compared to sections from untreated corneas. Peripheral corneal epithelial layer stimulation is assessed by via EdU incorporation of corneal epithelial cells (CECs). Primary cultures of rabbit CECs are established using standard procedures, e.g., the procedure described by Kay et al. (Kay et al. Investigative ophthalmology & visual science. 1993; 34(3):663-72; Lee et al., Investigative ophthalmology & visual science. 2009; 50(5):2067-76). The cells are exposed to NM for two hours. Proliferation assays are performed in 12-well plates using, e.g., a Click-IT assay kit (Life Technologies). Incorporation of EdU into corneal epithelial cells is an indicator of epithelial proliferation. The percentage corneal epithelial cells incorporating EdU are lower when treated with N-Met-TTHX1114, following NM-exposure, compared to untreated controls.

In a similar model, sulfur mustard can be used to induce experimental corneal endothelial injury and N-Met-TTHX1114 (or sham) can be administered for testing resolution. Eight weeks after exposure to rabbit cornea, endothelial cell morphology and structure are compared between test group (also referred to as resolved eyes) and sham control group (which later develops MGK). Resolved eyes are distinguished by the absence of characteristic MGK sequelae during clinical evaluations such as corneal erosions, neovascularization, or corneal haze and had corneal thicknesses that are statistically indistinguishable from sham-exposed controls by 6 weeks. Enface scanning micrographs of resolved eyes are found to be strikingly similar to sham-exposed controls, with a well-organized monolayer of polygonal cells. The average CEC size is increased in resolved eyes compared with control corneas; otherwise, resolved corneas do not exhibit significant variability across the posterior surface. In contrast, the sham-control treated rabbits with MGK endothelia reveal extensive variability in cell shape and cell size among animals, indicative of a dynamic injury process. Focal variability in endothelial morphology is routinely observed in individual corneas, with some regions exhibiting enlarged but mosaic CECs and other regions displaying significant disorganization, with variable degrees of apical blebbing, areas showing denuded DM, and clearly delineated cell boundaries lacking. These phenomena are not observed in the N-Met-TTHX1114 treated resolved endothelium. Transmission Electron Microscope images of N-Met-TTHX1114 treated resolved corneas are very similar to naïve endothelium. In contrast, sham-control treated endothelium with MGK pathology exhibit diffusive thickening of the posterior DM, consistent with either edema and/or the deposition of a retrocorneal fibrous membrane. The MGK corneas also exhibit extensive markers of CEC stress or injury, including cytoplasmic rarefication, excessive vacuolization, and swollen endoplasmic reticuli. There is a high frequency of overlapping cell processes, similar to 24-hour images and suggestive of an ongoing attempt to repopulate recently denuded DM.

The following methods can be adopted to evaluate the therapeutic effect of FGF-1 polypeptides e.g., modified FGF-1 polypeptide comprising the sequence of SEQ ID NO: 2 (N-Met-TTHX1114) or SEQ ID NO: 205 (TTHX1001) for the treatment of herpetic keratopathy.

A group of patients with herpetic keratopathy is selected for this study. The patients are divided into three subgroups. Patients in the first sub-group are administered, ocularly, an ophthalmic formulation, such as an eye drop, containing about 500 μg/ml (i.e., 0.0005 μg/ml) of N-met TTHX1114 (SEQ ID NO: 2) formulated in phosphate buffered saline (at pH 7.2), 0.3% propylene glycol, 0.4% polyethylene glycol 400, and 0.05% hydroxypropyl guar. Patients in the second sub-group are administered, ocularly, an ophthalmic formulation, such as an eye drop, containing about 500 μg/ml (i.e., 0.0005 μg/ml) of TTHX1001 (SEQ ID NO: 205) formulated in phosphate buffered saline (at pH 7.2), 0.3% propylene glycol, 0.4% polyethylene glycol 400, and 0.05% hydroxypropyl guar. Patients in the third sub-group are administered, ocularly, a sham ophthalmic formulation that does not contain the N-Met-TTHX1114 (SEQ ID NO: 2) or the TTHX1001 (SEQ ID NO: 205) but is otherwise identical to what is administered to the first and the second sub-group. For all three subgroups, the eye drop is either self-administered by the patient or administered by a nurse or a caregiver. The N-Met-TTHX1114 (SEQ ID NO: 2) containing ophthalmic formulation, the TTHX1001 (SEQ ID NO: 205) containing ophthalmic formulation, and the sham ophthalmic formulation are administered, respectively to patients in the first, second, and the third sub-group, twice daily for up to 30 days.

It is observed that the ophthalmic formulation containing the N-Met-TTHX1114 (SEQ ID NO: 2) and the ophthalmic formulation containing the TTHX1001 (SEQ ID NO: 205) results in healing of the herpetic corneal ulcer within about 14 days in majority of the patients belonging to the first and the second sub-groups, along with reduction in the duration of pain and inflammation. Furthermore, eyes of patients in the first and the second sub-groups, treated respectively with the N-Met-TTHX1114 (SEQ ID NO: 2) containing ophthalmic formulation and the TTHX1001 (SEQ ID NO: 205) containing ophthalmic formulation have less corneal haze and scarring than patients in the third sub-group, who were treated with the sham.

In order to study the effects of modified FGF-1 polypeptides comprising, for example, the sequence of SEQ ID NO: 206 (TTHX1114) or SEQ ID NO: 205 (TTHX1001) on Human Corneal Endothelial Cell (HCEC) proliferation, primary cultures (passage 1) of human corneal endothelial cells from a healthy donor are seeded onto 24 well plates in the presence of fetal bovine serum (FBS, 8%) and 24 hours later treated with the varying concentrations of TTHX1114 (SEQ ID NO: 206), TTHX1001 (SEQ ID NO: 205), or wt-FGF-1 (SEQ ID NO: 1) in media with low (0.8%) FBS. The 8% FBS group serves as positive control. Results indicate that TTHX1114 was more potent than TTHX1001 or wt-FGF-1 in stimulating human corneal epithelial cell proliferation and was dose responsive therein. The EC₅₀ of TTHX1114 was about 100-fold lower than the wt-FGF-1 or the other tested modified FGF-1 polypeptide (TTHX1001; SEQ ID NO: 205).

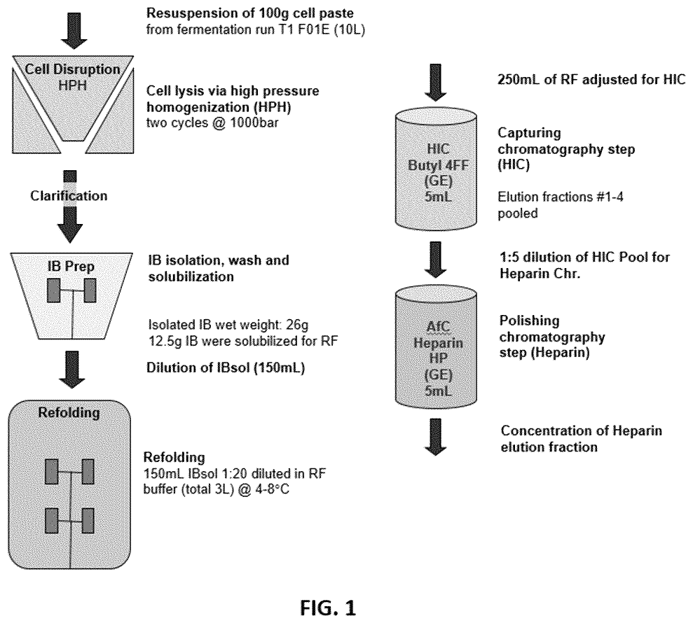

Example 2: Method of making scalable recombinant human FGF-1: An exemplary generalized method for producing an FGF-1 as an intraocular therapeutic is depicted in the schematic in FIG. 1. The generalized method is further developed and various modifications are made. Briefly, E. coli are transformed with a plasmid comprising the recombinant nucleic acid sequence encoding a modified human FGF-1 (N-Met-TTHX1114).

In one exemplary set, an E. coli codon optimized human FGF-1 (TTHX1114) sequence was subcloned in pET26b(+) vector using the Nde1/BamH1 cloning restriction sites to obtain pET26_TTHX1114 plasmid. An exemplary E. coli codon optimized sequence encoding hFGF-1 is provided below:

CATATGTTTAATCTGCCGCCGGGTAACTATAAGAAACCGAAACTGTTGTA
CAGCTCTAATGGTGGCCACTTCCTGCGTATCCTGCCGGACGGCACCGTCG
ATGGTACCCGTGACCGCAGCGATCAACACATTCAACTGCAACTGAGCGCC
GAGAGCGTGGGCGAAGTTTACATTAAGTCCACTGAAACGGGCCAGTACCT
GTGTATGGACACCGATGGCCTGCTGTACGGTTCGCAGACGCCAAATGAAG
AGTGCCTGTTCTTGGAGCGTCTGGAAGAGAACCACTATAACACCTACATT
AGCAAGAAACATGCGGAGAAAAACTGGTTTGTGGGTCTGAAGAAAAATGG
TTCCGTCAAGCGCGGTCCTCGTACGCATTATGGCCAGAAAGCAATCTTGT
TCCTGCCGCTGCCGGTTAGCAGCGACTAATGACTCGAG.

In one exemplary set, an E. coli codon optimized human FGF-1 sequence was subcloned using Nde1/BamH1 sites in a pMKet vector backbone to obtain pMKet_TTHX1114 plasmid, without a leader sequence, such as without an ompA leader sequence. The pMKet vector includes a pBR322 on sequence.

In exemplary one set, an ompA leader sequence is inserted upstream of the TTHX1114 coding sequence to obtain an OA_TTH1114pMKet plasmid that directs periplasmic expression of the FGF-1 polypeptide. An exemplary nucleic acid sequence encoding a leader ompA sequence is provided below:

5'-ATGAAAAAGACAGCTATCGCGATTGCAGTGGCACTGGCTGGTTTCGC
TACCGTTGCGCAAGCT-3'.

An exemplary ompA amino acid sequence is MKKTAIAIAVALAGFATVAQA.

The FGF-1 sequence is tested with or without the N terminal fMet.

Host cells tested are E. coli BL 21 strain, and E. coli W3110 strain.

Host cells are grown in test media of 2YT, or custom-made synthetic media comprising glucose or glycerol as carbon source. The cultures were left in presence of kanamycin throughout.

The transformed E. coli cells were expanded in a fermentation culture—and a 10 L aliquot was used for the exemplary FGF-1 production. Resuspension of 100 g E. coli cell paste from fermentation run T1 F01E (10 L) was followed by cell lysis, isolation of inclusion bodies (IB), -dilution in solubilizing buffer, refolding in refolding buffer (1:20 vol/vol). 250 ml of the refolding buffer comprising the FGF-1 was passed into butyl and heparin HIC columns.

In one exemplary run using the generalized protocol described above, the yield about 0.6 mg FGF-1 per L of refolding buffer. Approx. 0.2-0.25 mg of FGF obtained from 0.25 L refolding (around 1 mg/L). Identity of purified protein was confirmed by Western Blot.

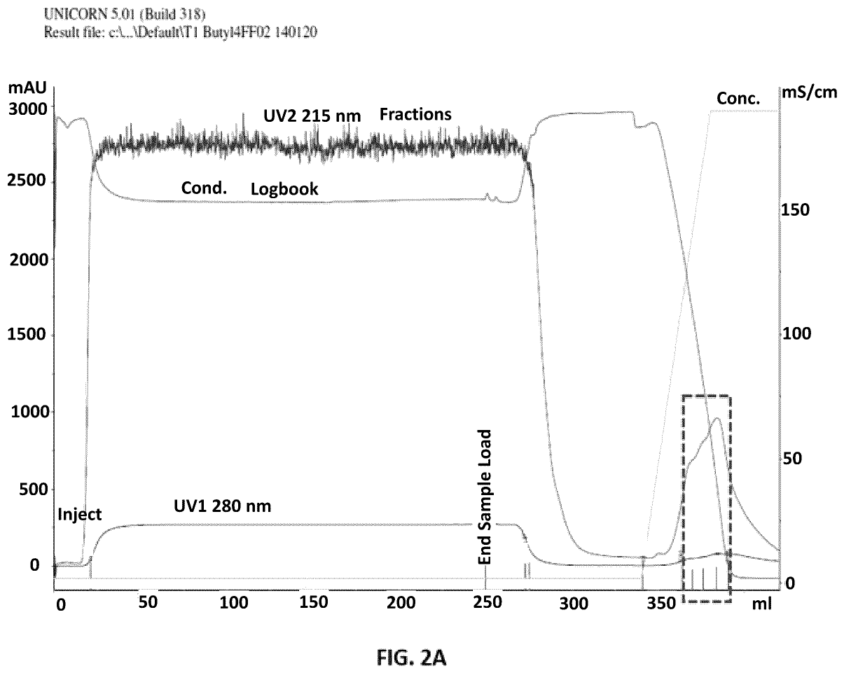

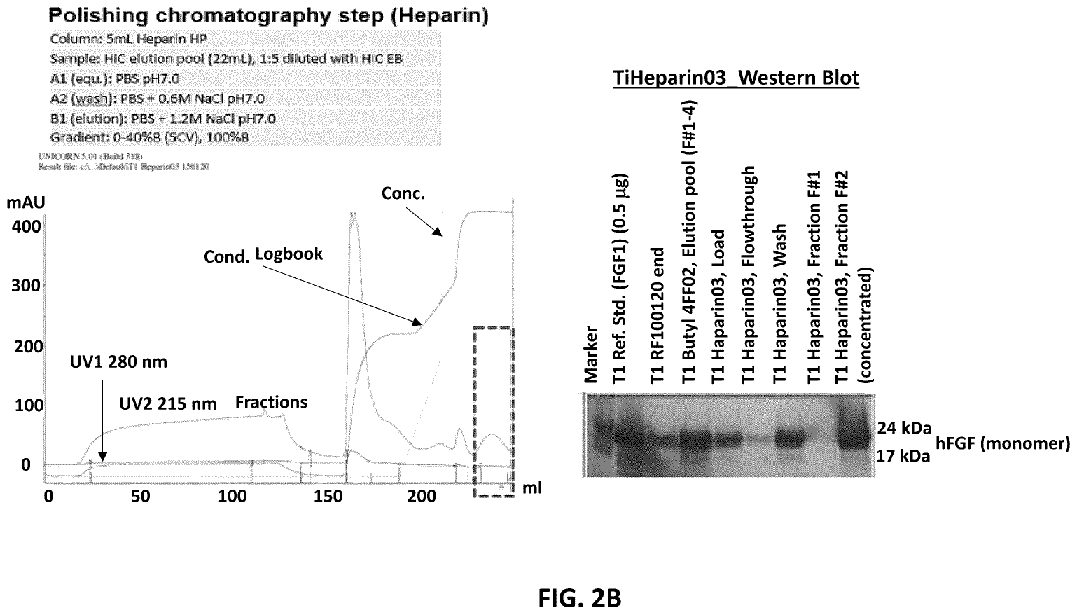

In an exemplary run, using pMKet TTX1114 (without the ompA leader sequence) in BL21 cells, and growing in a synthetic media resulted in an improved yield over a plasmid comprising an ompA leader sequence, improved the yield by about 10-fold. FIG. 2A shows eluent from Butyl capture chromatography column (e.g., Capto DeVirs (Cytiva, BPG 300×500, Part #17-5466)) and FIG. 2B shows eluent from heparin HIC, detected at A₂₁₅ and A₂₈₀ as indicated. The eluents fractions indicated by dotted lines were run on SDS-PAGE and western blot analysis showing presence of a monomer. The column used in this assay was a 5 ml Butyl 4FF, sample load 250 ml adjusted by addition of 1.5 M ammonium sulfate; eluted with 10 mM Na₂HPO₄, 2 mM KH₂PO₄ at pH 7.4. Exemplary western blot result from the Heparin column eluted sample is shown in FIG. 2B. In another exemplary method, several adjustments were made in the protocol. A clone screen for 1 L culture was run using conditions as indicated in the Table 1 below:

TABLE 1
Screening for clones in 1 L culture

| Fermentation ID | 1 | 2 | 3 | 4 |
|---|---|---|---|---|
| Cell line | BL21/TTHX1114_pMKet | | | BL21/OA_TTHX1114_pMKet |
| Medium | 2YT | Synthetic media | Synthetic media | Synthetic media |
| | | MMI_{Gly} + 16 g/L | MMI_{Gly} | MMI_{Gly} |
| | | Pepton + 10 g/L | | |
| | | Yeast | | |
| Temperature (Induction) | 37° C. | | | 20° C. |
| Antibiotic (kanamycin) | Yes | Yes | Yes | Yes |
| pO₂ | 30% | | | |
| pH | 6.8 | | | |
| Start carbon source | 30 g/L glycerol | | | |
| (batch) | | | | |
| Induction | 1 mM IPTG | | | |
| Induction length | 20 hours | | | |
| Final OD/ | 70/55 gL⁻¹ | 250/200 gL⁻¹ | 136/220 gL⁻¹ | 92/160 gL⁻¹ |
| cell paste [g/L] | | | | |
| Productivity PoI [g/L])¹ | 0.6 | 3.0 | 0.1 | 0.3 |

From the above optimization study, a high density of cells was obtained. Synthetic media and E. coli BL21 strain transformed with a pMVKet plasmid having a sequence encoding the codon optimized FGF-1 led to an unexpected high yield of 3 g/L FGF-1, which is an increase of about 5-30 fold over other experimental variations, as shown in the table.

In an exemplary method for making the modified FGF-1, the steps performed were as follows:
10 g of prepared IB solubilized in 50 mM Tris, 2 mM EDTA, 6 M Guanidine, 0.1 M NaCl, pH7.4
Reduction of hFGF by addition of 50 mM DTT (over 2 h)
Removal of DTT via diafiltration/ultrafiltration
Refolding via fast dilution to A280 of approx. 1.0 (RF overnight)
Refolding buffers:
   I (RFB): 1 M Arginine, 50 mM Tris, 0.1 M NaCl, 5 mM EDTA, pH 9.5
   II (RFD): 5 mM Tris, 2 mM Cystine, 5 mM Cysteine, pH 9.0
Centrifugation in order to remove precipitants
For capturing via Heparin: pH adjustment and dilution to reduce cond. <25 mS/cm (if needed), followed by salt adjustment of Heparin eluate by HIC (C4)
Column: Heparin HiTrap HP (5 mL)
   Buffers:
      I (equ./wash I): PBS pH7.0
      II (wash II): PBS+0.6 M NaCl pH7.0
      III (elution): PBS+1.2 M NaCl pH7.0
   Gradient: 0-100%0 buffer III (IOCV)
   Flow rate: 5 mL/min
For capturing via HIC (C4): salt addition to 1.5 M Ammonium Sulfate, pH adjustment to pH7.4.

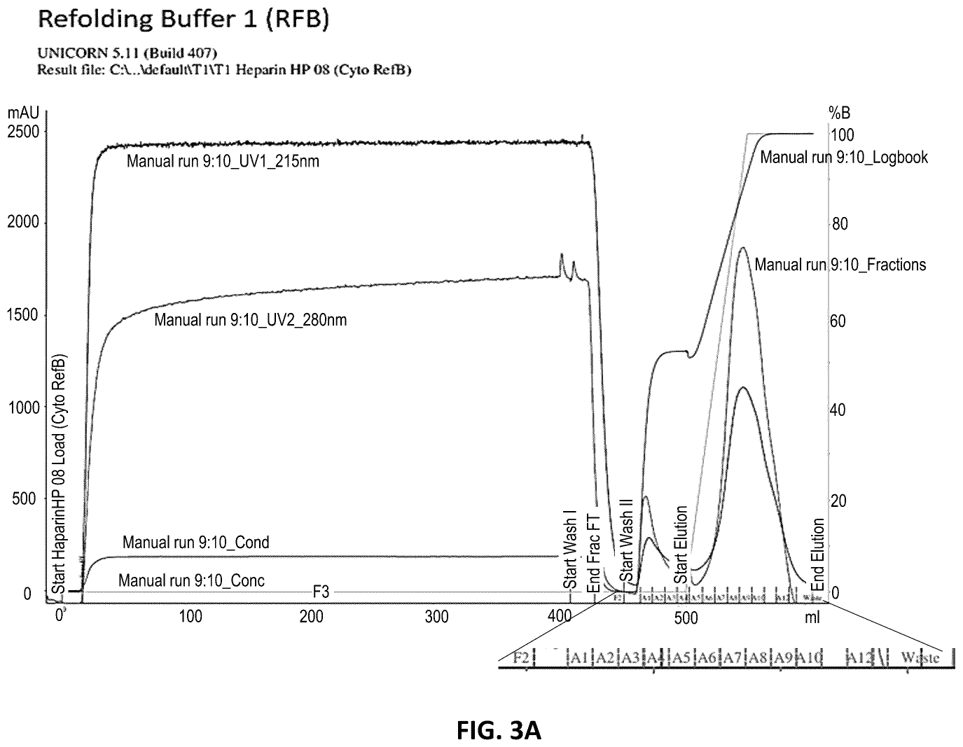

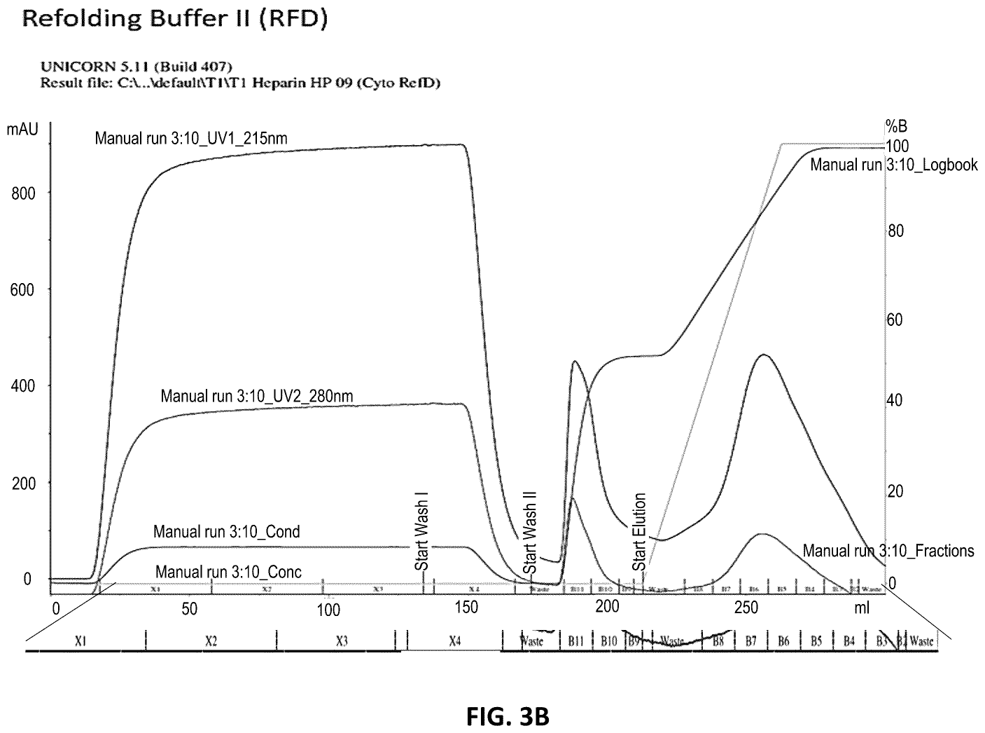

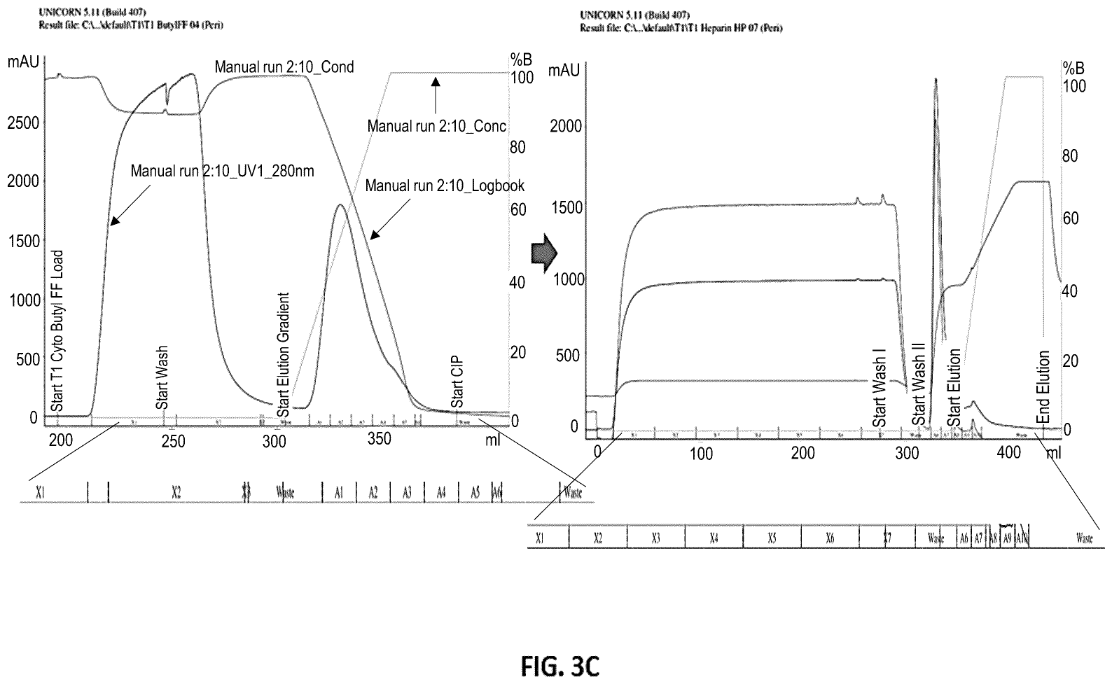

The respective eluent profiles from using Refolding buffers I and II are shown in FIGS. 3A and 3B. Corresponding SDS-PAGE images after Heparin HiTrap were evaluated (not shown). These studies indicated that using Refolding Buffer 1 (RFB) yielded significantly better results over using Refolding Buffer 2 (RFD). RFD resulted in heavy precipitates.

In another exemplary study, capture screening using HIC was performed in ambient temperatures. The following procedure was used:
10 g of cell pellet resuspended in 12 mM PO₄, pH7.0
Cell lysis via HPH (2×400 bar, T<25° C.)
Centrifugation @ 17500×g, 30 min, 8° C.
For capturing via Heparin: 50 mL lysate→pH adjustment→load
For capturing via HIC (C4): 50 mL lysate→salt addition to 1.5 M Ammonium Sulfate, pH adjustment to pH7.4→load.

Spectrometric data is shown from eluted butyl HIC, FIG. 3C (left), which was thereafter run in heparin column, yielding sharper product peak FIG. 3C (right).

In another exemplary study, a comparison was made for solubilization in Urea and guanidine buffers. Solubilization of inclusion bodies (IBs) in urea and guanidine with or without washing the IBs was tested. Also tested was the use of polysorbate 20 or 80 in washing the IBs. Results indicated that both PS20 and PS80 had comparable effect. Purity of solubilized FGF was comparable between both wash approaches. Yield of solubilized FGF was comparable for both detergents.

In an exemplary method, the clone is further optimized for microbial expression. New constructs were transformed into different strains and grown and FGF productivity was tested in different media. Each construct/strain combination was grown in custom synthetic medium, or in 2TY medium. The carbon source was either glycerol or glucose. After growing to OD₆₀₀ of 2, the culture was induced with 1 mM IPTG (c: expression @ 37° C. for 20 h; p: expression @ 26° C. for 20 h). Periplasmatic constructs were conducted with Osmotic Shock and afterwards treated with ultrasonic sound to break open the cells. Constructs for periplasmic expression and cytoplasmic expression are generated as follows (Table 2 and Table 3):

TABLE 2
Constructs for periplasmic expression

| Promotor | ori | Leader sequence | Strain |
|---|---|---|---|
| tac | pBR322 | ompA | BL21 |
| tac | pBR322 | ompA | W3110 |

TABLE 3
Constructs for cytoplasmic expression

| Promotor | ori | Leader sequence | Strain |
|---|---|---|---|
| T7 | pBR322 | — | BL21(DE3) |
| tac | pBR322 | — | BL21 |

Comparison of cytoplasmic constructs expressed in BL21 strain and W3110 strain and periplasmic constructs expressed in BL21 strain and W3110 strain were performed. In addition, all constructs were cultivated in in-house and 2TY media with glycerol as well as glucose as Carbon source. After growing to OD₆₀₀ of 2, the culture was induced with 1 mM IPTG (cytoplasmic: expression @ 37° C. for 20 h; periplasmic: expression @ 26° C. for 20 h). Periplasmatic constructs were conducted with Osmotic Shock and afterwards treated with ultrasonic sound to break open the cells.

pMKet constructs were significantly better for microbial expression of the modified FGF-1 protein over existing plasmids without the promoter, or other modifications described here.

In another exemplary study, urea was used in refolding buffer, and PS 20 was used as detergent. Guanidine was used for denaturation. Refolding was performed in RT and 2-8° C. (refolding buffers as disclosed previously). After the refolding, samples were diafiltrated using spin columns to eliminate DTT. Each RF approach was diluted due to high conductivity. Heparin column was set up with 5 ml HiTrap Heparin, using Buffer A (equilibration): PBS, pH 7.0; Buffer B (wash): PBS, 0.6 M NaCl, pH 7.0; Buffer C (elution): PBS, 1.2 M NaCl, pH 7.0. Samples were taken after each step for analysis using SDS-PAGE. The set-up is summarized in Table 4 below:

TABLE 4
Refolding conditions

| Heparin# | RF | RF temp | Dilution factor prior to load |
|---|---|---|---|
| 08A | A | 4° C. | 6 |
| 08B | B | 4° C. | 6 |
| 08C | C | 4° C. | 24 |
| 08D | D | 4° C. | 24 |
| 09A | C | 4° C. | 6 |
| 09B | D | 4° C. | 6 |
| 10A | A | RT | 6 |
| 10B | B | RT | 6 |
| 10C | C | RT | 6 |
| 10D | D | RT | 6 |

Table 5 shows the respective yields:

TABLE 5
Summary of yields

| FRACTION NAME | mg |
|---|---|
| Standard 1:10 (0.2 ug) | |
| Standard 1:20 (0.1 ug) | |
| Hep 8A wash | 0.1 |
| Hep 8A elu | 2.2 |
| Hep 8B wash | 0.0 |
| Hep 8B elu | 3.5 |
| Hep 8C wash | 0.2 |
| Hep 8C elu | 0.2 |
| Hep 8D wash | 0.0 |
| Hep 8D elu | 0.1 |
| Hep 9Awash | 0.0 |
| Hep 9A elu | 0.0 |
| Hep 9B wash | 0.0 |
| Hep 9B elu | 0.0 |
| Hep 10 Awash | 0.0 |
| Hep 10A elu | 1.0 |
| Hep 10B wash | 0.1 |
| Hep 10B elu | 1.4 |
| Hep 10C wash | 0.0 |
| Hep 10C elu | 0.0 |
| Hep 10D wash | 0.0 |
| Hep 10D elu | 0.2 |

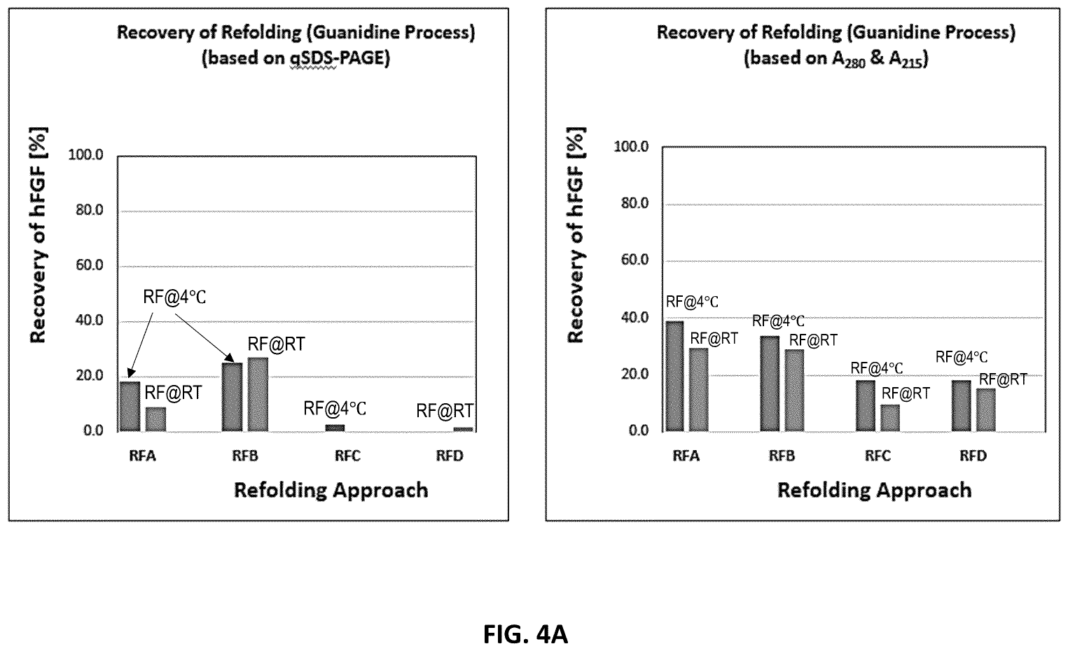

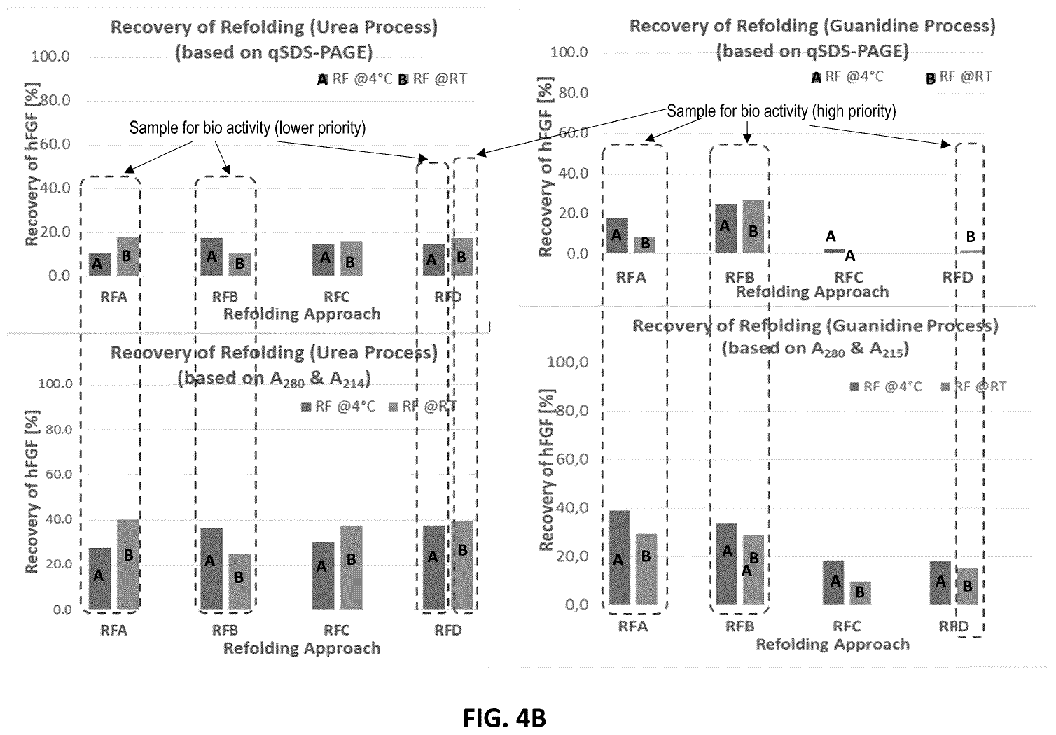

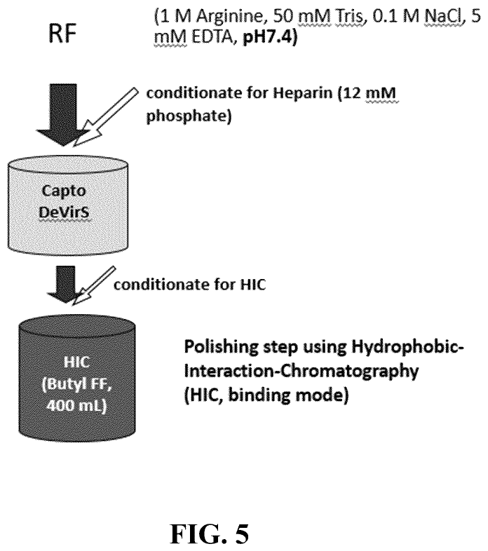

The proteins are resolved in SDS-PAGE, and the quantitated FGF-1 recoveries are shown in data represented in FIGS. 4A-4B. Size exclusion chromatography was set up using Agilent Advance Bio SEC 300A, pore size 2.7 μm. The running buffer included PBS with 0.5 M NaCl at pH 7.4. Flow rate was adjusted to 0.75 mL/min, and was run for 20 minutes. Absorbance was observed at A215 nm. The respective SEC peaks were evaluated for each run. Refolding buffer A and B showed better results in the process involving Guanidine as denaturing solution. Also, it appears that chaotropic agent used for IB solubilization did not affect performance of refolding. Refolding buffer C and D are slightly better than A and B (lower costs) when urea was used. It was also seen that the refolding was slightly better at 4° C.

### Example 3: Manufacturing Conditions of Bulk Drug Substance

### Fermentation and Primary Recovery of Inclusion Bodies

An exemplary study involves the fermentation, harvest/recovery of inclusion bodies, purification, isolating and testing of modified FGF-1 polypeptide (SEQ ID NO: 2) Bulk Drug Substance (BDS). The cGMP manufacturing of TTHX1114 BDS was performed using a master cell batch (MCB) of E. Coli BL21 pMKet comprising the nucleic acid sequence for expression of the modified FGF-1 polypeptide of SEQ ID NO: 2.

The content of a thawed vial containing the MCB was mixed and used to inoculate seed flasks containing synthetic medium for pre-culture: 5.2 g/L (NH₄)₂SO₄, 4.4 g/L NaH₂PO₄×2 H₂O, 4.0 g/L KCl, 5.3 g/L Citric×H₂O, 1.3 g/L Na₂HPO₄×2 H₂O, 0.5 g/L NaCl, 1 g/L MgSO₄×7 H₂O, 0.3 g/L CaCl₂)×2H₂O, 0.1 g/L FeCl₃×6 H₂O, 1×TES (21 mg/L ZnSO₄×7H₂O, 24 mg/L MnSO₄×H₂O, 8 mg/L CuSO₄×5 H₂O, 4 mg/L CoSO₄×7H₂O, 0.3 mg/L H₃BO₃, 0.2 mg/L Na₂MoO₄×2 H₂O), glycerol 10 g/L, kanamycin at 50 mg/L, 10 g/L bacto yeast extract and 16 g/L phytone peptone. The inoculated seed flasks were incubated at 37° C. until the culture reached an OD of about 2-5.

The main culture was carried out with a starting volume of 50 L of culture medium (30 g/L glycerol, 5.2 g/L (NH4)2SO4; 4.4 g/L NaH2PO4×2H2O; 4.0 g/L KCl; 5.3 g/L Citric acid×H2O; 1.3 g/L Na2HPO4×2 H2O; 0.5 g/L NaCl; 1.0 g/L MgSO4×7H2O; 0.3 g/L CaCl2)×2H2O; 0.1 g/L FeCl3×6 H2O; 1×TES (21 mg/L ZnSO4×7H2O, 24 mg/L MnSO4×H2O, 8 mg/L CuSO4×5 H2O, 4 mg/L CoSO4×7H2O, 0.3 mg/L H3BO3, 0.2 mg/L Na2MoO4×2 H2O), 1 mL/L antifoam, 10 g/L bacto yeast extract and 16 g/L phytone peptone in a 150 L bioreactor. The main culture was inoculated with 500 mL of pre-culture and was then incubated at 37° C. at pH 6.8 without oxygen limitation. The pH was regulated by addition of 25% ammonium hydroxide and 1 M phosphoric acid. After the initial amount of glycerol in the culture medium (30 g/L) was consumed, which is indicated by a pO2-peak, a constant feed with 45% aq glycerol at a feed rate of 2.333 kg/h was started. Approximately 10 hours after the start of feeding, product formation was induced by addition of isopropyl-β-D-1-thio-galactopyranoside (IPTG) to a final concentration of 1 mM and the addition rate of the glycerol feed was lowered (45% aq glycerol at a feed rate of 0.973 kg/h). The time from induction until harvest was called the induction phase or product formation phase.

The induction phase was stopped by cooling the fermenter medium to about 18±2° C. and samples were taken for microscopic analysis. The biomass was recovered by centrifugation (continuous CEPA centrifuge with 8 L bowl at a flow rate of 40 to 100 L/h) and centrifuged at 18,000×g while cooling to 18° C.±2° C. The cell paste was resuspended in a cell disruption buffer (48 mM Tris, 2 mM EDTA, 96 mM NaCl, pH 7.4) at ≤15° C. and resuspended cells were disrupted by two cycles of high-pressure homogenization at 950±50 bar (SPX homogenizer). During the homogenisation steps the product containing solution was constantly cooled.

After cell disruption, inclusion bodies (IBs) were recovered by centrifugation as before and soluble impurities were washed (200 L stirred tank) by five consecutive washing cycles using different buffers: Wash 1 and 2, 50 mM Tris, 2 mM EDTA, 1.5M NaCl, 0.2% polysorbate 80, pH 7.4; Wash 3 and 4, 50 mM Tris, 2 mM EDTA, 100 mM NaCl, 2% polysorbate 80, 20 mM DTT, pH 7.4; Wash 5, 50 mM Tris, 2 mM EDTA, 100 mM NaCl, 20 mM DTT, pH 7.4. After the washes were completed, the inclusion bodies were stored at ≤−60° C.

### Solubilization and Refolding of Inclusion Bodies

For solubilization and further processing, one half of the washed inclusion bodies (˜1 kg) were removed from storage and thawed at 2-8° C. (1-3 days). The IBs were resuspended in a total of 25 L of solubilization buffer (50 mM Tris, 2 mM EDTA, 6M Guanidine, 100 mM NaCl, pH 7.4). Dispersion was accomplished using and ULTRA-TURRAX (IKA) with a S50N-G45G probe at 5 000 to 8 000 rpm for 8-10 min followed by S50N-G45F probe at 7 000 to 10 000 rpm for an additional 8-10 min and finally stirring was done for 60 min. After resuspension of the IBs, DTT was added to a final concentration of 50 mM and disulfide bonds were reduced to thiols for 15 hours. DTT was then removed again via ultrafiltration using a Pall Centrasette system (10 kDa Sartocon with PES membrane, Sartorius Stedim Biotech, Part #3021463907E). The product is then slowly added to 550 L of refolding buffer (1M Arginine hydrochloride, 0.1M NaCl, 5 mM EDTA, 30 mM NaOH, 1 mM GSSG; 5 mM GSH, pH 7.4) and incubated for 9 hours. Finally, the product is clarified by a depth filtration and is subsequently filtered over a 0.2 μm bioburden reduction membrane (Sartopore 2 XLG capsule; 0.8/0.2 μm retention rate; filter membrane made of PES, Sartorius Stedim Biotech, Part #5445307G) into 500 L bags.

The filtered refolded protein solution containing the modified FGF-1 polypeptide (SEQ ID NO: 2) was subsequently used for further downstream processing, as described below.

### Chromatographic Purification and Filling of Bulk Drug Substance

The refolded FGF-1 polypeptide (SEQ ID NO: 2) was captured using a column (15.6 L volume) packed with Capto DeVirs (Cytiva, BPG 300×500, Part #17-5466) after 1:1 dilution with water to ensure binding. The column was eluted with a linear gradient from 0% to 100% Buffer 1B (corresponding to 100% to 0% of Buffer 1A), at a flow rate of 21.2-179.9 L/h run at room temperature. Following elution, the column was held at 100% Buffer 1B over 5 CV. Buffer 1B=78.13 g/L sodium chloride; 0.20 g/L potassium chloride; 1.78 g/L di-sodium hydrogenphosphate dihydrate; 0.27 g/L Potassium dihydrogen phosphate; pH 7.4 (corresponding to 100% to 0% Buffer 1A: 8.00 g/L sodium chloride; 0.20 g/L potassium chloride; 1.78 g/L di-sodium hydrogenphosphate dihydrate; 0.27 g/L Potassium dihydrogen phosphate; pH 7.4). Buffer 1A=8.00 g/L sodium chloride; 0.20 g/L potassium chloride; 1.78 g/L di-sodium hydrogenphosphate dihydrate; 0.27 g/L Potassium dihydrogen phosphate; pH 7.4. Buffer 1B is 78.13 g/L sodium chloride; 0.20 g/L potassium chloride; 1.78 g/L di-sodium hydrogen phosphate dihydrate; 0.27 g/L Potassium dihydrogen phosphate; pH 7.4.

The eluate was collected in a single fraction. The collected eluate was filtered with a 0.2 μm Filter (Sartopore 2 XLG Art. No.: 5445307G8) and stored at 15-25° C. for less than 24 h. The yield after capture step using the Capto DeVirs column was approximately 120 g of the modified FGF-1 polypeptide (SEQ ID NO: 2), at a concentration of about 1.74 g/L of the eluate.

Following this column step, the product was conditioned for polishing by addition of ammonium sulfate (1.0 M final concentration) and sodium chloride (2.5 M final concentration). Polishing was accomplished by hydrophobic interaction chromatography (HIC) using a column (10.6 L volume) packed with Butyl Sepharose 4FF (Cytiva, BPG 300×500, Part #17-0980) and equilibrated in 20 mM di-sodium hydrogenphosphate dihydrate; pH 7.4. The column was washed with three bed volumes of the same buffer. The product was eluted with a linear gradient from 0% to 100% Buffer 2B (corresponding to 100% to 0% Buffer 2A) at a flow rate of 51.3 L/h run at room temperature over 10 CV. Buffer 2A=20 mM di-sodium hydrogen phosphate dihydrate; pH 7.4 and Buffer 2B=2.5 M sodium chloride, 1 M ammonium sulfate, 20 mM di-sodium hydrogen phosphate dihydrate, pH 7.4. The eluate was transferred to a Stedim bag (1×50 L). The yield after the polishing step was about 92 g of the modified FGF-1 polypeptide (SEQ ID NO: 2), at a concentration of 3 g/L. The yield at the polishing step using the Butyl Sepharose FF column was approximately 50%, a significant improvement compared to an yield of approximately 20% by using a heparin resin for the polishing step.

The bulk drug substance (BDS) purified according to the above method was stored as a solution in the eluate buffer of the Butyl Sepharose FF column. The modified FGF-1 polypeptide (SEQ ID NO: 2) was observed to elute with a gradient and therefore, the buffer had no precise composition but contained approximately 800 mM sodium chloride, 320 mM ammonium sulfate and 20 mM di-sodium hydrogen phosphate dihydrate at pH 7.4.

The purified BDS was filtered (Millipak 100 Gamma Gold, 0.22 m, Merk, Part #MPGL1GCF3) into a second Stedim sterile bag using a peristaltic pump. The overall yield of the bulk drug substance from the above described process was about 82 g, from a 50 L culture.

The filling of the drug substance as prepared above was performed by aseptically dispensing the filtered protein solution into Gamma-sterilized, pyrogen free, polyethylene bottles with high-density polyethylene screw caps. Aliquots of the bulk drug substance were stored at a temperature of about −60° C.±10° C.

### Example 4: Pharmaceutical Formulation

An exemplary generalized study for assessing different variations of the pharmaceutical formulation comprising a modified FGF-1 polypeptide of SEQ ID NO: 2, included a comparison of 10 mM versus 1 mM Histidine buffers at three different pH values across two different surfactant conditions, to identify a pharmaceutical formulation of optimized stability and reduced tendency of degradation. Stability was determined by two methods such as scaled visual inspection, size-exclusion HPLC (SE-HPLC), FlowCam analysis. Visual inspection was performed under a white light source (13 W fluorescent tube) against black and white backgrounds. Digital photographs were acquired of all formulations at every time point. A FlowCam particle imaging system combines optics, electronics, and fluidics for automated analysis of particles. The optical system is used to capture real-time images of the particles in the fluid as they pass through the flow cell. The imaging software provides the ability to assess particle size and morphology. All samples were degassed for 30 minutes at 75 torr then analyzed neat.

For instance, a scaled visual inspection method was used to observe drug precipitation and SE-HPLC analysis was used to observe drug degradation and high molecular weight (HMW) species formation. After preparation of the pharmaceutical formulations, final formulations were stored on a benchtop in a covered tube rack at room temperature (approximately 20° C.). Visual inspections and SE-HPLC analyses were performed on days 0, 5, 14, 28, and 59 after preparation.

In an exemplary study, visible precipitation occurs soon after preparation in samples wherein pH above 6.5. Irrespective of histidine concentration, samples with pH of 5.8 are better at keeping drug in solution and particles are not seen in the 10 mM histidine buffer until 59 days after preparation, and only one suspended particle is observed in the 1 mM histidine buffer at day 28. All PS20 formulations are not inspected after day 5 as they all recover lower than their PS80 counterparts at Day 5 by SE-HPLC.

Two rounds of surfactant screen were conducted. In the first round, the modified FGF-1 polypeptide (SEQ ID NO: 2) at a concentration of about 0.1 mg/mL (100 μg/mL) was formulated in 10 mM citrate, 300 mM NaCl, at pH 6. Various surfactants were tested—and the FGF-1 formulation was spiked with surfactant stock solutions to obtain final concentrations as follows: 0.1% (w/v) Tyloxapol, 0.01% (w/v) Polysorbate 80 (PS80), and 0.1% (w/v) Poloxamer 188 (F-68). The FGF-1 formulation without surfactant was used as a control. In a separate round of surfactant screening, the FGF-1 polypeptide (SEQ ID NO: 2) was formulated at a concentration of about 0.25 mg/mL (250 μg/mL) in 1.046 M NaCl, 0.419 M ammonium sulfate, 20 mM disodium hydrogen phosphate dihydrate, pH 7.4 (butyl eluate). For the second round of surfactant screen, the modified FGF-1 polypeptide (SEQ ID NO: 2) in either the formulation containing 10 mM citrate and 300 mM NaCl or the butyl eluate formulation were dialyzed into various formulations (see Table 6) and following dialysis the concentration of the drug substance was reduced from 0.25 mg/mL to about 0.1 mg/mL in respective formulation buffers and filled into Crystal Zenith vials at a volume of about 1.25 mL. Following table provides the details for the surfactant screen round two.

TABLE 6
Formulation for Surfactant Screen, Round 2

| | Concen- | | Drug |
|---|---|---|---|
| Surfactant | tration | Buffer | Substance |
| None | 100 μg/mL | 10 mM Histidine, | Original DS (10 |
| 0.01% PS20 | | 5% Sorbitol, pH 5.8 | mM citrate, 300 mM |
| 0.10% PS20 | | | NaCl) |
| 0.05% PS80 | | | New DS (butyl |
| 0.10% PS80 | | | eluate) |
| 0.30% F-68 | | | |
| 0.50% F-68 | | | |
| None | 100 μg/mL | 10 mM Histidine, | Original DS (10 |
| 0.01% PS20 | | 150 mM NaCl, | mM citrate, 300 mM |
| 0.10% PS20 | | pH 7.0 | NaCl) |
| 0.05% PS80 | | | New DS (butyl |
| 0.10% PS80 | | | eluate) |
| 0.30% F-68 | | | |
| 0.50% F-68 | | | |

The sample vials from each formulation were tested by exposure to agitation stress on a shaker for about 4 hours at 1000 rpm at ambient temperature and a vial of the same formulation was used as a static unstressed control (stored for 4 hours at ambient temperature). Following the 4-hour agitation stress, stressed samples were subjected to five cycles of freezing and thawing (stored at −20° C. until frozen, then placed at ambient temperature until thawed). Unstressed samples were stored at 5° C. for the duration of the freeze-thaw cycles. Following the conclusion of agitation and freeze-thaw stresses, samples were analyzed by visual inspection and FlowCam.

For the first round of surfactant screening, all static samples, with and without surfactant, were clear, colorless, and displayed a few fine particulates. Surfactant-free samples that were subjected to agitation and freeze-thaw cycling appeared slightly opaque and contained many fine particles. Samples containing 0.1% tyloxapol or 0.100 F-68 were clear, colorless, and exhibited a white flaky precipitate upon agitation and freeze-thaw cycling. While stressed samples containing 0.01% PS8O were clear and colorless with many fine particles. In FlowCam analysis, all static samples displayed moderate subtracted subvisible particle counts. Agitated samples containing 0.1% F-68 displayed the lowest particle counts compared to agitated samples of other surfactant conditions. However, stressed F-68 samples still showed significant increases in subvisible particles. Agitating and freeze-thawing samples without surfactant gave rise to the highest subvisible particle concentrations.

For the second round of surfactant screening, all static samples without surfactant were clear, colorless, and exhibited a few visible particles. The New DS (butyl eluate) sorbitol sample was clearer in appearance than the other samples and exhibited many fine bubbles in solution. All static samples containing PS20, except New DS (butyl eluate) with NaCl and 0.1% PS20, were clear, colorless, and free of visible particles. The New DS NaCl sample containing 0.1% PS20 was clear, colorless, and exhibited a few visible particles. Agitated samples containing sorbitol generally were clear, colorless, and exhibited a few visible particles. Agitated samples containing NaCl generally were slightly opaque in appearance and exhibited visible particles.

All static samples containing PS80 were clear, colorless, and generally free of visible particles. Agitated samples containing sorbitol generally were clear, colorless, and exhibited a few visible particles. Agitated samples containing NaCl generally were slightly opaque in appearance and exhibited visible particles All static samples containing F-68 were clear, colorless, and free of visible particles. All agitated samples containing F-68 were clear, colorless, and exhibited a few visible particles. With respect to FlowCam analysis after second round of surfactant screen—formulations with sorbitol generally displayed lower subtracted subvisible particle concentrations relative to formulations with NaCl. The new drug substance (butyl eluate) exhibited significantly lower subvisible particle concentrations following stress, across all surfactant conditions, compared to the old drug substance (citrate formulation), regardless of formulation (sorbitol vs. NaCl). Overall, the new DS (butyl eluate) in the sorbitol formulation containing 0.10% PS80 exhibited the least amount of change in subvisible particle content following stress.

In the SE-HPLC analysis, the new DS (butyl eluate) in sorbitol formulation and either 0.05% or 0.1% PS80 displayed comparable profiles under static and stressed conditions. All new DS (butyl eluate) samples in sorbitol formulation with 0.05% or 0.10% PS80 displayed one HMW peak (% area of about 7% for peak 1 and 92-93% for the main peak) compared to the new DS (butyl eluate) samples containing sorbitol and PS20 that exhibited two HMW species (% area of about 0.5% for HMW peak 1, 7.8-8% for HMW peak 2, and about 91.6-91.8% for the main peak).

An accelerated stability study was subsequently carried out with various pharmaceutical formulations for the modified FGF-1 polypeptide of SEQ ID NO: 2. The Accelerated Stability study evaluated the formulations by various assays over 8 weeks while stored at five different storage temperatures. Data was collected at initial time point (time zero), acute stresses, and the final time point (T=2 weeks for 40° C. and T=8 weeks for all other temperatures). A pH variation study was also carried out as part of the pharmaceutical formulation evaluation and optimization study. The conclusion from the accelerated stability studies was that low pH is optimal for the pharmaceutical formulation comprising a modified FGF-1 polypeptide of SEQ ID NO: 2 in Histidine/Polysorbate/Sorbitol formulations and this was supported in two ways by visual inspection, FloCam analysis, and SE-HPLC. In SE-HPLC, for instance, main peak was higher in low pH samples, indicating less aggregation and/or degradation at low pH levels, leaving more intact monomer to be detected. Whereas, samples at the higher pH levels had higher percentages of soluble aggregates at each timepoint than samples at lower pH's. These increases in HMW species over time also seem to worsen as pH increased. In other words, a positive correlation was observed between pH and HMW species presence over time.

In a further study, the concentration of histidine in the formulation was reduced from 10 mM to 1 mM and the effects of that change on HMW peak area percentage was assessed using SE-HPLC. As provided in Table 8, the formulations at pH 5.8, regardless of histidine concentration, demonstrated lower HMW peaks, compared to the formulations at pH greater than 6.0.

The different variations of the candidate formulation e.g., 10 vs. 1 mM Histidine buffers at three different pH values across two different surfactant conditions were compared (Table 7). The parameters compared were chosen to determine if pH would still be controlled in 1 mM histidine buffers as opposed to 10. The primary goal of comparing these formulations was to deduce whether the promising candidate formulation, and/or any of its variations, provided suitable stability of up to 1 month for 100 (±20%) μg/mL TTHX1114 formulations at room temperature. Secondarily, it was desirable to learn whether the same degree of stability could be achieved in variations where the histidine concentration was lowered and/or the pH was raised slightly as these adjustments could potentially lead to a more soothing eyedrop upon application.

TABLE 7
Set up of samples for stability tests

| Surfactant | Histidine Conc. | pH (±0.2) |
|---|---|---|
| 0.1% PS80 | 10 mM Histidine | pH 5.8 |
| | | pH 6.5 |
| | | pH 7.0 |
| | 1 mM Histidine | pH 5.8 |
| | | pH 6.5 |
| | | pH 7.0 |
| 0.1% PS20 | 10 mM Histidine | pH 5.8 |
| | | pH 6.5 |
| | | pH 7.0 |
| | 1 mM Histidine | pH 5.8 |
| | | pH 6.5 |
| | | pH 7.0 |

Stability was determined by: (i) a scaled visual inspection method to observe drug precipitation and (ii) SE-HPLC analysis to observe drug degradation and HMW species formation. After preparation, final formulations were stored on the benchtop in a covered tube rack at room temperature (approx. 20° C.). Visual inspections and SE-HPLC analyses were performed on days 0, 5, 14, 28, and 59 after preparation. Formulations that did not perform well at a particular timepoint were excluded from analysis at subsequent timepoints. Additionally, the pH was measured again at day 59 in remaining formulations to ensure that no drift had occurred over the course of the study.

### Diluent and Stock Solution Preparation

Two base diluents lacking L-Histidine were prepared: Diluent A (0.2% PS80/10% sorbitol) and was prepared by dissolving PS80 and sorbitol in deionized (DI) H₂O. This was diluted to 500 mL with DI H₂O and then filtered through a 0.2 μm polyethersulfone (PES) membrane vacuum filter unit. The second base diluent was Diluent B (0.2% PS20/10% sorbitol) and was prepared by dissolving PS20 and sorbitol in DI H₂O. This was diluted to 500 mL with DI H₂O and then filtered through a 0.2 μm PES membrane vacuum filter unit.

After preparing the base diluents, two different concentrated stock solutions of dialysis buffers were prepared. Stock A (9.29 mM Histidine/0.929% PS80/46.43% Sorbitol) was prepared by adding and dissolving Sorbitol, PS80, and L-Histidine in DI H₂O. This was diluted to 3.5 L with DI H₂O and 2 L of this was filtered through a bottle-top 0.2 μm PES membrane vacuum filter into a sterilized 2 L bottle. Stock B (9.18 mM Histidine/0.909% PS20/45.45% Sorbitol) was prepared by adding and dissolving Sorbitol, PS80, and L-Histidine in DI H₂O. This was diluted to 2750 mL with DI H₂O and 2 L of this was filtered through a bottle-top 0.2 μm PES membrane vacuum filter into a separate, sterilized 2 L bottle.

After removing the modified FGF-1 polypeptide (SEQ ID NO: 2) aliquot from storage at −70° C. freezer was allowed to thaw for 1 hour at room temperature while the stock solutions were diluted to dialysis buffers that were at the desired 1× concentration: 2 mM Histidine/0.2% Polysorbate 80 or 20/10% Sorbitol. The thawed sample was mixed well and O.D was measured at 280 nm. Dialysis was performed at room temperature (RT) with a 1:100 ratio of sample to dialysis buffer (30 mL of sample: 3000 mL of dialysis buffer) and with 2 subsequent buffer changes. The first change was performed 2 hours after the start time, the second change was performed 4 hours after the start time. After the second change, the sample was allowed to dialyze overnight at RT (13 more hours). TTHX114 was then formulated in 2 mM histidine/0.2% PS80 or 20/10% sorbitol.

Both dialyzed samples were split into two portions (2A.1+2A.2 and 2B.1+2B.2). To 2A/B.1 solutions, the concentration of histidine was increased to 20 mM by adding Diluent A or B that was supplemented with 10×, 180 mM, histidine. These 10× histidine solutions were prepared by adding 1.117 g of L-Histidine to each of 40 mL of Diluent A and B.

For solution 2A.1, 1.6 mL of 10× histidine in Diluent A solution was added to 14.4 mL of dialyzed sample. For solution 2B.1, 1.8 mL of 10× histidine in Diluent B was added to 14.6 mL of dialyzed sample followed by 1.6 mL of Diluent B. In addition to bringing the histidine concentration up to 20 mM in the 2A.1 and 2B.1 solutions, these dilutions also brought TTHX1114 concentrations to 200 μg/mL in those portions. The other portions of both dialyzed samples (2A/B.2 solutions) were simply diluted to 200 μg/mL TTHX1114 with Diluent A or B that had been supplemented with 2 mM histidine (which was prepared by diluting the 180 mM solutions 1:90 with respective diluents). 2A.1+2A.2 and 2B.1+2B.2 are 2× in all components. The samples were filtered through 0.2 am cellulose acetate syringe filters. Visual inspection for particulate suspension was done and ranked in a scale from 0-3 and a single rank of >3, where 0 signifies no visible suspended particulate matter.

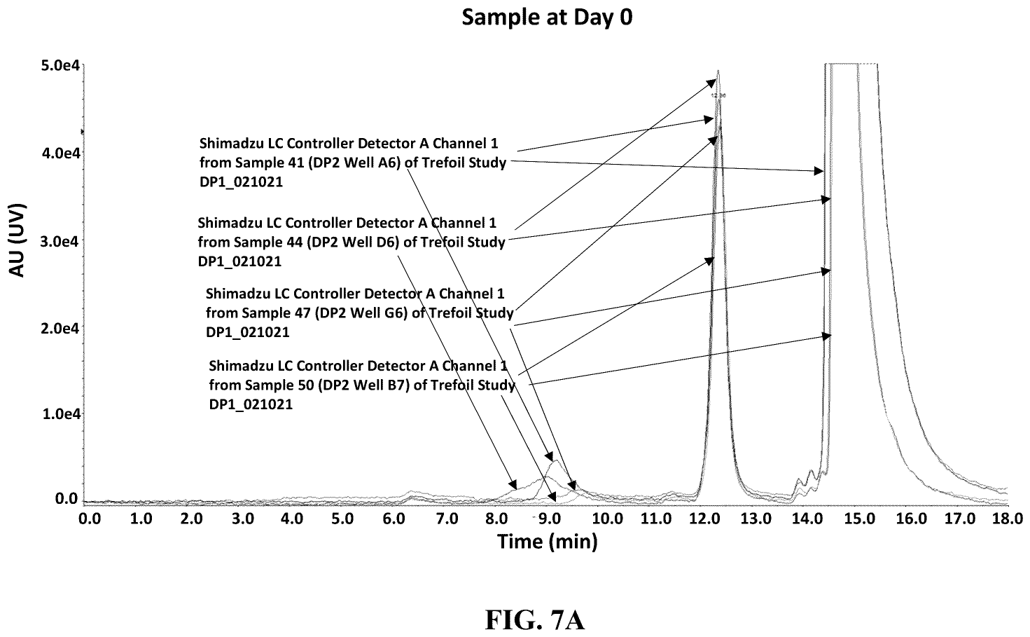

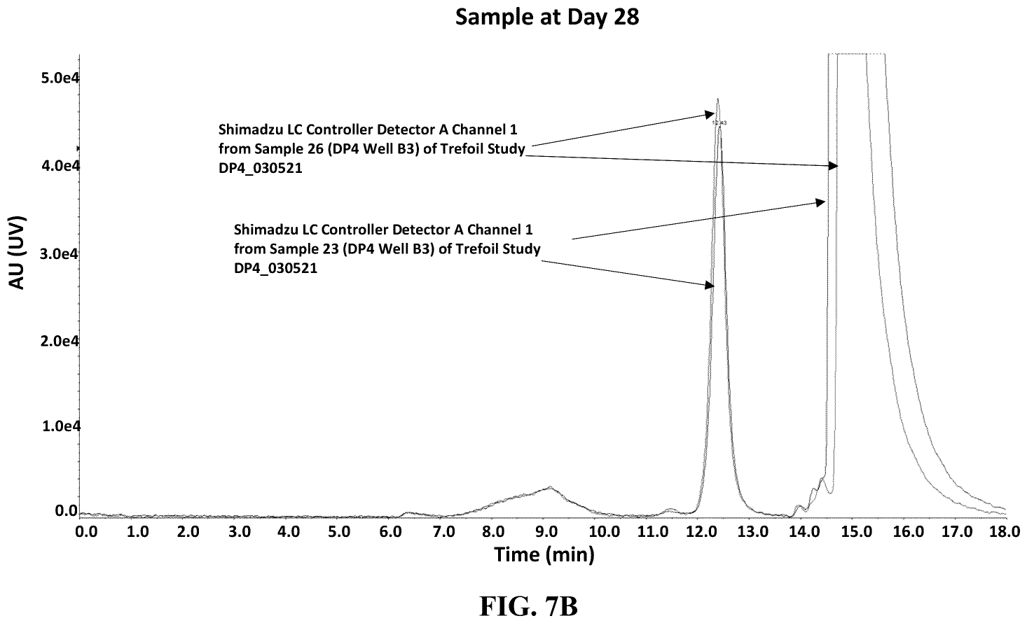

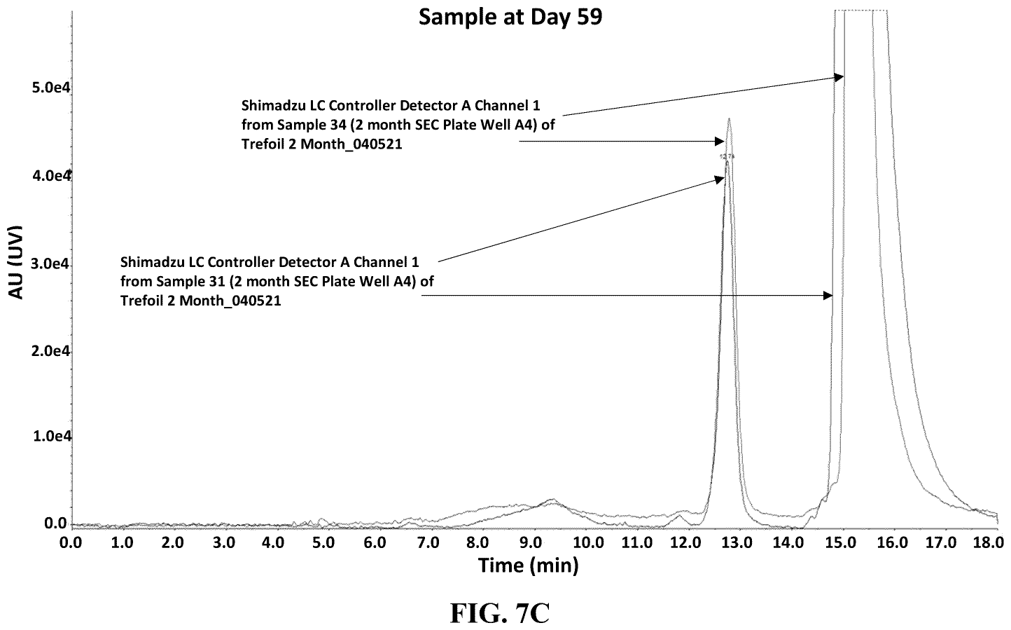

Results: Irrespective of histidine concentration, pH 5.8 samples were better at keeping drug in solution and particles were not seen in the 10 mM histidine buffer until 59 days after preparation, and only one suspended particle was observed in the 1 mM histidine buffer at day 28. All PS20 formulations were not inspected after day 5 as they all recovered lower than their PS80 counterparts at Day 5 by SE-HPLC. Representative SE-HPLC peak is shown in FIG. 7.

TABLE 8
Summary of histidine concentration, pH variation study over time

| | HMW as Percentage of Total Peak Area (%) | | | | |
|---|---|---|---|---|---|
| | Day | Day | Day | Day | Day |
| Formulation ID | 0 | 5 | 14 | 28 | 59 |
| 10 mM His/0.1% PS80/5% | 0.32 | 0.41 | 1.29 | 2.20 | 2.44 |
| sorbitol, pH 5.8 | 0.76 | 1.66 | 3.65 | — | — |
| 10 mM His/0.1% PS80/5% | 0.96 | 2.91 | 7.62 | — | — |
| sorbitol, pH 6.5 | | | | | |
| 10 mM His/0.1% PS80/5% | | | | | |
| sorbitol, pH 7.0 | | | | | |
| 1 mM His/0.1% PS80/5% | 0.58 | 0.54 | 0.44 | 0.08 | 1.09 |
| sorbitol, pH 5.8 | 0.72 | 1.89 | 3.24 | — | — |
| 1 mM His/0.1% PS80/5% | 2.04 | 3.41 | 6.73 | — | — |
| sorbitol, pH 6.5 | | | | | |
| 1 mM His/0.1% PS80/5% | | | | | |
| sorbitol, pH 7.0 | | | | | |

### Maintaining pH

After adjusting to the requisite pH's on Day 0 (see Table 6) the pH was evaluated again at day 59 to ensure that there was no drift in the formulations' pH's. Samples at both histidine concentrations held their pH's well over the course of the study and no significant drift was observed.

| Buffer ID | Target pH | Day 0 pH | Day 59 pH |
|---|---|---|---|
| 3A. 1 | 5.8 | 5.83 | 5.84 |
| 3A. 4 | 5.8 | 5.77 | 5.73 |

Overall, the study elucidated a formulation in which the drug product (FGF-1 polypeptide of SEQ ID NO: 2) is stable for at least 28 days at RT. An optimized formulation is a 10 mM histidine/0.1% PS80/5% sorbitol formulation at pH 5.8 due to the lack of visible particulates for an extended period of time, the nearly constant main peak area by SE-HPLC, the low percentage of soluble aggregate over time by SE-HPLC, and the control of pH over time. Similar trends were observed for the formulation containing 1 mM histidine/0.1% PS80/5% sorbitol making it another formulation in which the drug product (FGF-1 polypeptide of SEQ ID NO: 2) was found to be stable for at least 59 days.

### Example 5. Plasmid, Cloning and Expression of Modified Human FGF-1

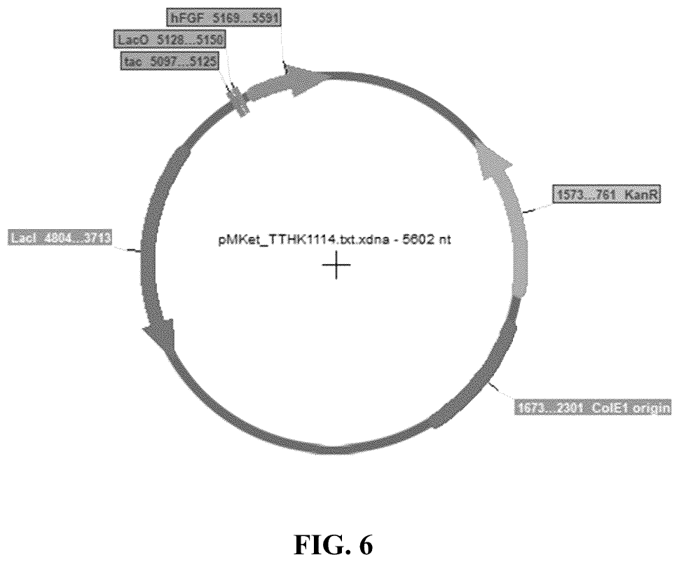

This example describes the production of modified FGF-1 polypeptide (SEQ ID NO: 2) in bacteria. To allow expression of engineered (modified) human FGF-1 (TTHX1114) in E. coli (BL21 competent cells) the encoding sequence of the target protein was optimized for E. coli expression including codon usage, transcription and translation efficiency and mRNA stability and de-novo synthesized by GenScript and subcloned into the expression plasmid. The plasmid map for the selected plasmid TTHX1114_pMKet is shown in FIG. 6. The designed construct encodes the genetic information of the modified FGF-1 polypeptide without any terminal fusion.

For periplasmic accumulation of the modified FGF-1 polypeptide, the mature protein was fused at the N-terminus by the leader peptide of the outer membrane protein (ompA, oA). This leader sequence is required for translocation of the pre-protein into the periplasmic space, that is cleaved post-translocation via the signal peptidase I (SP-1). The plasmid has a sequence conferring Kanamycin resistance. The inserts were subcloned into a plasmid backbone TTHX1114_pMKet. The periplasmic construct was subcloned into pMKet backbone (plasmid name: oA-TTHX1114_pMKet). The expression of the modified FGF-1 polypeptide was controlled by the Tac promoter, which is an IPTG dependent promoter.

The periplasmic construct was designed in order to ensure after screenings the insertion of additional sequences downstream of the modified FGF-1 ORF in order to allow bi-cistronic co-expression of e.g., chaperone (sequence between two stop codons (underlined) and BamHI restriction site (italicized) in the exemplary codon optimized nucleotide sequence for the engineered hFGF-1 is provided below (length: 438 bp, including flanking sites):

(SEQ ID NO: 207)
CATATGAAAAAGACAGCTATCGCGATTGCAGTGGCACTGGCTGGTTTCGC
TACCGTTGCGCAAGCTTTTAATCTGCCGCCGGGTAACTATAAGAAACCGA
AACTGTTGTACAGCTCTAATGGTGGCCACTTCCTGCGTATCCTGCCGGAC
GGCACCGTCGATGGTACCCGTGACCGCAGCGATCAACACATTCAACTGCA
ACTGAGCGCCGAGAGCGTGGGCGAAGTTTACATTAAGTCCACTGAAACGG
GCCAGTACCTGTGTATGGACACCGATGGCCTGCTGTACGGTTCGCAGACG
CCAAATGAAGAGTGCCTGTTCTTGGAGCGTCTGGAAGAGAACCACTATAA
CACCTACATTAGCAAGAAACATGCGGAGAAAAACTGGTTTGTGGGTCTGA
AGAAAAATGGTTCCGTCAAGCGCGGTCCTCGTACGCATTATGGCCAGAAA
GCAATCTTGTTCCTGCCGCTGCCGGTTAGCAGCGACTAATAGAAGGAGAT
ATAGCCATGGTCTGGACAGAAGCTTGGATCC

Exemplary sequence of the modified FGF-1 polypeptide for periplasmic accumulation is provided below:

(SEQ ID NO: 208)
MKKTAIAIAVALAGFATVAQAFNLPPGNYKKPKLLYSSNGGHFLRILPDG
TVDGTRDRSDQHIQLQLSAESVGEVYIKSTETGQYLCMDTDGLLYGSQTP
NEECLFLERLEENHYNTYISKKHAEKNWFVGLKKNG
SVKRGPRTHYGQKAILFLPLPVSSD.

Post-translocation, the mature FGF-1 polypeptide of SEQ ID NO: 2 was generated.

### Embodiments

Embodiment 1 provides a formulation, comprising:
(a) a modified FGF-1 polypeptide comprising an amino acid sequence set forth in SEQ ID NO: 1, or having an amino acid sequence that is at least 90% identical to SEQ ID NO: 1, and comprising at least 1, 2, 3, 4 or 5 single amino acid mutations; and
(b) L-methionine.

Embodiment 2 provides the formulation of embodiment 1, wherein the formulation is an injectable formulation for intraocular delivery.

Embodiment 3 provides the formulation of embodiment 1, wherein the modified FGF-1 polypeptide comprises an N-terminal methionine residue positioned upstream to the first residue of SEQ ID NO: 1.

Embodiment 4 provides the formulation of embodiment 1, wherein the polypeptide further comprises an extension peptide positioned between the N-terminal methionine residue and the first residue of SEQ ID NO: 1.

Embodiment 5 provides the formulation of embodiment 4, wherein the extension peptide comprises one or more amino acid residues of SEQ ID NO: 3, or comprises any one of the sequences set forth in SEQ ID NOS. 4-8.

Embodiment 6 provides the formulation of any one of the embodiments 1-5, wherein the modified FGF-1 polypeptide is the mature form of the polypeptide.

Embodiment 7 provides the formulation of any one of the embodiments 1-6, wherein the modified FGF-1 polypeptide comprises a sequence selected from SEQ ID NOS: 14-18.

Embodiment 8 provides the formulation of any one of the embodiments 1-6, wherein the modified FGF-1 polypeptide comprises a sequence selected from SEQ ID NOS: 24-28.

Embodiment 9 provides the formulation of any one of the embodiments 1-6, wherein the modified FGF-1 polypeptide comprises a sequence selected from SEQ ID NOS: 93-117.

Embodiment 10 provides the of any one of the embodiments 1-6, wherein the polypeptide further comprises a methionine residue N-terminal to the extension peptide.

Embodiment 11 provides the formulation of any one of the embodiments 1-6, wherein the modified FGF-1 polypeptide comprises a sequence selected from SEQ ID NOS: 118-141.

Embodiment 12 provides the formulation of any one of the embodiments 1-11, wherein the modified FGF-1 polypeptide is expressed in a form that comprises 136 amino acids.

Embodiment 13 provides the formulation of any one of the embodiments 1-11, wherein the modified FGF-1 polypeptide comprises at least 141 amino acids in its mature form.

Embodiment 14 provides the formulation of any one of the embodiments 1-13, wherein the modified FGF-1 polypeptide comprising a mutation at position 67 of SEQ ID NO: 1.

Embodiment 15 provides the formulation of any one of the embodiments 1-14, wherein the modified FGF-1 polypeptide further comprises a truncation of one or more of the first five residues of SEQ ID NO: 1.

Embodiment 16 provides the formulation of any one of the embodiments 1-15, wherein the modified FGF-1 polypeptide comprises a sequence selected from SEQ ID NOS: 146-149.

Embodiment 17 provides the formulation of any one of the embodiments 1-16, wherein the polypeptide further comprises an extension peptide comprising one or more amino acid residues of SEQ ID NO: 3.

Embodiment 18 provides the formulation of any one of the embodiments 1-16, wherein the modified FGF-1 polypeptide comprises a sequence selected from SEQ ID NOS: 174-204.

Embodiment 19 provides the formulation of any one of the embodiments 1-18, wherein the modified FGF-1 polypeptide comprising a sequence as set forth in SEQ ID NO: 2, or SEQ ID NO: 205.

Embodiment 20 provides the formulation of embodiment 19, wherein the modified FGF-1 polypeptide is the mature form of the polypeptide.

Embodiment 21 provides the formulation of any one of the embodiments 1-20, wherein the modified FGF-1 polypeptide comprises one or more mutations selected from the group consisting of: Cys16Ser, Ala66Cys, and Cys117Val.

Embodiment 22 provides the formulation of any one of the embodiments 1-20, wherein the modified FGF-1 polypeptide comprises one or more mutations of SEQ ID NO: 1, said mutation is selected from the group consisting of: Lys12Val, Cys16Ser, Ala66Cys, Cys 117Val, and Pro134Val, and wherein the modified FGF-1 polypeptide further comprises at least one residue of the peptide ALTEK.

Embodiment 23 provides the formulation of any one of the embodiments 1-22, wherein the modified FGF-1 polypeptide comprises one or more mutations comprising the following mutations of SEQ ID NO: 1: Cys16Ser, Ala66Cys, and Cys117Val, wherein the modified FGF-1 polypeptide comprises a methionine residue positioned upstream to the first residue of SEQ ID NO: 1, and at least one residue of the peptide ALTEK located between the N-terminal methionine and position 1 of SEQ ID NO: 1.

Embodiment 24 provides the formulation of embodiment 1, wherein the formulation comprises human serum albumin (HSA) and/or polysorbate 80.

Embodiment 25 provides the formulation of embodiment 1, wherein the modified FGF-1 polypeptide is at greater than 95% pure monomeric form in the formulation.

Embodiment 26 provides the formulation of embodiment 25, further comprising at least one of:
a. at least about 50 mM dibasic sodium phosphate dihydrate;
b. at least about 100 mM sodium chloride;
c. at least about 10 mM ammonium sulfate;
d. at least about 0.1 mM ethylenediaminetetraacetic acid (EDTA);
e. at least about 5 mM L-Methionine, and
f. at least about 0.01% polysorbate 80 (w/v).

Embodiment 27 provides the formulation of embodiment 26, wherein the formulation comprises the EDTA at a concentration of the EDTA is from at least about 0.01 mM to about 10 mM.

Embodiment 28 provides the formulation of embodiment 26, wherein the formulation comprises the ammonium sulfate, and wherein the concentration of the ammonium sulfate is from at least about 0.01 mM to about 100 mM.

Embodiment 29 provides the formulation of embodiment 26, wherein the formulation comprises the L-Methionine is from at least about 0.01 mM to about 100 mM.

Embodiment 30 provides the formulation of embodiment 1, wherein the modified FGF-1 is present at a concentration suitable for treating one or more diseases, disorders, or conditions selected from a list consisting of: Fuch's dystrophy, bullous keratopathy, herpetic keratopathy, congenital hereditary endothelial dystrophy 1, congenital hereditary endothelial dystrophy 2, posterior polymorphous corneal dystrophy, a dry eye syndrome, keratoconus, lattice corneal dystrophy, granular corneal dystrophy, macular corneal dystrophy, Schnyder crystalline corneal dystrophy, congenital stromal corneal dystrophy, fleck corneal dystrophy, corneal injury, ocular injury, chemical injury, vesicant injury, stromal injury and mustard gas keratopathy.

Embodiment 31 provides the formulation of embodiment 1 or 30, wherein the formulation is administered intracamerally.

Embodiment 32 provides the formulation of embodiment 1 or 30, wherein the formulation is administered intravitreally.

Embodiment 33 provides the formulation of any one of the embodiments 1-31, wherein the formulation is stable for at least about 2 weeks to about 4 weeks, at a temperature of about −20° C.

Embodiment 34 provides a scalable method for producing a therapeutically effective modified FGF-1 polypeptide, the method comprising:
a. introducing a recombinant nucleic acid construct in a suitable E. coli cell, wherein the recombinant nucleic acid construct comprises a sequence encoding the modified FGF-1 polypeptide for cytoplasmic expression, inserted in vector comprising a pBR322 derived ori-sequence,
b. growing the cells in a synthetic growth media comprising a suitable antibiotic for about 20 hours; and
c. recovering from the cell, a therapeutically effective modified FGF-1 polypeptide,
wherein the yield of the modified FGF-1 recovered at step c is at least 2-fold higher than a method that does not comprise using a vector comprising a pBR322 derived ori-sequence, the synthetic growth media, or a combination thereof.

Embodiment 35 provides the method of embodiment 34, wherein the modified FGF-1 polypeptide comprises an amino acid sequence that is at least 90% identical to the amino acid sequence of SEQ ID NO: 1; or a sequence that comprises one or more mutations of at positions 12, 16, 66, 117, and 134 of SEQ ID NO: 1.

Embodiment 36 provides the method of embodiment 34, wherein the modified FGF-1 polypeptide comprises one or more of (i) Ala66Cys mutation, (ii) Cys16Ser mutation, (iii) Cys 117Ser mutation.

Embodiment 37 provides the method of embodiment 34, wherein the modified FGF-1 polypeptide comprises an N-terminal methionine residue positioned upstream to the first residue of SEQ ID NO: 1.

Embodiment 38 provides the method of embodiment 34, wherein the modified FGF-1 polypeptide further comprises an extension peptide positioned between the N-terminal methionine residue and the first residue of SEQ ID NO: 1.

Embodiment 39 provides the method of embodiment 38, wherein the extension peptide comprises one or more amino acid residues of SEQ ID NO: 3.

Embodiment 40 provides the method of embodiment 39, wherein the extension peptide comprises any one of the sequences set forth in SEQ ID NOS. 4-8.

Embodiment 41 provides the method of any one of the embodiments 34-40, wherein the modified FGF-1 polypeptide is the mature form of the polypeptide.

Embodiment 42 provides the method of any one of embodiments 34-41, wherein the modified FGF-1 polypeptide comprises a sequence selected from SEQ ID NOS: 14-18.

Embodiment 43 provides the method of any one of embodiments 34-42, wherein the modified FGF-1 polypeptide comprises a sequence selected from SEQ ID NOS: 24-28.

Embodiment 44 provides the method of any one of embodiments 34-43, wherein the modified FGF-1 polypeptide comprises a sequence selected from SEQ ID NOS: 93-117.

Embodiment 45 provides the method of any one of embodiments 34-44, wherein the modified FGF-1 polypeptide further comprises a methionine residue N-terminal to the extension peptide.

Embodiment 46 provides the method of any one of embodiments 34-45, wherein the modified FGF-1 polypeptide comprises a sequence selected from SEQ ID NOS: 118-141 and 207.

Embodiment 47 provides the method of any one of embodiments 34-46, wherein the modified FGF-1 polypeptide is expressed in a form that comprises 136 amino acids.

Embodiment 48 provides the method of any one of embodiments 34-46, wherein the modified FGF-1 polypeptide comprises at least 141 amino acids in its mature form.

Embodiment 49 provides the method of any one of embodiments 34-48, wherein the modified FGF-1 polypeptide further comprises a truncation of one or more of the first five residues of SEQ ID NO: 1.

Embodiment 50 provides the method of any one of embodiments 34-49, wherein the modified FGF-1 polypeptide comprises a sequence selected from SEQ ID NOS: 146-149, and 174-204.

Embodiment 51 provides the method of any one of embodiments 34-50, wherein the modified FGF-1 polypeptide further comprises an extension peptide comprising one or more amino acid residues of SEQ ID NO: 3.

Embodiment 52 provides the method of any one of embodiments 34-51, wherein the modified FGF-1 polypeptide comprises a sequence as set forth in SEQ ID NO: 2 SEQ ID NO: 205.

Embodiment 53 provides the method of embodiment 34, wherein the suitable cell is an E. coli cell, strain BL21, K12 HMS174, or W3110.

Embodiment 54 provides the method of embodiment 34, wherein the recombinant nucleic acid construct is pMKet_TTHX1114 comprising a T7 or tac promoter.

Embodiment 55 provides the method of embodiment 34, wherein the synthetic media comprises glycerol as carbon source, peptone and yeast.

Embodiment 56 provides the method of embodiment 34, wherein the. BL21 cells expressing pMKet_TTHX1114 were grown at 37° C. for 20 hours in presence of kanamycin.

Embodiment 57 provides the method of embodiment 34, wherein the recombinant nucleic acid construct comprises one or more modification for increasing yield of the modified FGF-1 polypeptide from the cell.

Embodiment 58 provides the method of embodiment 57, wherein the one or more modifications comprise sequence optimization for increased expression of the modified FGF-1 polypeptide in the cell.

Embodiment 59 provides the method of embodiment 57, wherein the one or more modifications comprise selecting a suitable promoter for increasing yield of the modified FGF-1 polypeptide from the cell.

Embodiment 60 provides the method of embodiments 34, further comprising growing the cell in adequate nutrient media for maximizing cell proliferation, wherein the adequate nutrient media comprises a carbon source, and wherein the carbon source is glucose or glycerol.

Embodiment 61 provides the method of embodiment 57, wherein the plasmid is pMKet, or a derivation or modification thereof.

Embodiment 62 provides the method of any one of the embodiments 34-61, further comprising introducing one or more modifications for maximizing the yield of the modified FGF-1 polypeptide from the cell, wherein the one or more modification processes are selected from: a modification within the recombinant nucleic acid encoding the modified FGF-1 polypeptide; a modification within the recombinant nucleic acid comprising one or more regulatory elements operably related to the recombinant nucleic acid encoding the modified FGF-1 polypeptide; a modification of the plasmid comprising the recombinant nucleic acid; a modification of the cell strain or selection of a cell strain for maximizing cell proliferation; and a modification of the cell growth media.

Embodiment 63 provides the method of embodiment 34, wherein introducing a recombinant nucleic acid comprises electroporating the recombinant nucleic acid in the cell.

Embodiment 64 provides the method of embodiment 34, wherein recovering the modified FGF-1 polypeptide from the cell comprises recovering the protein from periplasmic inclusion bodies of the cell.

Embodiment 65 provides the method of embodiment 64, wherein recovering comprises subjecting the inclusion bodies to solubilization in a denaturing buffer, and recovering the modified FGF-1 polypeptide.

Embodiment 66 provides the method of any one of the embodiments 65, wherein the denaturing buffer comprises urea or guanidine.

Embodiment 67 provides the method of embodiment 66, wherein the denaturing buffer further comprises 2 mM EDTA.

Embodiment 68 provides the method of any one of the embodiments 65-67, further comprising reducing the recovered modified FGF-1 polypeptide by adding DTT, further comprising removing DTT by diafiltration.

Embodiment 69 provides the method of any one of the embodiments 65-68, wherein the recovered modified FGF-1 polypeptide is subjected to refolding in a refolding buffer.

Embodiment 70 provides the method of embodiment 69, wherein the refolding buffer comprises L-arginine.

Embodiment 71 provides the method of any one of the embodiments 69 or 70, wherein the refolding buffer comprises 5 mM Cysteine, or 2 mM Cystine or both.

Embodiment 72 provides the method of any one of the embodiments 69-71, wherein the FGF-1 is captured by hydrophobic interaction column (HIC) with heparin.

Embodiment 73 provides the method of embodiment 34, wherein the recovering a therapeutically effective recombinant mutant hFGF1 protein comprises purifying the protein, wherein purifying comprises one or more of: liquid chromatography, hydrophobic interaction chromatography, affinity chromatography, ultracentrifugation, transverse flow filtration, and diafiltration.

Embodiment 75 provides a pharmaceutical composition comprising the modified FGF-1 polypeptide, produced by a method of any one of the embodiments 34-73, a lyophilized powder fraction thereof, or a liquid formulation thereof.

Embodiment 76 provides a method of treating a subject having a disease, a disorder, or a condition selected from a list consisting of: Fuch's dystrophy, bullous keratopathy, herpetic keratopathy, congenital hereditary endothelial dystrophy 1, congenital hereditary endothelial dystrophy 2, posterior polymorphous corneal dystrophy, a dry eye syndrome, keratoconus, lattice corneal dystrophy, granular corneal dystrophy, macular corneal dystrophy, Schnyder crystalline corneal dystrophy, congenital stromal corneal dystrophy, fleck corneal dystrophy, corneal injury, ocular injury, chemical injury, vesicant injury, stromal injury and mustard gas keratopathy, the method comprising administering to the subject in need thereof, a suitable dose of: (i) the injectable formulation of any one of the embodiments 1-33, or (ii) the pharmaceutical composition of embodiment 74.

Embodiment 77 provides a kit, comprising an injectable formulation of FGF-1.

Embodiment 78 provides the kit of embodiment 78, comprising a dropper bottle, wherein the dropper bottle is enabled to provide at least on dose of modified FGF-1 in the formulation of any one of the embodiments 1-33 or in the pharmaceutical composition of embodiment 74.

Embodiment 79 provides the kit of embodiment 77 or 78, wherein the dropper bottle further comprises a sterile filter.

Embodiment 80 provides the kit of any one of embodiments 77-79, wherein the container comprises the syringe.

Embodiment 81 provides the kit of embodiment 80, wherein the syringe comprises a material selected from the group consisting of tuberculin polypropylene and glass.

Embodiment 82 provides the kit of embodiment 81 or 82, wherein the syringe is prefilled with an injectable formulation according to any one of embodiments 1-33 or, a pharmaceutical composition according to embodiment 74.

Embodiment 83 provides the kit of any one of embodiments 80-82, further comprising an electronic control unit.

Embodiment 84 the kit of embodiment 83, wherein the electronic control unit enables control of administration of a volume of an injectable formulation according to any one of embodiments 1-33 or, a pharmaceutical composition according to embodiment 74, wherein the volume is from at least about 10 μL to about 100 μL.

TABLE OF
SEQUENCES

| SEQUENCE | No. |
|---|---|
| FNLPPGNYKKPKLLYCSNGGHFLRILPDGTVDGTRDRSDQHIQLQLSAESVGEVY | 1 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MFNLPPGNYKKPKLLYSSNGGHFLRILPDGTVDGTRDRSDQHIQLQLSAESVGEVYIKSTETGQYLCMD | 2 |
| TDGLLYGSQTPNEECLFLERLEENHYNTYISKKHAEKNWFVGLKKNGSVKRGPRTHYGQKAILFLPLPV | |
| SSD | |
| MAEGEITTFTALTEK | 3 |
| ALTEK | 4 |
| LTEK | 5 |
| TEK | 6 |
| EK | 7 |
| K | 8 |
| MALTEKFNLPPGNYKKPKLLYCSNGGHFLRILPDGTVDGTRDRSDQHIQLQLSAESVGEVY | 9 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKFNLPPGNYKKPKLLYCSNGGHFLRILPDGTVDGTRDRSDQHIQLQLSAESVGEVY | 10 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKFNLPPGNYKKPKLLYCSNGGHFLRILPDGTVDGTRDRSDQHIQLQLSAESVGEVY | 11 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKFNLPPGNYKKPKLLYCSNGGHFLRILPDGTVDGTRDRSDQHIQLQLSAESVGEVY | 12 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKFNLPPGNYKKPKLLYCSNGGHFLRILPDGTVDGTRDRSDQHIQLQLSAESVGEVY | 13 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKFNLPPGNYKKPKLLYSSNGGHFLRILPDGTVDGTRDRSDQHIQLQLSAESVGEVYIKSTETGQ | 14 |
| YLCMDTDGLLYGSQTPNEECLFLERLEENHYNTYISKKHAEKNWFVGLKKNGSVKRGPRTHYGQKAILF | |
| LPLPVSSD | |
| MLTEKFNLPPGNYKKPKLLYSSNGGHFLRILPDGTVDGTRDRSDQHIQLQLSAESVGEVYIKSTETGQY | 15 |
| LCMDTDGLLYGSQTPNEECLFLERLEENHYNTYISKKHAEKNWFVGLKKNGSVKRGPRTHYGQKAILFL | |
| PLPVSSD | |
| MTEKFNLPPGNYKKPKLLYSSNGGHFLRILPDGTVDGTRDRSDQHIQLQLSAESVGEVYIKSTETGQYL | 16 |
| CMDTDGLLYGSQTPNEECLFLERLEENHYNTYISKKHAEKNWFVGLKKNGSVKRGPRTHYGQKAILFLP | |
| LPVSSD | |
| MEKFNLPPGNYKKPKLLYSSNGGHFLRILPDGTVDGTRDRSDQHIQLQLSAESVGEVYIKSTETGQYLC | 17 |
| MDTDGLLYGSQTPNEECLFLERLEENHYNTYISKKHAEKNWFVGLKKNGSVKRGPRTHYGQKAILFLPL | |
| PVSSD | |
| MKFNLPPGNYKKPKLLYSSNGGHFLRILPDGTVDGTRDRSDQHIQLQLSAESVGEVYIKSTETGQYLCM | 18 |
| DTDGLLYGSQTPNEECLFLERLEENHYNTYISKKHAEKNWFVGLKKNGSVKRGPRTHYGQKAILFLPLP | |
| VSSD | |
| ALTEKFNLPPGNYKKPKLLYCSNGGHFLRILPDGTVDGTRDRSDQHIQLQLSAESVGEVY | 19 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 20 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 21 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 22 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 23 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKFNLPPGNYKKPKLLYSSNGGHFLRILPDGTVDGTRDRSDQHIQLQLSAESVGEVYIKSTETGQY | 24 |
| LCMDTDGLLYGSQTPNEECLFLERLEENHYNTYISKKHAEKNWFVGLKKNGSVKRGPRTHYGQKAILFL | |
| PLPVSSD | |
| LTEKFNLPPGNYKKPKLLYSSNGGHFLRILPDGTVDGTRDRSDQHIQLQLSAESVGEVYIKSTETGQYL | 25 |
| CMDTDGLLYGSQTPNEECLFLERLEENHYNTYISKKHAEKNWFVGLKKNGSVKRGPRTHYGQKAILFLP | |
| LPVSSD | |
| TEKFNLPPGNYKKPKLLYSSNGGHFLRILPDGTVDGTRDRSDQHIQLQLSAESVGEVYIKSTETGQYLC | 26 |
| MDTDGLLYGSQTPNEECLFLERLEENHYNTYISKKHAEKNWFVGLKKNGSVKRGPRTHYGQKAILFLPL | |
| PVSSD | |
| EKFNLPPGNYKKPKLLYSSNGGHFLRILPDGTVDGTRDRSDQHIQLQLSAESVGEVYIKSTETGQYLCM | 27 |
| DTDGLLYGSQTPNEECLFLERLEENHYNTYISKKHAEKNWFVGLKKNGSVKRGPRTHYGQKAILFLPLP | |
| VSSD | |
| KFNLPPGNYKKPKLLYSSNGGHFLRILPDGTVDGTRDRSDQHIQLQLSAESVGEVYIKSTETGQYLCMD | 28 |
| TDGLLYGSQTPNEECLFLERLEENHYNTYISKKHAEKNWFVGLKKNGSVKRGPRTHYGQKAILFLPLPV | |
| SSD | |
| NLPPGNYKKPKLLYCSNGGHFLRILPDGTVDGTRDRSDQHIQLQLSAESVGEVY | 29 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LPPGNYKKPKLLYCSNGGHFLRILPDGTVDGTRDRSDQHIQLQLSAESVGEVY | 30 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| PPGNYKKPKLLYCSNGGHFLRILPDGTVDGTRDRSDQHIQLQLSAESVGEVY | 31 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| PGNYKKPKLLYCSNGGHFLRILPDGTVDGTRDRSDQHIQLQLSAESVGEVY | 32 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| NLPPGNYKKPKLLYSSNGGHFLRILPDGTVDGTRDRSDQHIQLQLSAESVGEVYIKSTETGQYLCMDTD | 33 |
| GLLYGSQTPNEECLFLERLEENHYNTYISKKHAEKNWFVGLKKNGSVKRGPRTHYGQKAILFLPLPVSS | |
| D | |
| LPPGNYKKPKLLYSSNGGHFLRILPDGTVDGTRDRSDQHIQLQLSAESVGEVYIKSTETGQYLCMDTDG | 34 |
| LLYGSQTPNEECLFLERLEENHYNTYISKKHAEKNWFVGLKKNGSVKRGPRTHYGQKAILFLPLPVSSD | |
| PPGNYKKPKLLYSSNGGHFLRILPDGTVDGTRDRSDQHIQLQLSAESVGEVYIKSTETGQYLCMDTDGL | 35 |
| LYGSQTPNEECLFLERLEENHYNTYISKKHAEKNWFVGLKKNGSVKRGPRTHYGQKAILFLPLPVSSD | |
| PGNYKKPKLLYSSNGGHFLRILPDGTVDGTRDRSDQHIQLQLSAESVGEVYIKSTETGQYLCMDTDGLL | 36 |
| YGSQTPNEECLFLERLEENHYNTYISKKHAEKNWFVGLKKNGSVKRGPRTHYGQKAILFLPLPVSSD | |
| MNLPPGNYKKPKLLYCSNGGHFLRILPDGTVDGTRDRSDQHIQLQLSAESVGEVY | 37 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLPPGNYKKPKLLYCSNGGHFLRILPDGTVDGTRDRSDQHIQLQLSAESVGEVY | 38 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MPPGNYKKPKLLYCSNGGHFLRILPDGTVDGTRDRSDQHIQLQLSAESVGEVY | 39 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MPGNYKKPKLLYCSNGGHFLRILPDGTVDGTRDRSDQHIQLQLSAESVGEVY | 40 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MNLPPGNYKKPKLLYSSNGGHFLRILPDGTVDGTRDRSDQHIQLQLSAESVGEVYIKSTETGQYLCMDT | 41 |
| DGLLYGSQTPNEECLFLERLEENHYNTYISKKHAEKNWFVGLKKNGSVKRGPRTHYGQKAILFLPLPVS | |
| SD | |
| MLPPGNYKKPKLLYSSNGGHFLRILPDGTVDGTRDRSDQHIQLQLSAESVGEVYIKSTETGQYLCMDTD | 42 |
| GLLYGSQTPNEECLFLERLEENHYNTYISKKHAEKNWFVGLKKNGSVKRGPRTHYGQKAILFLPLPVSS | |
| D | |
| MPPGNYKKPKLLYSSNGGHFLRILPDGTVDGTRDRSDQHIQLQLSAESVGEVYIKSTETGQYLCMDTDG | 43 |
| LLYGSQTPNEECLFLERLEENHYNTYISKKHAEKNWFVGLKKNGSVKRGPRTHYGQKAILFLPLPVSSD | |
| MPGNYKKPKLLYSSNGGHFLRILPDGTVDGTRDRSDQHIQLQLSAESVGEVYIKSTETGQYLCMDTDGL | 44 |
| LYGSQTPNEECLFLERLEENHYNTYISKKHAEKNWFVGLKKNGSVKRGPRTHYGQKAILFLPLPVSSD | |
| MALTEKNLPPGNYKKPKLLYCSNGGHFLRILPDGTVDGTRDRSDQHIQLQLSAESVGEVY | 45 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKLPPGNYKKPKLLYSSNGGHFLRILPDGTVDGTRDRSDQHIQLQLSAESVGEVYIKSTETGQYL | 46 |
| CMDTDGLLYGSQTPNEECLFLERLEENHYNTYISKKHAEKNWFVGLKKNGSVKRGPRTHYGQKAILFLP | |
| LPVSSD | |
| MALTEKPPGGNYKKPKLLYCSNGGHFLRILPDGTVDGTRDRSDQHIQLQLSAESVGEVY | 47 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKPGNYKKPKLLYCSNGGHFLRILPDGTVDGTRDRSDQHIQLQLSAESVGEVY | 48 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKFNLPPGNYKKPKLLYCSNGGHFLRILPDGTVDGTRDRSDQHIQLQLSAESVGEVY | 49 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKNLPPGNYKKPKLLYCSNGGHFLRILPDGTVDGTRDRSDQHIQLQLSAESVGEVY | 50 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKLPPGNYKKPKLLYCSNGGHFLRILPDGTVDGTRDRSDQHIQLQLSAESVGEVY | 51 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKPPGNYKKPKLLYCSNGGHFLRILPDGTVDGTRDRSDQHIQLQLSAESVGEVY | 52 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKPGNYKKPKLLYCSNGGHFLRILPDGTVDGTRDRSDQHIQLQLSAESVGEVY | 53 |
| IKSTETGQYLAMDTDGLLYGSQTPNEECLFLERLEENHYNTYI SKKHAEK | |
| NWFVGLKKNGSCKRGPRTHYGQKAILFLPLPVSSD | |
| MTEKFNLPPGNYKKPKLLYCSNGGHFLRILPDGTVDGTRDRSDQHIQLQLSAESVGEVY | 54 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKNLPPGNYKKPKLLYCSNGGHFLRILPDGTVDGTRDRSDQHIQLQLSAESVGEVY | 55 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKLPPGNYKKPKLLYCSNGGHFLRILPDGTVDGTRDRSDQHIQLQLSAESVGEVY | 56 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKPPGNYKKPKLLYCSNGGHFLRILPDGTVDGTRDRSDQHIQLQLSAESVGEVY | 57 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKPGNYKKPKLLYCSNGGHFLRILPDGTVDGTRDRSDQHIQLQLSAESVGEVY | 58 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKFNLPPGNYKKPKLLYCSNGGHFLRILPDGTVDGTRDRSDQHIQLQLSAESVGEVY | 59 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKNLPPGNYKKPKLLYCSNGGHFLRILPDGTVDGTRDRSDQHIQLQLSAESVGEVY | 60 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKLPPGNYKKPKLLYCSNGGHFLRILPDGTVDGTRDRSDQHIQLQLSAESVGEVY | 61 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKPPGNYKKPKLLYCSNGGHFLRILPDGTVDGTRDRSDQHIQLQLSAESVGEVY | 62 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKPGNYKKPKLLYCSNGGHFLRILPDGTVDGTRDRSDQHIQLQLSAESVGEVY | 63 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKFNLPPGNYKKPKLLYCSNGGHFLRILPDGTVDGTRDRSDQHIQLQLSAESVGEVY | 64 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKNLPPGNYKKPKLLYCSNGGHFLRILPDGTVDGTRDRSDQHIQLQLSAESVGEVY | 65 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKLPPGNYKKPKLLYCSNGGHFLRILPDGTVDGTRDRSDQHIQLQLSAESVGEVY | 66 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKPPGNYKKPKLLYCSNGGHFLRILPDGTVDGTRDRSDQHIQLQLSAESVGEVY | 67 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKPGNYKKPKLLYCSNGGHFLRILPDGTVDGTRDRSDQHIQLQLSAESVGEVY | 68 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKNLPPGNYKKPKLLYCSNGGHFLRILPDGTVDGTRDRSDQHIQLQLSAESVGEVY | 69 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKLPPGNYKKPKLLYCSNGGHFLRILPDGTVDGTRDRSDQHIQLQLSAESVGEVY | 70 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKPPGNYKKPKLLYCSNGGHFLRILPDGTVDGTRDRSDQHIQLOLSAESVGEVY | 71 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKPGNYKKPKLLYCSNGGHFLRILPDGTVDGTRDRSDQHIQLQLSAESVGEVY | 72 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 73 |
| IKSTETGQYLAMDTDGLLYGSQTPNEECLFLERLEENHYNTYI SKKHAEK | |
| NWFVGLKKNGSCKRGPRTHYGQKAILFLPLPVSSD | |
| LTEKNLPPGNYKKPKLLYCSNGGHFLRILPDGTVDGTRDRSDQHIQLQLSAESVGEVY | 74 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKLPPGNYKKPKLLYCSNGGHFLRILPDGTVDGTRDRSDQHIQLQLSAESVGEVY | 75 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKPPGNYKKPKLLYCSNGGHFLRILPDGTVDGTRDRSDQHIQLQLSAESVGEVY | 76 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKPGNYKKPKLLYCSNGGHFLRILPDGTVDGTRDRSDQHIQLQLSAESVGEVY | 77 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 78 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKNLPPGNYKKPKLLYCSNGGHFLRILPDGTVDGTRDRSDQHIQLQLSAESVGEVY | 79 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKLPPGNYKKPKLLYCSNGGHFLRILPDGTVDGTRDRSDQHIQLQLSAESVGEVY | 80 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKPPGNYKKPKLLYCSNGGHFLRILPDGTVDGTRDRSDQHIQLQLSAESVGEVY | 81 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKPGNYKKPKLLYCSNGGHFLRILPDGTVDGTRDRSDQHIQLQLSAESVGEVY | 82 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 83 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKNLPPGNYKKPKLLYCSNGGHFLRILPDGTVDGTRDRSDQHIQLQLSAESVGEVY | 84 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKLPPGNYKKPKLLYCSNGGHFLRILPDGTVDGTRDRSDQHIQLQLSAESVGEVY | 85 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKPPGNYKKPKLLYCSNGGHFLRILPDGTVDGTRDRSDQHIQLQLSAESVGEVY | 86 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKPGNYKKPKLLYCSNGGHFLRILPDGTVDGTRDRSDQHIQLQLSAESVGEVY | 87 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 88 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KNLPPGNYKKPKLLYCSNGGHFLRILPDGTVDGTRDRSDQHIQLQLSAESVGEVY | 89 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KLPPGNYKKPKLLYCSNGGHFLRILPDGTVDGTRDRSDQHIQLQLSAESVGEVY | 90 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KPPGNYKKPKLLYCSNGGHFLRILPDGTVDGTRDRSDQHIQLQLSAESVGEVY | 91 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KPGNYKKPKLLYCSNGGHFLRILPDGTVDGTRDRSDQHIQLQLSAESVGEVY | 92 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKNLPPGNYKKPKLLYSSNGGHFLRILPDGTVDGTRDRSDQHIQLQLSAESVGEVYIKSTETGQYL | 93 |
| CMDTDGLLYGSQTPNEECLFLERLEENHYNTYISKKHAEKNWFVGLKKNGSVKRGPRTHYGQKAILFLP | |
| LPVSSD | |
| ALTEKLPPGNYKKPKLLYSSNGGHFLRILPDGTVDGTRDRSDQHIQLQLSAESVGEVYIKSTETGQYLC | 94 |
| MDTDGLLYGSQTPNEECLFLERLEENHYNTYISKKHAEKNWFVGLKKNGSVKRGPRTHYGQKAILFLPL | |
| PVSSD | |
| ALTEKPPGNYKKPKLLYSSNGGHFLRILPDGTVDGTRDRSDQHIQLQLSAESVGEVYIKSTETGQYLCM | 96 |
| DTDGLLYGSQTPNEECLFLERLEENHYNTYISKKHAEKNWFVGLKKNGSVKRGPRTHYGQKAILELPLP | |
| VSSD | |
| ALTEKPGNYKKPKLLYSSNGGHFLRILPDGTVDGTRDRSDQHIQLQLSAESVGEVYIKSTETGQYLCMD | 97 |
| TDGLLYGSQTPNEECLFLERLEENHYNTYISKKHAEKNWFVGLKKNGSVKRGPRTHYGQKAILFLPLPV | |
| SSD | |
| LTEKFNLPPGNYKKPKLLYSSNGGHFLRILPDGTVDGTRDRSDQHIQLQLSAESVGEVYIKSTETGQYL | 98 |
| CMDTDGLLYGSQTPNEECLFLERLEENHYNTYISKKHAEKNWFVGLKKNGSVKRGPRTHYGQKAILFLP | |
| LPVSSD | |
| LTEKNLPPGNYKKPKLLYSSNGGHFLRILPDGTVDGTRDRSDQHIQLQLSAESVGEVYIKSTETGQYLC | 99 |
| MDTDGLLYGSQTPNEECLFLERLEENHYNTYISKKHAEKNWFVGLKKNGSVKRGPRTHYGQKAILFLPL | |
| PVSSD | |
| LTEKLPPGNYKKPKLLYSSNGGHFLRILPDGTVDGTRDRSDQHIQLQLSAESVGEVYIKSTETGQYLCM | 100 |
| DTDGLLYGSQTPNEECLFLERLEENHYNTYISKKHAEKNWFVGLKKNGSVKRGPRTHYGQKAILFLPLP | |
| VSSD | |
| LTEKPPGNYKKPKLLYSSNGGHFLRILPDGTVDGTRDRSDQHIQLQLSAESVGEVYIKSTETGQYLCMD | 101 |
| TDGLLYGSQTPNEECLFLERLEENHYNTYISKKHAEKNWFVGLKKNGSVKRGPRTHYGQKAILFLPLPV | |
| SSD | |
| LTEKPGNYKKPKLLYSSNGGHFLRILPDGTVDGTRDRSDQHIQLQLSAESVGEVYIKSTETGQYLCMDT | 102 |
| DGLLYGSQTPNEECLFLERLEENHYNTYISKKHAEKNWFVGLKKNGSVKRGPRTHYGQKAILFLPLPVS | |
| SD | |
| TEKFNLPPGNYKKPKLLYSSNGGHFLRILPDGTVDGTRDRSDQHIQLQLSAESVGEVYIKSTETGQYLC | 103 |
| MDTDGLLYGSQTPNEECLFLERLEENHYNTYISKKHAEKNWFVGLKKNGSVKRGPRTHYGQKAILFLPL | |
| PVSSD | |
| TEKNLPPGNYKKPKLLYSSNGGHFLRILPDGTVDGTRDRSDQHIQLQLSAESVGEVYIKSTETGQYLCM | 104 |
| DTDGLLYGSQTPNEECLFLERLEENHYNTYISKKHAEKNWFVGLKKNGSVKRGPRTHYGQKAILFLPLP | |
| VSSD | |
| TEKLPPGNYKKPKLLYSSNGGHFLRILPDGTVDGTRDRSDQHIQLOLSAESVGEVYIKSTETGQYLCMD | 105 |
| TDGLLYGSQTPNEECLFLERLEENHYNTYISKKHAEKNWFVGLKKNGSVKRGPRTHYGQKAILFLPLPV | |
| SSD | |
| TEKPPGNYKKPKLLYSSNGGHFLRILPDGTVDGTRDRSDQHIQLOLSAESVGEVYIKSTETGQYLCMDT | 106 |
| DGLLYGSQTPNEECLFLERLEENHYNTYISKKHAEKNWFVGLKKNGSVKRGPRTHYGQKAILFLPLPVS | |
| SD | |
| TEKPGNYKKPKLLYSSNGGHFLRILPDGTVDGTRDRSDQHIQLQLSAESVGEVYIKSTETGQYLCMDTD | 107 |
| GLLYGSQTPNEECLFLERLEENHYNTYISKKHAEKNWFVGLKKNGSVKRGPRTHYGQKAILFLPLPVSS | |
| D | |
| EKFNLPPGNYKKPKLLYSSNGGHFLRILPDGTVDGTRDRSDQHIQLQLSAESVGEVYIKSTETGQYLCM | 108 |
| DTDGLLYGSQTPNEECLFLERLEENHYNTYISKKHAEKNWFVGLKKNGSVKRGPRTHYGQKAILFLPLP | |
| VSSD | |
| EKNLPPGNYKKPKLLYSSNGGHFLRILPDGTVDGTRDRSDQHIQLQLSAESVGEVYIKSTETGQYLCMD | 109 |
| TDGLLYGSQTPNEECLFLERLEENHYNTYISKKHAEKNWFVGLKKNGSVKRGPRTHYGQKAILFLPLPV | |
| SSD | |
| EKLPPGNYKKPKLLYSSNGGHFLRILPDGTVDGTRDRSDQHIQLQLSAESVGEVYIKSTETGQYLCMDT | 110 |
| DGLLYGSQTPNEECLFLERLEENHYNTYISKKHAEKNWFVGLKKNGSVKRGPRTHYGQKAILFLPLPVS | |
| SD | |
| EKPPGNYKKPKLLYSSNGGHFLRILPDGTVDGTRDRSDQHIQLQLSAESVGEVYIKSTETGQYLCMDTD | 111 |
| GLLYGSQTPNEECLFLERLEENHYNTYISKKHAEKNWFVGLKKNGSVKRGPRTHYGQKAILFLPLPVSS | |
| D | |
| EKPGNYKKPKLLYSSNGGHFLRILPDGTVDGTRDRSDQHIQLQLSAESVGEVYIKSTETGQYLCMDTDG | 112 |
| LLYGSQTPNEECLFLERLEENHYNTYISKKHAEKNWFVGLKKNGSVKRGPRTHYGQKAILFLPLPVSSD | |
| KFNLPPGNYKKPKLLYSSNGGHFLRILPDGTVDGTRDRSDQHIQLQLSAESVGEVYIKSTETGQYLCMD | 113 |
| TDGLLYGSQTPNEECLFLERLEENHYNTYISKKHAEKNWFVGLKKNGSVKRGPRTHYGQKAILFLPLPV | |
| SSD | |
| KNLPPGNYKKPKLLYSSNGGHFLRILPDGTVDGTRDRSDQHIQLQLSAESVGEVYIKSTETGQYLCMDT | 114 |
| DGLLYGSQTPNEECLFLERLEENHYNTYISKKHAEKNWFVGLKKNGSVKRGPRTHYGQKAILFLPLPVS | |
| SD | |
| KLPPGNYKKPKLLYSSNGGHFLRILPDGTVDGTRDRSDQHIQLQLSAESVGEVYIKSTETGQYLCMDTD | 115 |
| GLLYGSQTPNEECLFLERLEENHYNTYISKKHAEKNWFVGLKKNGSVKRGPRTHYGQKAILFLPLPVSS | |
| D | |
| KPPGNYKKPKLLYSSNGGHFLRILPDGTVDGTRDRSDQHIQLQLSAESVGEVYIKSTETGQYLCMDTDG | 116 |
| LLYGSQTPNEECLFLERLEENHYNTYISKKHAEKNWFVGLKKNGSVKRGPRTHYGQKAILFLPLPVSSD | |
| KPGNYKKPKLLYSSNGGHFLRILPDGTVDGTRDRSDQHIQLQLSAESVGEVYIKSTETGQYLCMDTDGL | 117 |
| LYGSQTPNEECLFLERLEENHYNTYISKKHAEKNWFVGLKKNGSVKRGPRTHYGQKAILFLPLPVSSD | |
| MALTEKFNLPPGNYKKPKLLYSSNGGHFLRILPDGTVDGTRDRSDQHIQLQLSAESVGEVYIKSTETGQ | 118 |
| YLCMDTDGLLYGSQTPNEECLFLERLEENHYNTYISKKHAEKNWFVGLKKNGSVKRGPRTHYGQKAILF | |
| LPLPVSSD | |
| MALTEKNLPPGNYKKPKLLYSSNGGHFLRILPDGTVDGTRDRSDQHIQLQLSAESVGEVYIKSTETGQY | 207 |
| LCMDTDGLLYGSQTPNEECLFLERLEENHYNTYISKKHAEKNWFVGLKKNGSVKRGPRTHYGQKAILFL | |
| PLPVSSD | |
| MALTEKLPPGNYKKPKLLYSSNGGHFLRILPDGTVDGTRDRSDQHIQLQLSAESVGEVYIKSTETGQYL | 119 |
| CMDTDGLLYGSQTPNEECLFLERLEENHYNTYISKKHAEKNWFVGLKKNGSVKRGPRTHYGQKAILFLP | |
| LPVSSD | |
| MALTEKPPGNYKKPKLLYSSNGGHFLRILPDGTVDGTRDRSDQHIQLQLSAESVGEVYIKSTETGQYLC | 120 |
| MDTDGLLYGSQTPNEECLFLERLEENHYNTYISKKHAEKNWFVGLKKNGSVKRGPRTHYGQKAILFLPL | |
| PVSSD | |
| MALTEKPGNYKKPKLLYSSNGGHFLRILPDGTVDGTRDRSDQHIQLQLSAESVGEVYIKSTETGQYLCM | 121 |
| DTDGLLYGSQTPNEECLFLERLEENHYNTYISKKHAEKNWFVGLKKNGSVKRGPRTHYGQKAILFLPLP | |
| VSSD | |
| MLTEKFNLPPGNYKKPKLLYSSNGGHFLRILPDGTVDGTRDRSDQHIQLQLSAESVGEVYIKSTETGQY | 122 |
| LCMDTDGLLYGSQTPNEECLFLERLEENHYNTYISKKHAEKNWFVGLKKNGSVKRGPRTHYGQKAILFL | |
| PLPVSSD | |
| MLTEKNLPPGNYKKPKLLYSSNGGHFLRILPDGTVDGTRDRSDQHIQLQLSAESVGEVYIKSTETGQYL | 123 |
| CMDTDGLLYGSQTPNEECLFLERLEENHYNTYISKKHAEKNWFVGLKKNGSVKRGPRTHYGQKAILFLP | |
| LPVSSD | |
| MLTEKLPPGNYKKPKLLYSSNGGHFLRILPDGTVDGTRDRSDQHIQLQLSAESVGEVYIKSTETGQYLC | 124 |
| MDTDGLLYGSQTPNEECLFLERLEENHYNTYISKKHAEKNWFVGLKKNGSVKRGPRTHYGQKAILFLPL | |
| PVSSD | |
| MLTEKPPGNYKKPKLLYSSNGGHFLRILPDGTVDGTRDRSDQHIQLQLSAESVGEVYIKSTETGQYLCM | 125 |
| DTDGLLYGSQTPNEECLFLERLEENHYNTYISKKHAEKNWFVGLKKNGSVKRGPRTHYGQKAILFLPLP | |
| VSSD | |
| MLTEKPGNYKKPKLLYSSNGGHFLRILPDGTVDGTRDRSDQHIQLQLSAESVGEVYIKSTETGQYLCMD | 126 |
| TDGLLYGSQTPNEECLFLERLEENHYNTYISKKHAEKNWFVGLKKNGSVKRGPRTHYGQKAILFLPLPV | |
| SSD | |
| MTEKFNLPPGNYKKPKLLYSSNGGHFLRILPDGTVDGTRDRSDQHIQLQLSAESVGEVYIKSTETGQYL | 127 |
| CMDTDGLLYGSQTPNEECLFLERLEENHYNTYISKKHAEKNWFVGLKKNGSVKRGPRTHYGQKAILFLP | |
| LPVSSD | |
| MTEKNLPPGNYKKPKLLYSSNGGHFLRILPDGTVDGTRDRSDQHIQLQLSAESVGEVYIKSTETGQYLC | 128 |
| MDTDGLLYGSQTPNEECLFLERLEENHYNTYISKKHAEKNWFVGLKKNGSVKRGPRTHYGQKAILFLPL | |
| PVSSD | |
| MTEKLPPGNYKKPKLLYSSNGGHFLRILPDGTVDGTRDRSDQHIQLQLSAESVGEVYIKSTETGQYLCM | 129 |
| DTDGLLYGSQTPNEECLFLERLEENHYNTYISKKHAEKNWFVGLKKNGSVKRGPRTHYGQKAILFLPLP | |
| VSSD | |
| MTEKPPGNYKKPKLLYSSNGGHFLRILPDGTVDGTRDRSDQHIQLQLSAESVGEVYIKSTETGQYLCMD | 130 |
| TDGLLYGSQTPNEECLFLERLEENHYNTYISKKHAEKNWFVGLKKNGSVKRGPRTHYGQKAILFLPLPV | |
| SSD | |
| MTEKPGNYKKPKLLYSSNGGHFLRILPDGTVDGTRDRSDQHIQLQLSAESVGEVYIKSTETGQYLCMDT | 131 |
| DGLLYGSQTPNEECLFLERLEENHYNTYISKKHAEKNWFVGLKKNGSVKRGPRTHYGQKAILFLPLPVS | |
| SD | |
| MEKFNLPPGNYKKPKLLYSSNGGHFLRILPDGTVDGTRDRSDQHIQLQLSAESVGEVYIKSTETGQYLC | 132 |
| MDTDGLLYGSQTPNEECLFLERLEENHYNTYISKKHAEKNWFVGLKKNGSVKRGPRTHYGQKAILFLPL | |
| PVSSD | |
| MEKNLPPGNYKKPKLLYSSNGGHFLRILPDGTVDGTRDRSDQHIQLQLSAESVGEVYIKSTETGQYLCM | 133 |
| DTDGLLYGSQTPNEECLFLERLEENHYNTYISKKHAEKNWFVGLKKNGSVKRGPRTHYGQKAILFLPLP | |
| VSSD | |
| MEKLPPGNYKKPKLLYSSNGGHFLRILPDGTVDGTRDRSDQHIQLQLSAESVGEVYIKSTETGQYLCMD | 134 |
| TDGLLYGSQTPNEECLFLERLEENHYNTYISKKHAEKNWFVGLKKNGSVKRGPRTHYGQKAILFLPLPV | |
| SSD | |
| MEKPPGNYKKPKLLYSSNGGHFLRILPDGTVDGTRDRSDQHIQLQLSAESVGEVYIKSTETGQYLCMDT | 135 |
| DGLLYGSQTPNEECLFLERLEENHYNTYISKKHAEKNWFVGLKKNGSVKRGPRTHYGQKAILFLPLPVS | |
| SD | |
| MEKPGNYKKPKLLYSSNGGHFLRILPDGTVDGTRDRSDQHIQLQLSAESVGEVYIKSTETGQYLCMDTD | 136 |
| GLLYGSQTPNEECLFLERLEENHYNTYISKKHAEKNWFVGLKKNGSVKRGPRTHYGQKAILFLPLPVSS | |
| D | |
| MKFNLPPGNYKKPKLLYSSNGGHFLRILPDGTVDGTRDRSDQHIQLQLSAESVGEVYIKSTETGQYLCM | 137 |
| DTDGLLYGSQTPNEECLFLERLEENHYNTYISKKHAEKNWFVGLKKNGSVKRGPRTHYGQKAILFLPLP | |
| VSSD | |
| MKNLPPGNYKKPKLLYSSNGGHFLRILPDGTVDGTRDRSDQHIQLQLSAESVGEVYIKSTETGQYLCMD | 138 |
| TDGLLYGSQTPNEECLFLERLEENHYNTYISKKHAEKNWFVGLKKNGSVKRGPRTHYGQKAILFLPLPV | |
| SSD | |
| MKLPPGNYKKPKLLYSSNGGHFLRILPDGTVDGTRDRSDQHIQLQLSAESVGEVYIKSTETGQYLCMDT | 139 |
| DGLLYGSQTPNEECLFLERLEENHYNTYISKKHAEKNWFVGLKKNGSVKRGPRTHYGQKAILFLPLPVS | |
| SD | |
| MKPPGNYKKPKLLYSSNGGHFLRILPDGTVDGTRDRSDQHIQLQLSAESVGEVYIKSTETGQYLCMDTD | 140 |
| GLLYGSQTPNEECLFLERLEENHYNTYISKKHAEKNWFVGLKKNGSVKRGPRTHYGQKAILFLPLPVSS | |
| D | |
| MKPGNYKKPKLLYSSNGGHFLRILPDGTVDGTRDRSDQHIQLQLSAESVGEVYIKSTETGQYLCMDTDG | 141 |
| LLYGSQTPNEECLFLERLEENHYNTYISKKHAEKNWFVGLKKNGSVKRGPRTHYGQKAILFLPLPVSSD | |
| FNLPPGNYKKPKLLYCSNGGHFLRILPDGTVDGTRDRSDQHIQLQLSAESVGEVY | 142 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| NLPPGNYKKPKLLYCSNGGHFLRILPDGTVDGTRDRSDQHIQLQLSAESVGEVY | 143 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| PPGNYKKPKLLYCSNGGHFLRILPDGTVDGTRDRSDQHIQLQLSAESVGEVY | 144 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| PGNYKKPKLLYCSNGGHFLRILPDGTVDGTRDRSDQHIQLQLSAESVGEVY | 145 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| FNLPPGNYKKPKLLYSSNGGHFLRILPDGTVDGTRDRSDQHIQLQLSAESVGEVYIKSTETGQYLCXDT | 146 |
| DGLLYGSQTPNEECLFLERLEENHYNTYISKKHAEKNWFVGLKKNGSVKRGPRTHYGQKAILFLPLPVS | |
| SD | |
| NLPPGNYKKPKLLYSSNGGHFLRILPDGTVDGTRDRSDQHIQLQLSAESVGEVYIKSTETGQYLCXDTD | 147 |
| GLLYGSQTPNEECLFLERLEENHYNTYISKKHAEKNWFVGLKKNGSVKRGPRTHYGQKAILFLPLPVSS | |
| D | |
| PPGNYKKPKLLYSSNGGHFLRILPDGTVDGTRDRSDQHIQLQLSAESVGEVYIKSTETGQYLCXDTDGL | 148 |
| LYGSQTPNEECLFLERLEENHYNTYISKKHAEKNWFVGLKKNGSVKRGPRTHYGQKAILFLPLPVSSD | |
| PGNYKKPKLLYSSNGGHFLRILPDGTVDGTRDRSDQHIQLQLSAESVGEVYIKSTETGQYLCXDTDGLL | 149 |
| YGSQTPNEECLFLERLEENHYNTYISKKHAEKNWFVGLKKNGSVKRGPRTHYGQKAILFLPLPVSSD | |
| ALTEKNLPPGNYKKPKLLYCSNGGHFLRILPDGTVDGTRDRSDQHIQLQLSAESVGEVY | 150 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| ALTEKLPPGNYKKPKLLYSSNGGHFLRILPDGTVDGTRDRSDQHIQLQLSAESVGEVYIKSTETGQYLC | 151 |
| XDTDGLLYGSQTPNEECLFLERLEENHYNTYISKKHAEKNWFVGLKKNGSVKRGPRTHYGQKAILFLPL | |
| PVSSD | |
| ALTEKPPGGNYKKPKLLYCSNGGHFLRILPDGTVDGTRDRSDQHIQLQLSAESVGEVY | 152 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| ALTEKPGNYKKPKLLYCSNGGHFLRILPDGTVDGTRDRSDQHIQLQLSAESVGEVY | 153 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 154 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKNLPPGNYKKPKLLYCSNGGHFLRILPDGTVDGTRDRSDQHIQLQLSAESVGEVY | 155 |
| IKSTETGQYLAXDTDGLLYGSQTPNEECLFLERLEENHYNTYI SKKHAEK | |
| NWFVGLKKNGSCKRGPRTHYGQKAILFLPLPVSSD | |
| LTEKLPPGNYKKPKLLYCSNGGHFLRILPDGTVDGTRDRSDQHIQLQLSAESVGEVY | 156 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKPPGNYKKPKLLYCSNGGHFLRILPDGTVDGTRDRSDQHIQLQLSAESVGEVY | 157 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKPGNYKKPKLLYCSNGGHFLRILPDGTVDGTRDRSDQHIQLQLSAESVGEVY | 158 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 159 |
| IKSTETGQYLAXDTDGLLYGSQTPNEECLFLERLEENHYNTYI SKKHAEK | |
| NWFVGLKKNGSCKRGPRTHYGQKAILFLPLPVSSD | |
| TEKNLPPGNYKKPKLLYCSNGGHFLRILPDGTVDGTRDRSDQHIQLQLSAESVGEVY | 160 |
| IKSTETGQYLAXDTDGLLYGSQTPNEECLFLERLEENHYNTYI SKKHAEK | |
| NWFVGLKKNGSCKRGPRTHYGQKAILFLPLPVSSD | |
| TEKLPPGNYKKPKLLYCSNGGHFLRILPDGTVDGTRDRSDQHIQLQLSAESVGEVY | 161 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKPPGNYKKPKLLYCSNGGHFLRILPDGTVDGTRDRSDQHIQLQLSAESVGEVY | 162 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKPGNYKKPKLLYCSNGGHFLRILPDGTVDGTRDRSDQHIQLQLSAESVGEVY | 163 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 164 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKNLPPGNYKKPKLLYCSNGGHFLRILPDGTVDGTRDRSDQHIQLQLSAESVGEVY | 165 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKLPPGNYKKPKLLYCSNGGHFLRILPDGTVDGTRDRSDQHIQLQLSAESVGEVY | 166 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKPPGNYKKPKLLYCSNGGHFLRILPDGTVDGTRDRSDQHIQLQLSAESVGEVY | 167 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKPGNYKKPKLLYCSNGGHFLRILPDGTVDGTRDRSDQHIQLQLSAESVGEVY | 168 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 169 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KNLPPGNYKKPKLLYCSNGGHFLRILPDGTVDGTRDRSDQHIQLQLSAESVGEVY | 170 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KLPPGNYKKPKLLYCSNGGHFLRILPDGTVDGTRDRSDQHIQLQLSAESVGEVY | 171 |
| IKSTETGQYLAXDTDGLLYGSQTPNEECLFLERLEENHYNTYI SKKHAEK | |
| NWFVGLKKNGSCKRGPRTHYGQKAILFLPLPVSSD | |
| KPPGNYKKPKLLYCSNGGHFLRILPDGTVDGTRDRSDQHIQLQLSAESVGEVY | 172 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KPGNYKKPKLLYCSNGGHFLRILPDGTVDGTRDRSDQHIQLQLSAESVGEVY | 173 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| FNLPPGNYKKPKLLYSSNGGHFLRILPDGTVDGTRDRSDQHIQLQLSAESVGEVYIKSTETGQYLCXDT | 174 |
| DGLLYGSQTPNEECLFLERLEENHYNTYISKKHAEKNWFVGLKKNGSVKRGPRTHYGQKAILFLPLPVS | |
| SD | |
| ALTEKFNLPPGNYKKPKLLYSSNGGHFLRILPDGTVDGTRDRSDQHIQLQLSAESVGEVYIKSTETGQY | 175 |
| LCXDTDGLLYGSQTPNEECLFLERLEENHYNTYISKKHAEKNWFVGLKKNGSVKRGPRTHYGQKAILFL | |
| PLPVSSD | |
| LTEKFNLPPGNYKKPKLLYSSNGGHFLRILPDGTVDGTRDRSDQHIQLQLSAESVGEVYIKSTETGQYL | 176 |
| CXDTDGLLYGSQTPNEECLFLERLEENHYNTYISKKHAEKNWFVGLKKNGSVKRGPRTHYGQKAILFLP | |
| LPVSSD | |
| TEKFNLPPGNYKKPKLLYSSNGGHFLRILPDGTVDGTRDRSDQHIQLQLSAESVGEVYIKSTETGQYLC | 177 |
| XDTDGLLYGSQTPNEECLFLERLEENHYNTYISKKHAEKNWFVGLKKNGSVKRGPRTHYGQKAILFLPL | |
| PVSSD | |
| EKFNLPPGNYKKPKLLYSSNGGHFLRILPDGTVDGTRDRSDQHIQLQLSAESVGEVYIKSTETGQYLCX | 178 |
| DTDGLLYGSQTPNEECLFLERLEENHYNTYISKKHAEKNWFVGLKKNGSVKRGPRTHYGQKAILFLPLP | |
| VSSD | |
| KFNLPPGNYKKPKLLYSSNGGHFLRILPDGTVDGTRDRSDQHIQLQLSAESVGEVYIKSTETGQYLCXD | 179 |
| TDGLLYGSQTPNEECLFLERLEENHYNTYISKKHAEKNWFVGLKKNGSVKRGPRTHYGQKAILFLPLPV | |
| SSD | |
| KFNLPPGNYKKPKLLYSSNGGHFLRILPDGTVDGTRDRSDQHIQLQLSAESVGEVYIKSTETGQYLCXD | 180 |
| TDGLLYGSQTPNEECLFLERLEENHYNTYISKKHAEKNWFVGLKKNGSVKRGPRTHYGQKAILFLPLPV | |
| SSD | |
| ALTEKNLPPGNYKKPKLLYSSNGGHFLRILPDGTVDGTRDRSDQHIQLQLSAESVGEVYIKSTETGQYL | 181 |
| CXDTDGLLYGSQTPNEECLFLERLEENHYNTYISKKHAEKNWFVGLKKNGSVKRGPRTHYGQKAILFLP | |
| LPVSSD | |
| ALTEKLPPGNYKKPKLLYSSNGGHFLRILPDGTVDGTRDRSDQHIQLQLSAESVGEVYIKSTETGQYLC | 182 |
| XDTDGLLYGSQTPNEECLFLERLEENHYNTYISKKHAEKNWFVGLKKNGSVKRGPRTHYGQKAILFLPL | |
| PVSSD | |
| ALTEKPPGNYKKPKLLYSSNGGHFLRILPDGTVDGTRDRSDQHIQLQLSAESVGEVYIKSTETGQYLCX | 183 |
| DTDGLLYGSQTPNEECLFLERLEENHYNTYISKKHAEKNWFVGLKKNGSVKRGPRTHYGQKAILFLPLP | |
| VSSD | |
| ALTEKPGNYKKPKLLYSSNGGHFLRILPDGTVDGTRDRSDQHIQLQLSAESVGEVYIKSTETGQYLCXD | 184 |
| TDGLLYGSQTPNEECLFLERLEENHYNTYISKKHAEKNWFVGLKKNGSVKRGPRTHYGQKAILFLPLPV | |
| SSD | |
| LTEKFNLPPGNYKKPKLLYSSNGGHFLRILPDGTVDGTRDRSDOHIQLOLSAESVGEVYIKSTETGQYL | 185 |
| CXDTDGLLYGSQTPNEECLFLERLEENHYNTYISKKHAEKNWFVGLKKNGSVKRGPRTHYGQKAILFLP | |
| LPVSSD | |
| LTEKNLPPGNYKKPKLLYSSNGGHFLRILPDGTVDGTRDRSDQHIQLQLSAESVGEVYIKSTETGQYLC | 186 |
| XDTDGLLYGSQTPNEECLFLERLEENHYNTYISKKHAEKNWFVGLKKNGSVKRGPRTHYGQKAILFLPL | |
| PVSSD | |
| LTEKLPPGNYKKPKLLYSSNGGHFLRILPDGTVDGTRDRSDQHIQLQLSAESVGEVYIKSTETGQYLCX | 187 |
| DTDGLLYGSQTPNEECLFLERLEENHYNTYISKKHAEKNWFVGLKKNGSVKRGPRTHYGQKAILFLPLP | |
| VSSD | |
| LTEKPPGNYKKPKLLYSSNGGHFLRILPDGTVDGTRDRSDQHIQLQLSAESVGEVYIKSTETGQYLCXD | 188 |
| TDGLLYGSQTPNEECLFLERLEENHYNTYISKKHAEKNWFVGLKKNGSVKRGPRTHYGQKAILFLPLPV | |
| SSD | |
| LTEKPGNYKKPKLLYSSNGGHFLRILPDGTVDGTRDRSDQHIQLQLSAESVGEVYIKSTETGQYLCXDT | 189 |
| DGLLYGSQTPNEECLFLERLEENHYNTYISKKHAEKNWFVGLKKNGSVKRGPRTHYGQKAILFLPLPVS | |
| SD | |
| TEKFNLPPGNYKKPKLLYSSNGGHFLRILPDGTVDGTRDRSDQHIQLQLSAESVGEVYIKSTETGQYLC | 190 |
| XDTDGLLYGSQTPNEECLFLERLEENHYNTYISKKHAEKNWFVGLKKNGSVKRGPRTHYGQKAILFLPL | |
| PVSSD | |
| TEKNLPPGNYKKPKLLYSSNGGHFLRILPDGTVDGTRDRSDQHIQLQLSAESVGEVYIKSTETGQYLCX | 191 |
| DTDGLLYGSQTPNEECLFLERLEENHYNTYISKKHAEKNWFVGLKKNGSVKRGPRTHYGQKAILFLPLP | |
| VSSD | |
| TEKLPPGNYKKPKLLYSSNGGHFLRILPDGTVDGTRDRSDQHIQLQLSAESVGEVYIKSTETGQYLCXD | 192 |
| TDGLLYGSQTPNEECLFLERLEENHYNTYISKKHAEKNWFVGLKKNGSVKRGPRTHYGQKAILFLPLPV | |
| SSD | |
| TEKPPGNYKKPKLLYSSNGGHFLRILPDGTVDGTRDRSDQHIQLQLSAESVGEVYIKSTETGQYLCXDT | 193 |
| DGLLYGSQTPNEECLFLERLEENHYNTYISKKHAEKNWFVGLKKNGSVKRGPRTHYGQKAILFLPLPVS | |
| SD | |
| TEKPGNYKKPKLLYSSNGGHFLRILPDGTVDGTRDRSDQHIQLQLSAESVGEVYIKSTETGQYLCXDTD | 194 |
| GLLYGSQTPNEECLFLERLEENHYNTYISKKHAEKNWFVGLKKNGSVKRGPRTHYGQKAILFLPLPVSS | |
| D | |
| EKFNLPPGNYKKPKLLYSSNGGHFLRILPDGTVDGTRDRSDQHIQLQLSAESVGEVYIKSTETGQYLCX | 195 |
| DTDGLLYGSQTPNEECLFLERLEENHYNTYISKKHAEKNWFVGLKKNGSVKRGPRTHYGQKAILFLPLP | |
| VSSD | |
| EKNLPPGNYKKPKLLYSSNGGHFLRILPDGTVDGTRDRSDQHIQLQLSAESVGEVYIKSTETGQYLCXD | 196 |
| TDGLLYGSQTPNEECLFLERLEENHYNTYISKKHAEKNWFVGLKKNGSVKRGPRTHYGQKAILFLPLPV | |
| SSD | |
| EKLPPGNYKKPKLLYSSNGGHFLRILPDGTVDGTRDRSDQHIQLQLSAESVGEVYIKSTETGQYLCXDT | 197 |
| DGLLYGSQTPNEECLFLERLEENHYNTYISKKHAEKNWFVGLKKNGSVKRGPRTHYGQKAILFLPLPVS | |
| SD | |
| EKPPGNYKKPKLLYSSNGGHFLRILPDGTVDGTRDRSDQHIQLQLSAESVGEVYIKSTETGQYLCXDTD | 198 |
| GLLYGSQTPNEECLFLERLEENHYNTYISKKHAEKNWFVGLKKNGSVKRGPRTHYGQKAILFLPLPVSS | |
| D | |
| EKPGNYKKPKLLYSSNGGHFLRILPDGTVDGTRDRSDQHIQLQLSAESVGEVYIKSTETGQYLCXDTDG | 199 |
| LLYGSQTPNEECLFLERLEENHYNTYISKKHAEKNWFVGLKKNGSVKRGPRTHYGQKAILFLPLPVSSD | |
| KFNLPPGNYKKPKLLYSSNGGHFLRILPDGTVDGTRDRSDQHIQLQLSAESVGEVYIKSTETGQYLCXD | 200 |
| TDGLLYGSQTPNEECLFLERLEENHYNTYISKKHAEKNWFVGLKKNGSVKRGPRTHYGQKAILFLPLPV | |
| SSD | |
| KNLPPGNYKKPKLLYSSNGGHFLRILPDGTVDGTRDRSDQHIQLQLSAESVGEVYIKSTETGQYLCXDT | 201 |
| DGLLYGSQTPNEECLFLERLEENHYNTYISKKHAEKNWFVGLKKNGSVKRGPRTHYGQKAILFLPLPVS | |
| SD | |
| KLPPGNYKKPKLLYSSNGGHFLRILPDGTVDGTRDRSDQHIQLQLSAESVGEVYIKSTETGQYLCXDTD | 202 |
| GLLYGSQTPNEECLFLERLEENHYNTYISKKHAEKNWFVGLKKNGSVKRGPRTHYGQKAILFLPLPVSS | |
| D | |
| KPPGNYKKPKLLYSSNGGHFLRILPDGTVDGTRDRSDQHIQLQLSAESVGEVYIKSTETGQYLCXDTDG | 203 |
| LLYGSQTPNEECLFLERLEENHYNTYISKKHAEKNWFVGLKKNGSVKRGPRTHYGQKAILFLPLPVSSD | |
| KPGNYKKPKLLYSSNGGHFLRILPDGTVDGTRDRSDQHIQLQLSAESVGEVYIKSTETGQYLCXDTDGL | 204 |
| LYGSQTPNEECLFLERLEENHYNTYISKKHAEKNWFVGLKKNGSVKRGPRTHYGQKAILFLPLPVSSD | |
| FNLPPGNYKKPVLLYCSNGGHFLRILPDGTVDGTRDRSDQHIQLQLSAESVGEVY | 205 |
| IKSTETGQYLAMDTDGLLYGSQTPNEECLFLERLEENHYNTYISKKHAEK | |
| NWFVGLKKNGSVKRGPRTHYGQKAILFLVLPVSSD | |
| FNLPPGNYKKPKLLYSSNGGHFLRILPDGTVDGTRDRSDQHIQLQLSAESVGEVYIKSTETGQYLCMDT | 206 |
| DGLLYGSQTPNEECLFLERLEENHYNTYISKKHAEKNWFVGLKKNGSVKRGPRTHYGQKAILFLPLPVS | |
| SD | |

1. A pharmaceutical formulation comprising:
a modified fibroblast growth factor 1 (FGF-1) polypeptide;
citrate or histidine at a concentration of about 0.1 mM to about 20 mM;
a surfactant at a concentration of about 0.01% (w/v) to about 10% (w/v); and
a tonicity modifying agent at a concentration of about 1% (w/v) to about 10% (w/v) or from about 50 mM to about 200 mM,
wherein the modified FGF-1 polypeptide comprises:
a methionine residue at the amino-terminus of the modified FGF-1 polypeptide;
an amino acid sequence that is at least 90% identical to the wild-type FGF-1 amino acid sequence of SEQ ID NO: 1; and
a Cys16Ser mutation, an Ala66Cys mutation, and a Cys117Val mutation with reference to SEQ ID NO: 1.

2. The pharmaceutical formulation of claim 1, comprising the histidine at a concentration of about 1 mM or about 10 mM.

3. The pharmaceutical formulation of claim 1, wherein the concentration of the surfactant is about 0.1% (w/v).

4. The pharmaceutical formulation of claim 1, wherein the surfactant comprises a polysorbate.

5. The pharmaceutical formulation of claim 4, wherein the polysorbate comprises polysorbate 20 or polysorbate 80.

6. The pharmaceutical formulation of claim 4, wherein the polysorbate comprises polysorbate-80.

7. The pharmaceutical formulation of claim 1, wherein the tonicity modifying agent comprises sorbitol at a concentration of about 5% (w/v).

8. The pharmaceutical formulation of claim 1, further comprising a pH of about 4.5 to about 6.5.

9. The pharmaceutical formulation of claim 8, further comprising a pH of about 5.8.

10. The pharmaceutical formulation of claim 1, wherein the concentration of the modified FGF-1 polypeptide is from about 0.0005 μg/mL to about 200 μg/mL.

11. The pharmaceutical formulation of claim 10, wherein the concentration of the modified FGF-1 polypeptide is about 100 μg/mL.

12. The pharmaceutical formulation of claim 1, wherein the modified FGF-1 polypeptide is stable for at least about 28 days when the pharmaceutical formulation is stored at room temperature, as measured by any one of: (i) lack of visible particulates by visual inspection, (ii) a peak area less than about 5% for high molecular weight species in an SE-HPLC assay, and (iii) no significant change in pH of the pharmaceutical formulation.

13. The pharmaceutical formulation of claim 12, wherein the modified FGF-1 polypeptide is stable for at least about 50 days when the pharmaceutical formulation is stored at room temperature.

14. The pharmaceutical formulation of claim 13, wherein the modified FGF-1 polypeptide is stable for at least about 59 days when the pharmaceutical formulation is stored at room temperature.

15. The pharmaceutical formulation of claim 1, wherein the pharmaceutical formulation is suitable for topical application, intraocular injection, or periocular injection.

16. The pharmaceutical formulation of claim 1, wherein the pharmaceutical formulation is an injectable formulation for intraocular delivery.

17. The pharmaceutical formulation of claim 1, wherein the pharmaceutical formulation is an injectable formulation for intravitreal delivery.

18.-38. (canceled)

39. The pharmaceutical formulation of claim 1, further comprising the histidine at a concentration of about 10 mM, polysorbate 80 at a concentration of about 0.1% (w/v), sorbitol at a concentration of about 5% (w/v), and a pH of about 5.8.

40. The pharmaceutical formulation of claim 12, wherein the pH of the pharmaceutical formulation is stable for at least about 59 days when the pharmaceutical formulation is stored at room temperature.

41. The pharmaceutical formulation of claim 1, wherein the pharmaceutical formulation is in the form of eye drop.

42. The pharmaceutical formulation of claim 1, wherein the modified FGF-1 polypeptide comprises an amino acid sequence that is at least about 90% identical to SEQ ID NO: 2.

43. The pharmaceutical formulation of claim 1, wherein the modified FGF-1 polypeptide comprises an amino acid sequence that is at least about 95% identical to SEQ ID NO: 2.

44. The pharmaceutical formulation of claim 1, wherein the modified FGF-1 polypeptide comprises the amino acid sequence of SEQ ID NO: 2.


SEQUENCE LISTING (207 sequences)

SEQ ID NO: 1 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 141 aa, synthetic construct
  REGION 1..141: note = Description of Artificial Sequence: Synthetic polypeptide
MFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 3 - protein, 15 aa, synthetic construct
  REGION 1..15: note = Description of Artificial Sequence: Synthetic peptide
MAEGEITTFTALTEK

SEQ ID NO: 4 - protein, 5 aa, synthetic construct
  REGION 1..5: note = Description of Artificial Sequence: Synthetic peptide
ALTEK

SEQ ID NO: 5 - protein, 4 aa, synthetic construct
  REGION 1..4: note = Description of Artificial Sequence: Synthetic peptide
LTEK

SEQ ID NO: 6-8 - skipped (3 entries)

SEQ ID NO: 9 - protein, 146 aa, synthetic construct
  REGION 1..146: note = Description of Artificial Sequence: Synthetic polypeptide
MALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 10 - protein, 145 aa, synthetic construct
  REGION 1..145: note = Description of Artificial Sequence: Synthetic polypeptide
MLTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - protein, 144 aa, synthetic construct
  REGION 1..144: note = Description of Artificial Sequence: Synthetic polypeptide
MTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 12 - protein, 143 aa, synthetic construct
  REGION 1..143: note = Description of Artificial Sequence: Synthetic polypeptide
MEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 13 - protein, 142 aa, synthetic construct
  REGION 1..142: note = Description of Artificial Sequence: Synthetic polypeptide
MKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 14 - protein, 146 aa, synthetic construct
  REGION 1..146: note = Description of Artificial Sequence: Synthetic polypeptide
MALTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 15 - protein, 145 aa, synthetic construct
  REGION 1..145: note = Description of Artificial Sequence: Synthetic polypeptide
MLTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 16 - protein, 144 aa, synthetic construct
  REGION 1..144: note = Description of Artificial Sequence: Synthetic polypeptide
MTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 17 - protein, 143 aa, synthetic construct
  REGION 1..143: note = Description of Artificial Sequence: Synthetic polypeptide
MEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 18 - protein, 142 aa, synthetic construct
  REGION 1..142: note = Description of Artificial Sequence: Synthetic polypeptide
MKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 19 - protein, 145 aa, synthetic construct
  REGION 1..145: note = Description of Artificial Sequence: Synthetic polypeptide
ALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 20 - protein, 144 aa, synthetic construct
  REGION 1..144: note = Description of Artificial Sequence: Synthetic polypeptide
LTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 21 - protein, 143 aa, synthetic construct
  REGION 1..143: note = Description of Artificial Sequence: Synthetic polypeptide
TEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 22 - protein, 142 aa, synthetic construct
  REGION 1..142: note = Description of Artificial Sequence: Synthetic polypeptide
EKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 23 - protein, 141 aa, synthetic construct
  REGION 1..141: note = Description of Artificial Sequence: Synthetic polypeptide
KFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 24 - protein, 145 aa, synthetic construct
  REGION 1..145: note = Description of Artificial Sequence: Synthetic polypeptide
ALTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 25 - protein, 144 aa, synthetic construct
  REGION 1..144: note = Description of Artificial Sequence: Synthetic polypeptide
LTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 26 - protein, 143 aa, synthetic construct
  REGION 1..143: note = Description of Artificial Sequence: Synthetic polypeptide
TEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 27 - protein, 142 aa, synthetic construct
  REGION 1..142: note = Description of Artificial Sequence: Synthetic polypeptide
EKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 28 - protein, 141 aa, synthetic construct
  REGION 1..141: note = Description of Artificial Sequence: Synthetic polypeptide
KFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 29 - protein, 139 aa, synthetic construct
  REGION 1..139: note = Description of Artificial Sequence: Synthetic polypeptide
NLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 30 - protein, 138 aa, synthetic construct
  REGION 1..138: note = Description of Artificial Sequence: Synthetic polypeptide
LPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 31 - protein, 137 aa, synthetic construct
  REGION 1..137: note = Description of Artificial Sequence: Synthetic polypeptide
PPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 32 - protein, 136 aa, synthetic construct
  REGION 1..136: note = Description of Artificial Sequence: Synthetic polypeptide
PGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 33 - protein, 139 aa, synthetic construct
  REGION 1..139: note = Description of Artificial Sequence: Synthetic polypeptide
NLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 34 - protein, 138 aa, synthetic construct
  REGION 1..138: note = Description of Artificial Sequence: Synthetic polypeptide
LPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 35 - protein, 137 aa, synthetic construct
  REGION 1..137: note = Description of Artificial Sequence: Synthetic polypeptide
PPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 36 - protein, 136 aa, synthetic construct
  REGION 1..136: note = Description of Artificial Sequence: Synthetic polypeptide
PGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 37 - protein, 140 aa, synthetic construct
  REGION 1..140: note = Description of Artificial Sequence: Synthetic polypeptide
MNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 38 - protein, 139 aa, synthetic construct
  REGION 1..139: note = Description of Artificial Sequence: Synthetic polypeptide
MLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 39 - protein, 138 aa, synthetic construct
  REGION 1..138: note = Description of Artificial Sequence: Synthetic polypeptide
MPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 40 - protein, 137 aa, synthetic construct
  REGION 1..137: note = Description of Artificial Sequence: Synthetic polypeptide
MPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 41 - protein, 140 aa, synthetic construct
  REGION 1..140: note = Description of Artificial Sequence: Synthetic polypeptide
MNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 42 - protein, 139 aa, synthetic construct
  REGION 1..139: note = Description of Artificial Sequence: Synthetic polypeptide
MLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 43 - protein, 138 aa, synthetic construct
  REGION 1..138: note = Description of Artificial Sequence: Synthetic polypeptide
MPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 44 - protein, 137 aa, synthetic construct
  REGION 1..137: note = Description of Artificial Sequence: Synthetic polypeptide
MPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 45 - protein, 145 aa, synthetic construct
  REGION 1..145: note = Description of Artificial Sequence: Synthetic polypeptide
MALTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 46 - protein, 144 aa, synthetic construct
  REGION 1..144: note = Description of Artificial Sequence: Synthetic polypeptide
MALTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 47 - protein, 144 aa, synthetic construct
  REGION 1..144: note = Description of Artificial Sequence: Synthetic polypeptide
MALTEKPPGGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 48 - protein, 142 aa, synthetic construct
  REGION 1..142: note = Description of Artificial Sequence: Synthetic polypeptide
MALTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 49 - protein, 145 aa, synthetic construct; identical to SEQ ID NO: 10
  REGION 1..145: note = Description of Artificial Sequence: Synthetic polypeptide

SEQ ID NO: 50 - protein, 144 aa, synthetic construct
  REGION 1..144: note = Description of Artificial Sequence: Synthetic polypeptide
MLTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 51 - protein, 143 aa, synthetic construct
  REGION 1..143: note = Description of Artificial Sequence: Synthetic polypeptide
MLTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 52 - protein, 142 aa, synthetic construct
  REGION 1..142: note = Description of Artificial Sequence: Synthetic polypeptide
MLTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 53 - protein, 141 aa, synthetic construct
  REGION 1..141: note = Description of Artificial Sequence: Synthetic polypeptide
MLTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 54 - protein, 144 aa, synthetic construct; identical to SEQ ID NO: 11
  REGION 1..144: note = Description of Artificial Sequence: Synthetic polypeptide

SEQ ID NO: 55 - protein, 143 aa, synthetic construct
  REGION 1..143: note = Description of Artificial Sequence: Synthetic polypeptide
MTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 56 - protein, 142 aa, synthetic construct
  REGION 1..142: note = Description of Artificial Sequence: Synthetic polypeptide
MTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 57 - protein, 141 aa, synthetic construct
  REGION 1..141: note = Description of Artificial Sequence: Synthetic polypeptide
MTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 58 - protein, 140 aa, synthetic construct
  REGION 1..140: note = Description of Artificial Sequence: Synthetic polypeptide
MTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 59 - protein, 143 aa, synthetic construct; identical to SEQ ID NO: 12
  REGION 1..143: note = Description of Artificial Sequence: Synthetic polypeptide

SEQ ID NO: 60 - protein, 142 aa, synthetic construct
  REGION 1..142: note = Description of Artificial Sequence: Synthetic polypeptide
MEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 61 - protein, 141 aa, synthetic construct
  REGION 1..141: note = Description of Artificial Sequence: Synthetic polypeptide
MEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 62 - protein, 140 aa, synthetic construct
  REGION 1..140: note = Description of Artificial Sequence: Synthetic polypeptide
MEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 63 - protein, 139 aa, synthetic construct
  REGION 1..139: note = Description of Artificial Sequence: Synthetic polypeptide
MEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 64 - protein, 142 aa, synthetic construct; identical to SEQ ID NO: 13
  REGION 1..142: note = Description of Artificial Sequence: Synthetic polypeptide

SEQ ID NO: 65 - protein, 141 aa, synthetic construct
  REGION 1..141: note = Description of Artificial Sequence: Synthetic polypeptide
MKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 66 - protein, 140 aa, synthetic construct
  REGION 1..140: note = Description of Artificial Sequence: Synthetic polypeptide
MKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 67 - protein, 139 aa, synthetic construct
  REGION 1..139: note = Description of Artificial Sequence: Synthetic polypeptide
MKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 68 - protein, 138 aa, synthetic construct
  REGION 1..138: note = Description of Artificial Sequence: Synthetic polypeptide
MKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 69 - protein, 144 aa, synthetic construct
  REGION 1..144: note = Description of Artificial Sequence: Synthetic polypeptide
ALTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 70 - protein, 143 aa, synthetic construct
  REGION 1..143: note = Description of Artificial Sequence: Synthetic polypeptide
ALTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 71 - protein, 142 aa, synthetic construct
  REGION 1..142: note = Description of Artificial Sequence: Synthetic polypeptide
ALTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 72 - protein, 141 aa, synthetic construct
  REGION 1..141: note = Description of Artificial Sequence: Synthetic polypeptide
ALTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 73 - protein, 144 aa, synthetic construct; identical to SEQ ID NO: 20
  REGION 1..144: note = Description of Artificial Sequence: Synthetic polypeptide

SEQ ID NO: 74 - protein, 143 aa, synthetic construct
  REGION 1..143: note = Description of Artificial Sequence: Synthetic polypeptide
LTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 75 - protein, 142 aa, synthetic construct
  REGION 1..142: note = Description of Artificial Sequence: Synthetic polypeptide
LTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 76 - protein, 141 aa, synthetic construct
  REGION 1..141: note = Description of Artificial Sequence: Synthetic polypeptide
LTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 77 - protein, 140 aa, synthetic construct
  REGION 1..140: note = Description of Artificial Sequence: Synthetic polypeptide
LTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 78 - protein, 143 aa, synthetic construct; identical to SEQ ID NO: 21
  REGION 1..143: note = Description of Artificial Sequence: Synthetic polypeptide

SEQ ID NO: 79 - protein, 142 aa, synthetic construct
  REGION 1..142: note = Description of Artificial Sequence: Synthetic polypeptide
TEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 80 - protein, 141 aa, synthetic construct
  REGION 1..141: note = Description of Artificial Sequence: Synthetic polypeptide
TEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 81 - protein, 140 aa, synthetic construct
  REGION 1..140: note = Description of Artificial Sequence: Synthetic polypeptide
TEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 82 - protein, 139 aa, synthetic construct
  REGION 1..139: note = Description of Artificial Sequence: Synthetic polypeptide
TEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 83 - protein, 142 aa, synthetic construct; identical to SEQ ID NO: 22
  REGION 1..142: note = Description of Artificial Sequence: Synthetic polypeptide

SEQ ID NO: 84 - protein, 141 aa, synthetic construct
  REGION 1..141: note = Description of Artificial Sequence: Synthetic polypeptide
EKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 85 - protein, 140 aa, synthetic construct
  REGION 1..140: note = Description of Artificial Sequence: Synthetic polypeptide
EKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 86 - protein, 139 aa, synthetic construct
  REGION 1..139: note = Description of Artificial Sequence: Synthetic polypeptide
EKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 87 - protein, 138 aa, synthetic construct
  REGION 1..138: note = Description of Artificial Sequence: Synthetic polypeptide
EKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 88 - protein, 141 aa, synthetic construct; identical to SEQ ID NO: 23
  REGION 1..141: note = Description of Artificial Sequence: Synthetic polypeptide

SEQ ID NO: 89 - protein, 140 aa, synthetic construct
  REGION 1..140: note = Description of Artificial Sequence: Synthetic polypeptide
KNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 90 - protein, 139 aa, synthetic construct
  REGION 1..139: note = Description of Artificial Sequence: Synthetic polypeptide
KLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 91 - protein, 138 aa, synthetic construct
  REGION 1..138: note = Description of Artificial Sequence: Synthetic polypeptide
KPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 92 - protein, 137 aa, synthetic construct
  REGION 1..137: note = Description of Artificial Sequence: Synthetic polypeptide
KPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 93 - protein, 144 aa, synthetic construct
  REGION 1..144: note = Description of Artificial Sequence: Synthetic polypeptide
ALTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 94 - protein, 143 aa, synthetic construct
  REGION 1..143: note = Description of Artificial Sequence: Synthetic polypeptide
ALTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 95 - skipped

SEQ ID NO: 96 - protein, 142 aa, synthetic construct
  REGION 1..142: note = Description of Artificial Sequence: Synthetic polypeptide
ALTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 97 - protein, 141 aa, synthetic construct
  REGION 1..141: note = Description of Artificial Sequence: Synthetic polypeptide
ALTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 98 - protein, 144 aa, synthetic construct; identical to SEQ ID NO: 25
  REGION 1..144: note = Description of Artificial Sequence: Synthetic polypeptide

SEQ ID NO: 99 - protein, 143 aa, synthetic construct
  REGION 1..143: note = Description of Artificial Sequence: Synthetic polypeptide
LTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 100 - protein, 142 aa, synthetic construct
  REGION 1..142: note = Description of Artificial Sequence: Synthetic polypeptide
LTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 101 - protein, 141 aa, synthetic construct
  REGION 1..141: note = Description of Artificial Sequence: Synthetic polypeptide
LTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 102 - protein, 140 aa, synthetic construct
  REGION 1..140: note = Description of Artificial Sequence: Synthetic polypeptide
LTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 103 - protein, 143 aa, synthetic construct; identical to SEQ ID NO: 26
  REGION 1..143: note = Description of Artificial Sequence: Synthetic polypeptide

SEQ ID NO: 104 - protein, 142 aa, synthetic construct
  REGION 1..142: note = Description of Artificial Sequence: Synthetic polypeptide
TEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 105 - protein, 141 aa, synthetic construct
  REGION 1..141: note = Description of Artificial Sequence: Synthetic polypeptide
TEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 106 - protein, 140 aa, synthetic construct
  REGION 1..140: note = Description of Artificial Sequence: Synthetic polypeptide
TEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 107 - protein, 139 aa, synthetic construct
  REGION 1..139: note = Description of Artificial Sequence: Synthetic polypeptide
TEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 108 - protein, 142 aa, synthetic construct; identical to SEQ ID NO: 27
  REGION 1..142: note = Description of Artificial Sequence: Synthetic polypeptide

SEQ ID NO: 109 - protein, 141 aa, synthetic construct
  REGION 1..141: note = Description of Artificial Sequence: Synthetic polypeptide
EKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 110 - protein, 140 aa, synthetic construct
  REGION 1..140: note = Description of Artificial Sequence: Synthetic polypeptide
EKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 111 - protein, 139 aa, synthetic construct
  REGION 1..139: note = Description of Artificial Sequence: Synthetic polypeptide
EKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 112 - protein, 138 aa, synthetic construct
  REGION 1..138: note = Description of Artificial Sequence: Synthetic polypeptide
EKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 113 - protein, 141 aa, synthetic construct; identical to SEQ ID NO: 28
  REGION 1..141: note = Description of Artificial Sequence: Synthetic polypeptide

SEQ ID NO: 114 - protein, 140 aa, synthetic construct
  REGION 1..140: note = Description of Artificial Sequence: Synthetic polypeptide
KNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 115 - protein, 139 aa, synthetic construct
  REGION 1..139: note = Description of Artificial Sequence: Synthetic polypeptide
KLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 116 - protein, 138 aa, synthetic construct
  REGION 1..138: note = Description of Artificial Sequence: Synthetic polypeptide
KPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 117 - protein, 137 aa, synthetic construct
  REGION 1..137: note = Description of Artificial Sequence: Synthetic polypeptide
KPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 118 - protein, 146 aa, synthetic construct; identical to SEQ ID NO: 14
  REGION 1..146: note = Description of Artificial Sequence: Synthetic polypeptide

SEQ ID NO: 119 - protein, 144 aa, synthetic construct; identical to SEQ ID NO: 46
  REGION 1..144: note = Description of Artificial Sequence: Synthetic polypeptide

SEQ ID NO: 120 - protein, 143 aa, synthetic construct
  REGION 1..143: note = Description of Artificial Sequence: Synthetic polypeptide
MALTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 121 - protein, 142 aa, synthetic construct
  REGION 1..142: note = Description of Artificial Sequence: Synthetic polypeptide
MALTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 122 - protein, 145 aa, synthetic construct; identical to SEQ ID NO: 15
  REGION 1..145: note = Description of Artificial Sequence: Synthetic polypeptide

SEQ ID NO: 123 - protein, 144 aa, synthetic construct
  REGION 1..144: note = Description of Artificial Sequence: Synthetic polypeptide
MLTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 124 - protein, 143 aa, synthetic construct
  REGION 1..143: note = Description of Artificial Sequence: Synthetic polypeptide
MLTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 125 - protein, 142 aa, synthetic construct
  REGION 1..142: note = Description of Artificial Sequence: Synthetic polypeptide
MLTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 126 - protein, 141 aa, synthetic construct
  REGION 1..141: note = Description of Artificial Sequence: Synthetic polypeptide
MLTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 127 - protein, 144 aa, synthetic construct; identical to SEQ ID NO: 16
  REGION 1..144: note = Description of Artificial Sequence: Synthetic polypeptide

SEQ ID NO: 128 - protein, 143 aa, synthetic construct
  REGION 1..143: note = Description of Artificial Sequence: Synthetic polypeptide
MTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 129 - protein, 142 aa, synthetic construct
  REGION 1..142: note = Description of Artificial Sequence: Synthetic polypeptide
MTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 130 - protein, 141 aa, synthetic construct
  REGION 1..141: note = Description of Artificial Sequence: Synthetic polypeptide
MTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 131 - protein, 140 aa, synthetic construct
  REGION 1..140: note = Description of Artificial Sequence: Synthetic polypeptide
MTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 132 - protein, 143 aa, synthetic construct; identical to SEQ ID NO: 17
  REGION 1..143: note = Description of Artificial Sequence: Synthetic polypeptide

SEQ ID NO: 133 - protein, 142 aa, synthetic construct
  REGION 1..142: note = Description of Artificial Sequence: Synthetic polypeptide
MEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 134 - protein, 141 aa, synthetic construct
  REGION 1..141: note = Description of Artificial Sequence: Synthetic polypeptide
MEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 135 - protein, 140 aa, synthetic construct
  REGION 1..140: note = Description of Artificial Sequence: Synthetic polypeptide
MEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 136 - protein, 139 aa, synthetic construct
  REGION 1..139: note = Description of Artificial Sequence: Synthetic polypeptide
MEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 137 - protein, 142 aa, synthetic construct; identical to SEQ ID NO: 18
  REGION 1..142: note = Description of Artificial Sequence: Synthetic polypeptide

SEQ ID NO: 138 - protein, 141 aa, synthetic construct
  REGION 1..141: note = Description of Artificial Sequence: Synthetic polypeptide
MKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 139 - protein, 140 aa, synthetic construct
  REGION 1..140: note = Description of Artificial Sequence: Synthetic polypeptide
MKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 140 - protein, 139 aa, synthetic construct
  REGION 1..139: note = Description of Artificial Sequence: Synthetic polypeptide
MKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 141 - protein, 138 aa, synthetic construct
  REGION 1..138: note = Description of Artificial Sequence: Synthetic polypeptide
MKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 142 - protein, 140 aa, synthetic construct
  REGION 1..140: note = Description of Artificial Sequence: Synthetic polypeptide
  MOD_RES 67: note = Any amino acid
FNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 143 - protein, 139 aa, synthetic construct
  REGION 1..139: note = Description of Artificial Sequence: Synthetic polypeptide
  MOD_RES 66: note = Any amino acid
NLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 144 - protein, 137 aa, synthetic construct
  REGION 1..137: note = Description of Artificial Sequence: Synthetic polypeptide
  MOD_RES 64: note = Any amino acid
PPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 145 - protein, 136 aa, synthetic construct
  REGION 1..136: note = Description of Artificial Sequence: Synthetic polypeptide
  MOD_RES 63: note = Any amino acid
PGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 146 - protein, 140 aa, synthetic construct
  REGION 1..140: note = Description of Artificial Sequence: Synthetic polypeptide
  MOD_RES 67: note = Any amino acid
FNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 147 - protein, 139 aa, synthetic construct
  REGION 1..139: note = Description of Artificial Sequence: Synthetic polypeptide
  MOD_RES 66: note = Any amino acid
NLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 148 - protein, 137 aa, synthetic construct
  REGION 1..137: note = Description of Artificial Sequence: Synthetic polypeptide
  MOD_RES 64: note = Any amino acid
PPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 149 - protein, 136 aa, synthetic construct
  REGION 1..136: note = Description of Artificial Sequence: Synthetic polypeptide
  MOD_RES 63: note = Any amino acid
PGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 150 - protein, 144 aa, synthetic construct
  REGION 1..144: note = Description of Artificial Sequence: Synthetic polypeptide
  MOD_RES 71: note = Any amino acid
ALTEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 151 - protein, 143 aa, synthetic construct
  REGION 1..143: note = Description of Artificial Sequence: Synthetic polypeptide
  MOD_RES 70: note = Any amino acid
ALTEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 152 - protein, 143 aa, synthetic construct
  REGION 1..143: note = Description of Artificial Sequence: Synthetic polypeptide
  MOD_RES 70: note = Any amino acid
ALTEKPPGGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 153 - protein, 141 aa, synthetic construct
  REGION 1..141: note = Description of Artificial Sequence: Synthetic polypeptide
  MOD_RES 68: note = Any amino acid
ALTEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 154 - protein, 144 aa, synthetic construct
  REGION 1..144: note = Description of Artificial Sequence: Synthetic polypeptide
  MOD_RES 71: note = Any amino acid
LTEKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 155 - protein, 143 aa, synthetic construct
  REGION 1..143: note = Description of Artificial Sequence: Synthetic polypeptide
  MOD_RES 70: note = Any amino acid
LTEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 156 - protein, 142 aa, synthetic construct
  REGION 1..142: note = Description of Artificial Sequence: Synthetic polypeptide
  MOD_RES 69: note = Any amino acid
LTEKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 157 - protein, 141 aa, synthetic construct
  REGION 1..141: note = Description of Artificial Sequence: Synthetic polypeptide
  MOD_RES 68: note = Any amino acid
LTEKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 158 - protein, 140 aa, synthetic construct
  REGION 1..140: note = Description of Artificial Sequence: Synthetic polypeptide
  MOD_RES 67: note = Any amino acid
LTEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 159 - protein, 143 aa, synthetic construct
  REGION 1..143: note = Description of Artificial Sequence: Synthetic polypeptide
  MOD_RES 70: note = Any amino acid
TEKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 160 - protein, 142 aa, synthetic construct
  REGION 1..142: note = Description of Artificial Sequence: Synthetic polypeptide
  MOD_RES 69: note = Any amino acid
TEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 161 - protein, 141 aa, synthetic construct
  REGION 1..141: note = Description of Artificial Sequence: Synthetic polypeptide
  MOD_RES 68: note = Any amino acid
TEKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 162 - protein, 140 aa, synthetic construct
  REGION 1..140: note = Description of Artificial Sequence: Synthetic polypeptide
  MOD_RES 67: note = Any amino acid
TEKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 163 - protein, 139 aa, synthetic construct
  REGION 1..139: note = Description of Artificial Sequence: Synthetic polypeptide
  MOD_RES 66: note = Any amino acid
TEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 164 - protein, 142 aa, synthetic construct
  REGION 1..142: note = Description of Artificial Sequence: Synthetic polypeptide
  MOD_RES 69: note = Any amino acid
EKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 165 - protein, 141 aa, synthetic construct
  REGION 1..141: note = Description of Artificial Sequence: Synthetic polypeptide
  MOD_RES 68: note = Any amino acid
EKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 166 - protein, 140 aa, synthetic construct
  REGION 1..140: note = Description of Artificial Sequence: Synthetic polypeptide
  MOD_RES 67: note = Any amino acid
EKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 167 - protein, 139 aa, synthetic construct
  REGION 1..139: note = Description of Artificial Sequence: Synthetic polypeptide
  MOD_RES 66: note = Any amino acid
EKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 168 - protein, 138 aa, synthetic construct
  REGION 1..138: note = Description of Artificial Sequence: Synthetic polypeptide
  MOD_RES 65: note = Any amino acid
EKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 169 - protein, 141 aa, synthetic construct
  REGION 1..141: note = Description of Artificial Sequence: Synthetic polypeptide
  MOD_RES 68: note = Any amino acid
KFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 170 - protein, 140 aa, synthetic construct
  REGION 1..140: note = Description of Artificial Sequence: Synthetic polypeptide
  MOD_RES 67: note = Any amino acid
KNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 171 - protein, 139 aa, synthetic construct
  REGION 1..139: note = Description of Artificial Sequence: Synthetic polypeptide
  MOD_RES 66: note = Any amino acid
KLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 172 - protein, 138 aa, synthetic construct
  REGION 1..138: note = Description of Artificial Sequence: Synthetic polypeptide
  MOD_RES 65: note = Any amino acid
KPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 173 - protein, 137 aa, synthetic construct
  REGION 1..137: note = Description of Artificial Sequence: Synthetic polypeptide
  MOD_RES 64: note = Any amino acid
KPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 174 - protein, 140 aa, synthetic construct; identical to SEQ ID NO: 146
  REGION 1..140: note = Description of Artificial Sequence: Synthetic polypeptide
  MOD_RES 67: note = Any amino acid

SEQ ID NO: 175 - protein, 145 aa, synthetic construct
  REGION 1..145: note = Description of Artificial Sequence: Synthetic polypeptide
  MOD_RES 72: note = Any amino acid
ALTEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 176 - protein, 144 aa, synthetic construct
  REGION 1..144: note = Description of Artificial Sequence: Synthetic polypeptide
  MOD_RES 71: note = Any amino acid
LTEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 177 - protein, 143 aa, synthetic construct
  REGION 1..143: note = Description of Artificial Sequence: Synthetic polypeptide
  MOD_RES 70: note = Any amino acid
TEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 178 - protein, 142 aa, synthetic construct
  REGION 1..142: note = Description of Artificial Sequence: Synthetic polypeptide
  MOD_RES 69: note = Any amino acid
EKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 179 - protein, 141 aa, synthetic construct
  REGION 1..141: note = Description of Artificial Sequence: Synthetic polypeptide
  MOD_RES 68: note = Any amino acid
KFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 180 - protein, 141 aa, synthetic construct; identical to SEQ ID NO: 179
  REGION 1..141: note = Description of Artificial Sequence: Synthetic polypeptide
  MOD_RES 68: note = Any amino acid

SEQ ID NO: 181 - protein, 144 aa, synthetic construct
  REGION 1..144: note = Description of Artificial Sequence: Synthetic polypeptide
  MOD_RES 71: note = Any amino acid
ALTEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 182 - protein, 143 aa, synthetic construct; identical to SEQ ID NO: 151
  REGION 1..143: note = Description of Artificial Sequence: Synthetic polypeptide
  MOD_RES 70: note = Any amino acid

SEQ ID NO: 183 - protein, 142 aa, synthetic construct
  REGION 1..142: note = Description of Artificial Sequence: Synthetic polypeptide
  MOD_RES 69: note = Any amino acid
ALTEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 184 - protein, 141 aa, synthetic construct
  REGION 1..141: note = Description of Artificial Sequence: Synthetic polypeptide
  MOD_RES 68: note = Any amino acid
ALTEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 185 - protein, 144 aa, synthetic construct; identical to SEQ ID NO: 176
  REGION 1..144: note = Description of Artificial Sequence: Synthetic polypeptide
  MOD_RES 71: note = Any amino acid

SEQ ID NO: 186 - protein, 143 aa, synthetic construct
  REGION 1..143: note = Description of Artificial Sequence: Synthetic polypeptide
  MOD_RES 70: note = Any amino acid
LTEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 187 - protein, 142 aa, synthetic construct
  REGION 1..142: note = Description of Artificial Sequence: Synthetic polypeptide
  MOD_RES 69: note = Any amino acid
LTEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 188 - protein, 141 aa, synthetic construct
  REGION 1..141: note = Description of Artificial Sequence: Synthetic polypeptide
  MOD_RES 68: note = Any amino acid
LTEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 189 - protein, 140 aa, synthetic construct
  REGION 1..140: note = Description of Artificial Sequence: Synthetic polypeptide
  MOD_RES 67: note = Any amino acid
LTEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 190 - protein, 143 aa, synthetic construct; identical to SEQ ID NO: 177
  REGION 1..143: note = Description of Artificial Sequence: Synthetic polypeptide
  MOD_RES 70: note = Any amino acid

SEQ ID NO: 191 - protein, 142 aa, synthetic construct
  REGION 1..142: note = Description of Artificial Sequence: Synthetic polypeptide
  MOD_RES 69: note = Any amino acid
TEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 192 - protein, 141 aa, synthetic construct
  REGION 1..141: note = Description of Artificial Sequence: Synthetic polypeptide
  MOD_RES 68: note = Any amino acid
TEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 193 - protein, 140 aa, synthetic construct
  REGION 1..140: note = Description of Artificial Sequence: Synthetic polypeptide
  MOD_RES 67: note = Any amino acid
TEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 194 - protein, 139 aa, synthetic construct
  REGION 1..139: note = Description of Artificial Sequence: Synthetic polypeptide
  MOD_RES 66: note = Any amino acid
TEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 195 - protein, 142 aa, synthetic construct; identical to SEQ ID NO: 178
  REGION 1..142: note = Description of Artificial Sequence: Synthetic polypeptide
  MOD_RES 69: note = Any amino acid

SEQ ID NO: 196 - protein, 141 aa, synthetic construct
  REGION 1..141: note = Description of Artificial Sequence: Synthetic polypeptide
  MOD_RES 68: note = Any amino acid
EKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 197 - protein, 140 aa, synthetic construct
  REGION 1..140: note = Description of Artificial Sequence: Synthetic polypeptide
  MOD_RES 67: note = Any amino acid
EKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 198 - protein, 139 aa, synthetic construct
  REGION 1..139: note = Description of Artificial Sequence: Synthetic polypeptide
  MOD_RES 66: note = Any amino acid
EKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 199 - protein, 138 aa, synthetic construct
  REGION 1..138: note = Description of Artificial Sequence: Synthetic polypeptide
  MOD_RES 65: note = Any amino acid
EKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 200 - protein, 141 aa, synthetic construct; identical to SEQ ID NO: 179
  REGION 1..141: note = Description of Artificial Sequence: Synthetic polypeptide
  MOD_RES 68: note = Any amino acid

SEQ ID NO: 201 - protein, 140 aa, synthetic construct
  REGION 1..140: note = Description of Artificial Sequence: Synthetic polypeptide
  MOD_RES 67: note = Any amino acid
KNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 202 - protein, 139 aa, synthetic construct
  REGION 1..139: note = Description of Artificial Sequence: Synthetic polypeptide
  MOD_RES 66: note = Any amino acid
KLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 203 - protein, 138 aa, synthetic construct
  REGION 1..138: note = Description of Artificial Sequence: Synthetic polypeptide
  MOD_RES 65: note = Any amino acid
KPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 204 - protein, 137 aa, synthetic construct
  REGION 1..137: note = Description of Artificial Sequence: Synthetic polypeptide
  MOD_RES 64: note = Any amino acid
KPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 205 - protein, 140 aa, synthetic construct
  REGION 1..140: note = Description of Artificial Sequence: Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 206 - protein, 140 aa, synthetic construct
  REGION 1..140: note = Description of Artificial Sequence: Synthetic polypeptide
FNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 207 - protein, 145 aa, synthetic construct
  REGION 1..145: note = Description of Artificial Sequence: Synthetic polypeptide
MALTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD